SEQUENCE LISTING (25 sequences)

SEQ ID NO: 1 - DNA, 8196 bp, Homo sapiens
agtggcgctgctgggacgggggaaaggagacgcttcttcctcttgctgctcttctcgttcccgagatcagcggcggcggtgaccgcgagtgggtcggcaccgtctccggctccgggtgcgaacaatgctgactgatagcggaggcggcggcacctcctttgaggaggacctggactctgtggctccgcgatccgccccagctggggcctcggagccgcctccgccgggaggggtcggtctggggatccgcaccgtgaggctctttggggaggccgggccagcgtcgggagtcggcagcagcggcggcggcggcagcggcagcggtacgggcggaggggacgcggcgctggatttcaagttggcggctgccgtgctgaggaccgggggtggaggtggtgcctctggcagtgacgaggacgaagtgtccgaggttgaatcatttattttggaccaagaagatctggataacccagtgcttaaaacaacatcagagatattcttatcaagtactgcagaaggagcagacttacgcactgtggatccagagacacaggcacgactagaagcattgctagaagcagcaggaattggcaaattgtcaactgctgatggtaaagcttttgcagatcctgaggtactccggagactgacatcctcagttagttgtgcactggatgaagctgctgctgcactgacacggatgaaagcagaaaacagccacaatgcaggacaagtggacactcgcagtctagcagaagcttgttcagatggggatgttaatgctgttcgtaaattgctagatgaaggcagaagtgtaaatgaacatacagaagaaggagaaagcctgctgtgtttggcttgttcagcagggtattatgaattagcacaagtattgcttgctatgcatgctaatgttgaagatcgagggaataaaggagacataactcccctgatggcagcttccagtggaggttacttagatattgtgaaattattacttcttcatgatgctgatgtcaactcccagtctgcaacaggaaacactgcgctaacttatgcatgtgctggaggatttgttgacattgttaaagtgctccttaatgaaggtgcaaatatagaagatcataatgaaaatggacatactcccttaatggaagcagccagtgcaggtcatgtggaagttgcaagagttcttttagatcatggtgcaggcatcaacactcattctaatgaattcaaagaaagtgctctaacacttgcttgctacaaaggccatttggatatggttcgctttctacttgaagctggtgcagatcaagagcacaaaacagatgagatgcacactgccttaatggaggcctgcatggatggacatgtagaggtggcacgtttgcttttggatagtggtgctcaagtgaacatgcctgcagattcatttgaatctccattgacgctagctgcctgtggaggacatgttgaattggcagctctacttattgaaaggggagcaaatcttgaagaagttaatgatgaaggatacactcccttgatggaagctgcccgggaaggacatgaagaaatggtggcactactcttagcacaaggagcaaatataaatgcccagacagaagaaactcaagaaactgctcttactttggcttgctgtggaggattttctgaagttgcagactttcttattaaggcaggggctgatatagaacttggctgctccacacctctgatggaggcatctcaggagggacacctggaattggttaaatatttgctggcttctggcgctaatgtgcatgctacaacagcaacaggagacacagccttaacctatgcttgtgaaaatggacatacggatgttgcagatgttttacttcaagcaggggctgatttagaacatgaatctgaaggtggaagaacacctttgatgaaagctgcaagagctggtcatttgtgcactgtgcagtttcttattagcaaaggtgccaatgttaacagggctacagccaataatgatcatacagtagtgtcgctggcatgtgcaggaggccacctggcagttgttgagcttctcttggctcatggggctgaccctactcatcgactcaaggatggttcaacaatgctcattgaagctgcaaagggtggccatactaatgtagtttcttatctgttggattatccaaataatgttctgtcagttcccaccacagatgtgtctcagctccctccaccttctcaagatcagtctcaggtgccacgtgtgccaacgcatacacttgccatggttgtacctccccaggaacctgacagaacttcacaggagaactctcctgcccttttaggagtgcaaaaaggtacatccaagcagaagtccagttccctccaggtagcagatcaggacctactgccatcttttcacccataccagcctttggagtgcatagtagaggagactgaaggcaagctgaatgaactgggacaaagaattagtgctattgaaaaagcacagcttaagtcactggagttaattcaaggtgaacctctgaacaaagataagatagaagaacttaaaaagaacagagaagagcaagtccagaagaagaagaaaatattgaaagaactgcagaaagtggaaaggcagttgcagatgaaaacacagcagcaatttaccaaagaatacttggaaaccaaaggtcagaaagacacagtgtctctacaccaacagtgctctcatagaggagtcttcccagaaggggaaggagatggtagtctcccagaggatcacttttcagagttacctcaggttgacacaatcttatttaaagataatgatgttgatgatgagcaacagtctccaccatcggcagaacagattgattttgtcccagtccagcctttatcatctccacagtgtaacttttccagtgacttaggttctaatgggacaaattctcttgaacttcagaaagtatcaggtaatcagcagattgtaggacagcctcagattgctattactggacatgatcaggggctgttagttcaagaaccagatggactaatggttgcaactccagctcagacgcttaccgacactcttgatgacctgatagcagctgtgagtaccagagtgcccactggttccaacagttcttctcagaccacagagtgtcttacacctgaatcctgttcgcagactacaagcaatgtggcttcccaatcgatgcctcctgtgtatccttcagttgacattgatgcacatactgagagcaatcatgacacagcattaacactagcttgtgcaggtggtcatgaagaacttgtatctgtgctcattgcacgggatgccaaaattgaacacagagacaaaaaaggtttcacaccactaatcctggcagcaacagcagggcatgttggagttgttgaaatccttttggataaaggtggagatatagaagcacagtctgaacgaactaaggatactccgctttcattggcatgttctggtggacgtcaggaggtggtagacttgctgctggctcgaggtgcaaataaagaacataggaacgtatctgattatacaccactgagtctagctgcgtctggaggatatgttaatatcattaagattctgcttaatgctggggcagaaattaattcaaggactgggagtaaactaggtatttctcccctgatgttggctgcaatgaatggacatgttcctgcagtaaaattgctgctcgatatgggttcagacattaatgcccaaatagagaccaatcggaacacggctctcaccctggcctgtttccagggccgagcagaagtagtgagtttgcttctggaccgaaaagccaatgttgaacatagggcaaagacgggtcttacccccttgatggaagcagcttctggagggtatgcagaggttggaagagttcttcttgataaaggagcagatgttaatgctccccctgtgccttcctcaagagatactgctttaacaatagcagcagacaaaggtcactacaaattttgtgaactcctgattcataggggagcccacattgatgttcgtaacaaaaagggaaatacgccactttggctggcatccaatggaggtcattttgatgttgtgcagttgctagtgcaagcaggtgctgatgtggatgcagcagataaccggaaaatcacacctcttatgtcagcatttcgcaagggtcatgtaaaagttgttcaatatttggtaaaggaagtaaatcagttcccttctgatatagaatgcatgagatacatagcaacaattacagataaggaactgttgaaaaaatgtcatcaatgtgtcgaaaccattgtgaaggctaaagaccagcaagctgcagaagcaaataagaatgcgagtattcttttaaaggaacttgatctggaaaagtcaagagaagagagcagaaagcaggctcttgctgctaaaagagaaaaaagaaaagaaaagagaaaaaagaaaaaagaggaacagaaaaggaaacaggaagaagatgaagaaaacaaacctaaggagaattcggaactaccagaggatgaagatgaagaggagaatgatgaagatgtggagcaagaagttcccatagaacctcctagtgcaaccaccaccactacgattggaatctctgcaacatctgcaacattcacaaatgtgtttgggaaaaaaagggccaatgtggtgacaactcccagcaccaatcggaaaaataagaagaacaaaacaaaagaaacccctcctacagcacatttaattttaccagaacaacatatgtctttagcccaacaaaaggcagataaaaataaaataaatggagaacctagaggtggtggtgcaggtgggaatagtgattcagataacttggacagcacagactgcaacagtgagagtagcagtggtggtaaaagccaagagttaaattttgtgatggatgtgaattcctctaaatacccctcactgctccttcattcccaagaagaaaagacaagtactgctacttccaaaactcagacacgacttgaaggtgaagtgactcctaattccttgtcaaccagctacaagacagtgtcattgccattaagctctccaaacataaagctgaatctcactagccctaaaaggggtcagaaaagagaagaagggtggaaagaagttgtacgaaggtcaaagaaattgtctgttccagcctcagtggtgtcgaggataatgggaagaggaggatgcaacatcactgcaatacaggatgttactggtgcccatattgatgtggataaacaaaaagataagaatggcgagagaatgatcacaataaggggtggcacagaatcaacaagatatgcagttcaactaatcaatgcactcattcaagatcctgctaaggaactggaagacttgattcctaaaaatcatatcagaacacctgccagcaccaaatcaattcatgctaacttctcatctggagtaggtaccacagcagcttccagtaaaaatgcatttcctttgggtgctccaactcttgtaacttcacaggcaacaacgttatctacgttccagcccgctaataaacttaataagaatgttccaacaaatgtacgttcttctttcccagtttctctacccttagcttatcctcaccctcattttgccctgctggctgctcaaactatgcaacagattcggcatcctcgcttacccatggcccagtttggaggaaccttctcaccttctcctaacacatggggaccattcccagtgagacctgtgaatcctggcaacacaaatagctctccaaagcataataacacaagccgtctacctaaccagaacgggactgttttaccctcagagtctgctggactagctactgccagttgtcctatcactgtctcttctgtagttgctgccagtcagcaactgtgtgtcactaatacccggactccttcatcagtcagaaagcagttgtttgcctgtgtgcctaagacaagtcctccagcaacagtgatttcttctgtgacaagcacttgtagttccctgccttctgtctcctctgcacctatcactagcgggcaagctcccaccacatttctacctgcaagtacttctcaagcacagctttcttcacaaaagatggagtctttctctgctgtgccacccaccaaagagaaagtgtccacacaggaccagcccatggcaaacctatgtaccccatcttcaactgcaaacagttgcagtagctctgccagcaacaccccgggagctccagaaactcacccatccagtagtcccactcctacttccagtaacacacaagaggaggcacagccatccagtgtgtctgatttaagtcctatgtcaatgccttttgcatctaactcagaacctgctccattgactttgacatcacccagaatggttgctgctgataatcaggacaccagtaatttacctcagttagctgtaccagcacctcgagtttctcatcgaatgcagcccagaggttctttttactccatggtaccaaatgcaactattcaccaggatccccagtctatttttgttacgaatccagttactttaacaccacctcaaggcccaccagctgcagtgcagctttcttcagctgtgaacattatgaatggttctcagatgcacataaacccagcaaataagtctttgccacctacatttggcccagccacacttttcaatcacttcagcagtctttttgatagtagtcaggtgccagctaaccagggctggggagatggtccactgtcctcacgagttgctacagatgcctctttcactgttcagtcagcgttcctgggtaactcagtgcttggacacttggaaaacatgcaccctgataactcaaaggcacctggcttcagaccaccttcccagcgagtttctactagtccagttgggttaccatccattgacccatcaggcagctccccatcttcctcttctgctcctctggcaagtttttccggcataccaggaacaagggttttcctgcaagggccagctcctgttgggactcctagtttcaacagacaacatttttctccccatccttggacaagcgcctcaaactcatccacttctgccccaccaacgttgggccaaccaaaaggagtcagtgccagtcaagatcgaaagatacctcccccaattggaacagagagactggcccgaattcggcaaggagggtctgttgcacaagccccggcggggaccagttttgtcgctcccgttggacacagtggaatctggtcatttggtgtcaatgctgtgtcagaaggcttatcaggttggtcgcaatctgtgatggggaaccatccaatgcatcaacaattatcagacccaagcacattctcccaacatcagccaatggagagagatgattctggaatggtagccccctctaacatttttcatcagcctatggcaagtggttttgtggatttttctaaaggtctgccaatttccatgtatggaggcaccataataccctctcatcctcagcttgctgatgttccaggaggccctctgtttaatggacttcacaatccagatcctgcttggaaccctatgataaaagttatccaaaattcaactgaatgcactgatgcccagcagatttggcctggcacgtgggcacctcatattggaaacatgcatctcaaatatgtcaactaagttagaaggtctttactctttagccttgtttaagaaacctatgaccttggaagaaccatggggatttttttttaatgtgcctaagaaattttctctgaggctttagcaatggaaatttgattgcccattgtataagaacaaattgatttcctatccacctgattatgttctctggttagtttagccattttgaacttaagatcatatgaccttagtgcttttggctaaacatacagaatactacttgtatgcagaagagaattagttgattacatgtttcaaccttttagggtgataaatacatgtataattgtttacatacttaaaaggaaaaagttgagtaaatttcttgtcatatagtggctctacgtaatgtagcctgtattaatgtgaaatatttaccagaatattcaataaaaagatgaacagtctttagaaaaaaa

SEQ ID NO: 2 - DNA, 1320 bp, Homo sapiens
cggaaggggaagggggtggaggttgctgctatgagagagaaaaaaaaaacagccacaatagagattctgccttcaaaggttggcttgccacctgaagcagccactgcccagggggtgcaaagaagagacagcagcgcccagcttggaggtgctaactccagaggccagcatcagcaactgggcacagaaaggagccgcctgggcagggaccatggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcatccttgtgatcttctctggaatgttccttgttttcaccctggccggggccctgttcctccatcaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccctgagccagcacctgcgggagctgcactacagccctggcctccacccccaccccgccgaccatccaagggagagtgagacctggcagccacaactgcagtcccatcctcttgtcagggccctttcctgtgtacacgtgacagagtgccttttcgagactggcagggacgaggacaaatatggatgaggtggagagtgggaagcaggagcccagccagctgcgcctgcgctgcaggagggcgggggctctggttgtaaaacacacttcctgctgcgaaagacccacatgctacaagacgggcaaaataaagtgacagatgaccaccctgca

SEQ ID NO: 3 - DNA, 3353 bp, Homo sapiens
aggagctatgaatattaatgaaagtggtcctgatgcatgcatattaaacatgcatcttacatatgacacatgttcaccttggggtggagacttaatatttaaatattgcaatcaggccctatacatcaaaaggtctattcaggacatgaaggcactcaagtatgcaatctctgtaaacccgctagaaccagtcatggtcggtgggctccttaccaggagaaaattaccgaaatcactcttgtccaatcaaagctgtagttatggctggtggagttcagttagtcagcatctggtggagctgcaagtgttttagtattgtttatttagaggccagtgcttatttagctgctagagaaaaggaaaacttgtggcagttagaacatagtttattcttttaagtgtagggctgcatgacttaacccttgtttggcatggccttaggtcctgtttgtaatttggtatcttgttgccacaaagagtgtgtttggtcagtcttatgacctctattttgacattaatgctggttggttgtgtctaaaccataaaagggaggggagtataatgaggtgtgtctgacctcttgtcctgtcatggctgggaactcagtttctaaggtttttctggggtcctctttgccaagagcgtttctattcagttggtggaggggacttaggattttatttttagtttgcagccagggtcagtacatttcagtcacccccgcccagccctcctgatcctcctgtcattcctcacatcctgtcattgtcagagattttacagatatagagctgaatcatttcctgccatctcttttaacacacaggcctcccagatctttctaacccaggacctacttggaaaggcatgctgggtctcttccacagactttaagctctccctacaccagaatttaggtgagtgctttgaggacatgaagctattcctcccaccaccagtagccttgggctggcccacgccaactgtggagctggagcgggagggaggagtacagacatggaattttaattctgtaatccagggcttcagttatgtacaacatccatgccatttgatgattccaccactccttttccatctcccagaagcctgctttttaatgcccgcttaatattatcagagccgagcctggaatcaaactgcctctttcaaaacctgccactatatcctggctttgtgacctcagccaagttgcttgactattctcagtctcagtttctgcacctgtcaaatagggtttatgttaacctaactttcagggctgtcaggattaaatgagcatgaaccacataaaatgtttggtgtatagtaagtgtacagtaaatacttccattatcagtccctgcaattctatttttcttccttctctacacagcccctgtctggctttaaaatgtcctgccctgctttttatgagtggatacccccagccctatgtggattagcaagttaagtaatgacactcagagacagttccatctttgtccataacttgctctgtgatccagtgtgcatcactcaaacagactatctcttttctcctacaaaacagacagctgcctctcagataatgttgggggcataggaggaatgggaagcccgctaagagaacagaagtcaaaaacagttgggttctagatgggaggaggtgtgcgtgcacatgtatgtttgtgtttcaggtcttggaatctcagcaggtcagtcacattgcagtgtgtcgcttcacctggctccctcttttaaagattttccttccctctttccaactccctgggtcctggatcctccaacagtgtcagggttagatgccttttatgggccacttgcattagtgtcctgatagaggcttaatcactgctcagaaactgccttctgcccactggcaaagggaggcaggggaaatacatgattctaattaatggtccaggcagagaggacactcagaatttcaggactgaagagtatacatgtgtgtgatggtaaatgggcaaaaatcatcccttggcttctcatgcataatgcatgggcacacagactcaaaccctctctcacacacatacacatatacattgttattccacacacaaggcataatcccagtgtccagtgcacatgcatacacgcacacattcccttcctaggccactgtattgctttcctagggcatcttcttataagacaccagtcgtataaggagcccaccccactcatctgagcttatcaaccaattacattaggaaagactgtatttcctagtaaggtcacattcagtagtactgagggttgggacttcaacacagctttttgggggatcataattcaacccatgacagccactgagattattatatctccagagaataaatgtgtggagttaaaaggaagatacatgtggtacaaggggtggtaaggcaagggtaaaaggggagggaggggattgaactagacacagacacatgagcaggactttggggagtgtgttttatatctgtcagatgcctagaacagcacctgaaatatgggactcaatcattttagtccccttctttctataagtgtgtgtgtgcggatatgtgtgctagatgttcttgctgtgttaggaggtgataaacatttgtccatgttatataggtggaaagggtcagactactaaattgtgaagacatcatctgtctgcatttattgagaatgtgaatatgaaacaagctgcaagtattctataaatgttcactgttattagatattgtatgtctttgtgtccttttattcatgaattcttgcacattatgaagaaagagtccatgtggtcagtgtcttacccggtgtagggtaaatgcacctgatagcaataacttaagcacacctttataatgaccctatatggcagatgctcctgaatgtgtgtttcgagctagaaaatccgggagtggccaatcggagattcgtttcttatctataatagacatctgagcccctggcccatcccatgaaacccaggctgtagagaggattgaggccttaagttttgggttaaatgacagttgccaggtgtcgctcattagggaaaggggttaagtgaaaatgctgtataaactgcatgatgtttgcaggcagttgtggttttcctgcccagcctgccaccaccgggccatgcggatatgttgtccagcccaacaccacaggaccatttctgtatgtaagacaattctatccagcccgccacctctggactccctcccctgtatgtaagccctcaataaaaccccacgtctcttttgctggcaaaaaaaaaaaaaaaaaaaaaaaaaaaaaaaaaaaaaaaaaaaaaaaaa

SEQ ID NO: 4 - DNA, 7479 bp, Homo sapiens
tcctcagaggcaggaaatactttccaggcaatctgggcctggttgtggaggccccttttgcaaaacctcagtctgaatttagtagacagaagtcactaggaatgccttgacaggatcctgccttagctaaggctccctccagctgcagagggtgtttttgttagactcacacactgcgtgaaactgctcagaatagagccatgatctcaaccacgaaatgggaacttagattttggagaaactaacggggacggacttctttcctagcctgagtgttgagcagtgtcatgccttggcgtttcagctcctcgttgtctaggtggtgaaatgacagaactcattcgcttctttgattggtgattttgaaataatctttcatcaagttccatctcctttaccctcatatggaatatatctctctgtctgttgttaaactacgatgacatgtctgtagctatcagaaagagaagctgggaagaacatgtgacccactggatgggacagccttttaattctgatgatcgtaacacagcatgtcatcatggactagtagctgacagcttgcaggcaagtatggaaaaagatgcaactctaaatgtggaccgcaaagagaagtgtgtttcactacctgactgctgtcatggatcagagctgagagattttcctgggaggccaatgggtcatctttcaaaggatgtggacgaaaatgacagccatgaaggtgaagatcagtttctttctctggaagccagcacagaaacactagtgcatgtttctgatgaggataacaatgctgatttatgccttacagatgataaacaggttttaaatacccaagggcagaaaacatcaggccaacatatgatccaaggagcaggctccttagaaaaggcactgcccatcatacaaagtaaccaagtttcttctaactcctggggaatagctggtgaaactgaattagcactggtaaaagaaagtggggagagaaaagttactgactctataagtaaaagcctggagctttgcaatgaaataagcttaagtgaaataaaagatgcacccaaagtaaatgcagtggatactttgaacgtgaaagatattgcacctgagaaacaattgcttaactctgctgtaattgctcagcaacgaaggaaacctgacccccctaaagatgaaaatgaaagaagcacctgcaatgtagtacaaaatgagttcttggatactccttgcacaaacagaggactgccattattaaaaacagattttggaagctgccttctgcagcctccttcctgccccaatggaatgtcagctgaaaatggcctggagaagagtggtttttcacaacatcaaaacaaaagtccaccaaaggtcaaggcagaagatggcatgcagtgtttacaattaaaggagaccctggccacccaggaacccacagataaccaagtcagacttcgtaagagaaaggaaataagagaagatcgagatagggcgcggctggactccatggtgctgctgattatgaaactggaccagcttgatcaggacatagaaaatgccctcagcaccagctcctctccatcaggcacaccaacaaacctgcggcggcacgttcctgatctggaatcaggatctgaaagtggagcagataccatttcagtaaatcagacacgagtaaatttgtcttctgacactgagtccacggacctcccatcttccactccagtagccaattctggaaccaaacccaagactacggctattcaaggtatttcagagaaggaaaaggctgaaattgaagccaaggaagcttgtgattggctacgggcaactggtttcccccagtatgcacagctttatgaagatttcctgttccccatcgatatttccttggtcaagagagagcatgattttttggacagagatgccattgaggctctatgcaggcgtctaaatactttaaacaaatgtgcggtgatgaagctagaaattagtcctcatcggaaacgaagtgacgattcagacgaggatgagccttgtgccatcagtggcaaatggactttccaaagggacagcaagaggtggtcccggcttgaagagtttgatgtcttttctccaaaacaagacctggtccctgggtccccagacgactcccacccgaaggacggccccagccccggaggcacgctgatggacctcagcgagcgccaggaggtgtcttccgtccgcagcctcagcagcactggcagcctccccagccacgcgccccccagcgaggatgctgccaccccccggactaactccgtcatcagcgtttgctcctccagcaacttggcaggcaatgacgactctttcggcagcctgccctctcccaaggaactgtccagcttcagcttcagcatgaaaggccacgaaaaaactgccaagtccaagacgcgcagtctgctgaaacggatggagagcctgaagctcaagagctcccatcacagcaagcacaaagcgccctcaaagctggggttgatcatcagcgggcccatcttgcaagaggggatggatgaggagaagctgaagcagctcaactgcgtggagatctccgccctcaatggcaaccgcatcaacgtccccatggtacgaaagaggagcgtttccaactccacgcagaccagcagcagcagcagccagtcggagaccagcagcgcggtcagcacgcccagccctgttacgaggacccggagcctcagtgcgtgcaacaagcgggtgggcatgtacttagagggcttcgatcctttcaatcagtcaacatttaacaacgtggtggagcagaactttaagaaccgcgagagctacccagaggacacggtgttctacatccctgaagatcacaagcctggcactttccccaaagctctcaccaatggcagtttctccccctcggggaataacggctctgtgaactggaggacgggaagcttccacggccctggccacatcagcctcaggagggaaaacagtagcgacagccccaaggaactgaagagacgcaattcttccagctccatgagcagccgcctgagcatctacgacaacgtgccgggctccatcctctactccagttcaggggacctggcggatctggagaacgaggacatcttccccgagctggacgacatcctctaccacgtgaaggggatgcagcggatagtcaatcagtggtcggagaagttttctgatgagggagattcggactcagccctggactcggtctctccctgcccgtcctctccaaaacagatacacctggatgtggacaacgaccgaaccacacccagcgacctggacagcacaggcaactccctgaatgaaccggaagagccctccgagatcccggaaagaagggattctggggttggggcttccctaaccaggtccaacaggcaccgactgagatggcacagtttccagagctcacatcggccaagcctcaactctgtatcactacagattaactgccagtctgtggcccagatgaacctgctgcagaaatactcactcctaaagctaacggccctgctggagaaatacacaccttctaacaagcatggttttagctgggccgtgcccaagttcatgaagaggatcaaggttccagactacaaggaccggagtgtgtttggggtcccactgacggtcaacgtgcagcgcacaggacaaccgttgcctcagagcatccagcaggccatgcgatacctccggaaccattgtttggatcaggttgggctcttcagaaaatcgggggtcaagtcccggattcaggctctgcgccagatgaatgaaggtgccatagactgtgtcaactacgaaggacagtctgcttatgacgtggcagacatgctgaagcagtattttcgagatcttcctgagccactaatgacgaacaaactctcggaaacctttctacagatctaccaatatgtgcccaaggaccagcgcctgcaggccatcaaggctgccatcatgctgctgcctgacgagaaccgggaggttctgcagaccctgctttatttcctgagcgatgtcacagcagccgtaaaagaaaaccagatgaccccaaccaacctggccgtgtgcttagcgccttccctcttccatctcaacaccctgaagagagagaattcctctcccagggtaatgcaaagaaaacaaagtttgggcaaaccagatcagaaagatttgaatgaaaacctagctgccactcaagggctggcccatatgatcgccgagtgcaagaagcttttccaggttcccgaggaaatgagccgatgtcgtaattcctataccgaacaagagctgaagcccctcactctggaagcactcgggcacctgggtaatgatgactcagctgactaccaacacttcctccaggactgtgtggatggcctgtttaaagaagtcaaagagaagtttaaaggctgggtcagctactccacttcggagcaggctgagctgtcctataagaaggtgagcgaaggaccccctctgaggctttggaggtcagtcattgaagtccctgctgtgccagaggaaatcttaaagcgcctacttaaagaacagcacctctgggatgtagacctgttggattcaaaagtgatcgaaattctggacagccaaactgaaatttaccagtatgtccaaaacagtatggcacctcatcctgctcgagactacgttgttttaagaacctggaggactaatttacccaaaggagcctgtgcccttttactaacctctgtggatcacgatcgcgcacctgtggtgggtgtgagggttaatgtgctcttgtccaggtatttgattgaaccctgtgggccaggaaaatccaaactcacctacatgtgcagagttgacttaaggggccacatgccagaatggtacacaaaatcttttggacatttgtgtgcagctgaagttgtaaagatccgggattccttcagtaaccagaacactgaaaccaaagacaccaaatctaggtgatcactgaagcaacgcaaccgcttccaccaccatggtgtttgtttctagaacttttgccagtccttgaagaatgggttctgtgtctaatcctgaaacaaagaaaactacaagctggagtgtaggaattgactatagcaatttgatacatttttaaagctgcttcctgtttgttgagggtctgtattcatagaccttgactggaatatgtaagactgtgcaaaaaaaaaaaaaaaatttatgtgtattcttattcaaattgcttctgagaagcaaaactctttaaatacattatggaagataatgaagatacttcattctcttgtgatatcagtgtatgcgtacctgtgtcgctttatttgcagtgtgttgagggactggtgtatccactggaatagttggtactcttggatgtgttttctcaccaagatgagcaaagaaaggtttgcacagaggagtgtgaatgtgtgtttgttgctggctgaatggcaatagatgtctaaggtggattcagtgtctggcacactgagacacctccaagaaggagattgatgcatcaggttcagtttaacctggaatatctgactacccctgaatccacccagaaagggggcccaacacccttgtccatttatgggtattttttttcgaagttattaagcatattccttttccacgaacctcttctgtactttgattgtaataggttggctcttacacccattccaaatgcagtttatttttagacccgattgcaaatagtgatgtagttttaaccagtatggattagttcagggatgaactgctccctctagccttactggctctgatccacagggttttgttttgttttgttttgttttttgtttaagtcgagatataaaaactgaacacgataacacttactcttaaatcaagcatcaacactttttccctgttagaattctttgcatttttgtgtttgtaacagaaacgccttaagacactatgtttgggaatataggaaactatgtgtgtcccaaggaaatccctgtaaatttaactcacctacaaaaggctttttccccgcctttggttgttaacggcattcctgaaagccacatgtgtttattcattgggcttgttcttatcagcaaataggttttctggttttatgactttttgtcttattttatttttcctacatttctttttttttttttttcctttagaatgccctggaaatatatttaagtggtaatgaaaaatagtaatcatagtaaaacgcaacaagaagaaaaccaacccaaaccagtgaagttttttagaacctttagaagggtggtctttattcaggttttactgtaatggtaaggattgactcaagagacagtattagtaaatttattgtgtatggatcaaaagtgaataatgtatgaatgagagctgtaagaaggatttttattttgttataatttagttaccattttcagtgttatttcaaaggttctttgaagaattttggggcagggcatcagattagagttttaaaatttgagtattttggatatcagtgttcctcatgaagatatacatggatattcaattttgatggcttccagatttgtaagattgtatgttgtatataccattctattaagaaacatgtccactgtgctttcaaacatagataaagcatgataaagattattatttaagatatacttgtatttatacctcagatattcttttgggttttgtacctcaaggcttttttcttcttattgtaaatacactttacgtgaatacagtctaagtgaagaaaataaataaaaggaagaggtttataacttgctctatatctgtacagattataatcaataagtgcactattattaaatgtttaaagtaagggaaaagtctgggctgccttccttaatattgcatctcactcccacccttaaaaccacagattgcaaagcatagcattttagcatcaactacaatcaaaagagcgatttgctgaaggaaaaatcggactgcaaatcattccaaggccaaactgcaactgagccacccactcccaaacaggaaaccctggtgaaggttcaggaagcacggagattctctccaacaaaggtccagttaggaaacgacgctgagaggatgacgacaacgtgcaacagcagaaagatgcttgcaagcagagtcagggtcaccagtgaatgccacaaaagttctctttcccactgtttaatttgacaagagaagaatttgaaggatatgaacattttcaagaactctgctgaggtcacttagagcgccatcacaacttatttgtgtgactaattgcctagattgtaagctctttgagggcagggcttgtctcttacacatctttataatcccctgcagcggctttcagtattttgtacttgtaggcacctaataaatttattatttgctatactg

SEQ ID NO: 5 - DNA, 2062 bp, Homo sapiens
cgggcaggcaggcggggaggacaggctgggggcggcgaccgcgaggggccgcgcgcggagggcgcctggtgcagcatgggcggcccgcgggcttgggcgctgctctgcctcgggctcctgctcccgggaggcggcgctgcgtggagcatcggggcagctccgttctccggacgcaggaactggtgctcctatgtggtgacccgcaccatctcatgccatgtgcagaatggcacctaccttcagcgagtgctgcagaactgcccctggcccatgagctgtccggggagcagctacagaactgtggtgagacccacatacaaggtgatgtacaagatagtgaccgcccgtgagtggaggtgctgccctgggcactcaggagtgagctgcgaggaagttgcagcttcctctgcctccttggagcccatgtggtcgggcagtaccatgcggcggatggcgcttcggcccacagccttctcaggttgtctcaactgcagcaaagtgtcagagctgacagagcggctgaaggtgctggaggccaagatgaccatgctgactgtcatagagcagccagtacctccaacaccagctacccctgaggaccctgccccgctctggggtccccctcctgcccagggcagccccggagatggaggcctccaggaccaagtcggtgcttgggggcttcccgggcccaccggccccaagggagatgccggcagtcggggcccaatggggatgagaggcccaccaggtccacagggccccccagggagccctggccgggctggagctgtgggcacccctggagagaggggacctcctgggccaccagggcctcctggcccccctgggcccccagcccctgttgggccaccccatgcccggatctcccagcatggagacccattgctgtccaacaccttcactgagaccaacaaccactggccccagggacccactgggcctccaggccctccagggcccatgggtccccctgggcctcctggccccacaggtgtccctgggagtcctggtcacataggacccccaggccccactggacccaaaggaatctctggccacccaggagagaagggcgagagaggactgcgtggggagcctggcccccaaggctctgctgggcagcggggggaacctggccctaagggagaccctggtgagaagagccactggggggaggggttgcaccagctacgcgaggctttgaagattttagctgagagggttttaatcttggaaacaatgattgggctctatgaaccagagctggggtctggggcgggccctgccggcacaggcacccccagcctccttcggggcaagaggggcggacatgcaaccaactaccggatcgtggcccccaggagccgggacgagagaggctgagggtggtggcggcccctgaggcagaccaggccaggcttcccctcctacctggactcggccagctgcctccagggaccgcccgtccatatttattaatgtcctcagggtcccttctgccatctaggccttaggggtaagcaggtctcagtcctggcaccatgcacatgtctgaggctgagcaagggctgagaggagaggcttgggcctcagtttccctctgtgaagtggggggaggcaggccttcaaggagggatagaggtacaaggcttcgtctcatctgctgtctgagcatccaggcccaaaggcactgagggagtcaggagctggggctcggcacatgcagagatgacagggcagggggcagtcttcctccccctccccgaccaaacctcggggagccctcctgtgcccctccctccttgttgtccagtgctgggctccccaccccgaggtcaggctgcccaatcctctgactggatcaccgggggcttcttgcctcagttcttccctctgagcccccaggccctcccgcatctcaggttggggatggggacatggagaggaaggggccgcctactcctgcaaatgcttgtgacagatgccaggaggtagatgtgtgctggccaataaaggcccctacctgattccccgcaaa

SEQ ID NO: 6 - DNA, 2431 bp, Homo sapiens
gggtggagagctgaggccgagcgaaggaaatgcaccaatcagctgctcccccgggctcacaactgtctgctgcgcccgaaaaacaagtcggtgcgctggggacccggggccggggccgccttactccggcctagccccgcggccctcggtgcgggctccagggcatgctcgggaccccccgcggctccagcccagacgccccggcctcaggtctcggcccccgcttggggccccggccgtgcggccggagggagcggccggatggagcggaggatgaaagccggatacttggaccagcaagtgccctacaccttcagcagcaaatcgcccggaaatgggagcttgcgcgaagcgctgatcggcccgctggggaagctcatggacccgggctccctgccgcccctcgactctgaagatctcttccaggatctaagtcacttccaggagacgtggctcgctgaagctcaggtaccagacagtgatgagcagtttgttcctgatttccattcagaaaacctagctttccacagccccaccaccaggatcaagaaggagccccagagtccccgcacagacccggccctgtcctgcagcaggaagccgccactcccctaccaccatggcgagcagtgcctttactccagtgcctatgacccccccagacaaatcgccatcaagtcccctgcccctggtgcccttggacagtcgcccctacagccctttccccgggcagagcaacggaatttcctgagatcctctggcacctcccagccccaccctggccatgggtacctcggggaacatagctccgtcttccagcagcccctggacatttgccactccttcacatctcagggagggggccgggaacccctcccagccccctaccaacaccagctgtcggagccctgcccaccctatccccagcagagctttaagcaagaataccatgatcccctgtatgaacaggcgggccagccagccgtggaccagggtggggtcaatgggcacaggtacccaggggcgggggtggtgatcaaacaggaacagacggacttcgcctacgactcagatgtcaccgggtgcgcatcaatgtacctccacacagagggcttctctgggccctctccaggtgacggggccatgggctatggctatgagaaacctctgcgaccattcccagatgatgtctgcgttgtccctgagaaatttgaaggagacatcaagcaggaaggggtcggtgcatttcgagaggggccgccctaccagcgccggggtgccctgcagctgtggcaatttctggtggccttgctggatgacccaacaaatgcccatttcattgcctggacgggccggggaatggagttcaagctcattgagcctgaggaggtcgccaggctctggggcatccagaagaaccggccagccatgaattacgacaagctgagccgctcgctccgatactattatgagaaaggcatcatgcagaaggtggctggtgagcgttacgtgtacaagtttgtgtgtgagcccgaggccctcttctctttggccttcccggacaatcagcgtccagctctcaaggctgagtttgaccggcctgtcagtgaggaggacacagtccctttgtcccacttggatgagagccccgcctacctcccagagctggctggccccgcccagccatttggccccaagggtggctactcttactagcccccagcggctgttccccctgccgcaggtgggtgctgccctgtgtacatataaatgaatctggtgttggggaaaccttcatctgaaacccacagatgtctctggggcagatccccactgtcctaccagttgccctagcccagactctgagctgctcaccggagtcattgggaaggaaaagtggagaaatggcaagtctagagtctcagaaactcccctgggggtttcacctgggccctggaggaattcagctcagcttcttcctaggtccaagccccccacaccttttccccaaccacagagaacaagagtttgttctgttctgggggacagagaaggcgcttcccaacttcatactggcaggagggtgaggaggttcactgagctccccagatctcccactgcggggagacagaagcctggactctgccccacgctgtggccctggagggtcccggtttgtcagttcttggtgctctgtgttcccagaggcaggcggaggttgaagaaaggaacctgggatgaggggtgctgggtataagcagagagggatgggttcctgctccaagggaccctttgcctttcttctgccctttcctaggcccaggcctgggtttgtacttccacctccaccacatctgccagaccttaataaaggcccccacttctcccattaaaaaaaaaaaaaaaaaaaaaaaaaaaaaaaaaaaaaaaa

SEQ ID NO: 7 - DNA, 3352 bp, Homo sapiens
ggaatgttagcgggtgggaggtgcggctgggttgctacagccagagctgggcggtggcgggcgctgctgaaggagtctcgctgagctcgaggaggtggcgcgatggagggactggaagagaatggaggtgttgtccaagttggagaattgttaccttgcaagatttgtggaagaacattctttccagtagcattaaaaaaacatggacccatttgccagaagactgcaactaaaaaacggaagacttttgattcaagcagacagagagctgaaggaactgatattccaacagtaaaacctctcaaaccgaggccagaaccaccaaagaaaccatctaattggagaaggaaacatgaagaattcattgctaccataagagcagctaaaggccttgatcaggccctcaaagagggtggcaaacttcctcctcctcctccaccttcttatgatcctgattatattcaatgtccatattgtcagaggagattcaatgaaaatgcagctgatagacatataaatttctgtaaagaacaggcagcacgtattagtaataaagggaaattttctacagataccaaaggaaaaccaacttctcggacacaggtgtataagccacccgcacttaaaaagtcaaattctcctggaactgcatcatcaggatcttcacgattaccgcagccaagtggcgctggcaaaactgttgtaggtgttccttcaggtaaagtgtcttcaagtagcagctctttgggaaacaaacttcagaccttatctccctctcataaagggatagcagcccctcatgcaggagctaatgtcaaaccccgaaattccacaccacctagtttggcaagaaatcctgccccaggtgtgcttacaaacaaaagaaaaacatatactgagagctacatagccaggccagatggggactgtgcatcttcccttaatggtggaaatattaaaggcattgaaggacattcacctggaaacttaccaaaattctgccatgagtgtgggactaaataccctgtagaatgggccaaattttgctgtgaatgtggcattcgaagaatgattctatgaatagaatctcaaaaaaaaaaaaaagccaagttcagagtttatgattattgctgcttggacagctagagcacatcctctagttagtttgtgctaaaaatactcgaaataccatttccagttaattttgaagtgtaatcttttggctatataatgtgtgtatgtttatatgtgtacatatactgtatataataaatatctgataccccagactgtgtcattcaaggaaatattcacttatctgtcagaaaataatttcaaatggaacaattaatttaggtgttacttttctgttgttgttaaagcacgttaagtacatttccacaaactctcatactctagtgcttagcccttcaagctttaggataagtataactttgagcaaaaaatttggataaacatttgtattattggtgcctatcttcagatagtatcatagtaggatagatgctgggaattttgtaattaaagagaacagttttagttaccattaggtatgttaaggtcactcctgtagagcatgtcaacaaattatttcataaatccgaaaaactaagagaaaaaaaaacccctctattagttacgtaatttaaacatcttacttgttttgtaaaagtgtaattatgcaatttccttttcaaatacacaagactaaaaatacaaatctatcttgttctatttataactgattttttttagttctgtatgtattcgttgaacatatttttttacttccaattgtttttccagatttcacttttcctctttttccataacccgtgaagatagttttaatttgctagttcattttttgctgtatttgaaaatgtaattatttaatatagaaggcacagaattccctgcaaaatcttacatgtttaaaaacatataattttttgtatctgaatttagaataaatcattttaatgcatcttaaatcacgtcacctaatcataatagttgtggtaactaatcattagtgcagagcatgcagataaaaaatatttgaatttttttttcttggaagtacatgtagttatgagtaggttaagagaatatctaattttcctactctttttttcatctttagcatttaatgttgaaacaccattcacgtcacattaaggacccactgaaaatgtattgttttaatgcataattgacttacctaattaaacatacacacaaatttaattatttttttctctttaacctgatcacgacacagctcttaatcattgtcactttcagtatatcatagtagttagtagtagattgccttgatggcagtaattctgtagtaatttctattcaatcaaacctaagagagctttgatcttactgtaaaggtacaaacaaatctcttatataattcctagcttttttttttgtttatgattctgatcaactataagacacaatgtaaagaattgtggcttataatttatctgaaatttattagcttagtttagtttgggcaggagttacaaacttaataggaattgtcattttacttacagtttatttcctgattagttgttaatttttttcccctcagttatctttattcagtcttattggtcaaaacaaagaaacaaatagtccatcaactaaaataagctgtaatgaatttagaagatgaatgcatatattagatttccatttaaatcacttctgttataaatcatataaagaactttaaacttgttttatctaatactgagcactgtttttttgtcaagtatttttttaagaccacataattctttttgtctgctcaaggaaaggatagataaataattggcacacatttgtttctcactgaattttacagtagtaaattaatgttataatgtaccacatggagatgagttggtaagaaatcatctagttccagagcccagagattataaacagtaggtgaaatagatttatgacttacgaaatatgttgtgacaatatatttaaatgcatttttatattacttgcatattctcacattgatttgtgcaatagttcagttttaaaaaaaatcttcctatgcatcatgtattttatttttatttattttcacaagtatttgacagtatggtagaataaaagatgattgtaagattaaaaatgtaaaaattgcttatgtattattctgaattgtgttaggttgaaaaagatgattgtggtgactattatttcttgtccactatttgttttttgttttttcaccaataatgtcttcatatttgaacctattcaataaagacatgaagcataaaaa

SEQ ID NO: 8 - DNA, 2222 bp, Homo sapiens
ctaaagttctgaaagacctgttgcttttcaccaggaagttttactgggcatctcctgagcctaggcaatagctgtagggtgacttctggagccatccccgtttccccgccccccaaaagaagcggagatttaacggggacgtgcggccagagctggggaaatgggcccgcgagccaggccggcgcttctcctcctgatgcttttgcagaccgcggtcctgcaggggcgcttgctgcgttcacactctctgcactacctcttcatgggtgcctcagagcaggaccttggtctttccttgtttgaagctttgggctacgtggatgaccagctgttcgtgttctatgatcatgagagtcgccgtgtggagccccgaactccatgggtttccagtagaatttcaagccagatgtggctgcagctgagtcagagtctgaaagggtgggatcacatgttcactgttgacttctggactattatggaaaatcacaaccacagcaaggagtcccacaccctgcaggtcatcctgggctgtgaaatgcaagaagacaacagtaccgagggctactggaagtacgggtatgatgggcaggaccaccttgaattctgccctgacacactggattggagagcagcagaacccagggcctggcccaccaagctggagtgggaaaggcacaagattcgggccaggcagaacagggcctacctggagagggactgccctgcacagctgcagcagttgctggagctggggagaggtgttttggaccaacaagtgcctcctttggtgaaggtgacacatcatgtgacctcttcagtgaccactctacggtgtcgggccttgaactactacccccagaacatcaccatgaagtggctgaaggataagcagccaatggatgccaaggagttcgaacctaaagacgtattgcccaatggggatgggacctaccagggctggataaccttggctgtaccccctggggaagagcagagatatacgtgccaggtggagcacccaggcctggatcagcccctcattgtgatctgggagccctcaccgtctggcaccctagtcattggagtcatcagtggaattgctgtttttgtcgtcatcttgttcattggaattttgttcataatattaaggaagaggcagggttcaagaggagccatggggcactacgtcttagctgaacgtgagtgacacgcagcctgcagactcactgtgggaaggagacaaaactagagactcaaagagggagtgcatttatgagctcttcatgtttcaggagagagttgaacctaaacatagaaattgcctgacgaactccttgattttagccttctctgttcatttcctcaaaaagatttccccatttaggtttctgagttcctgcatgccggtgatccctagctgtgacctctcccctggaactgtctctcatgaacctcaagctgcatctagaggcttccttcatttcctccgtcacctcagagacatacacctatgtcatttcatttcctatttttggaagaggactccttaaatttgggggacttacatgattcattttaacatctgagaaaagctttgaaccctgggacgtggctagtcataaccttaccagatttttacacatgtatctatgcattttctggacccgttcaacttttcctttgaatcctctctctgtgttacccagtaactcatctgtcaccaagccttggggattcttccatctgattgtgatgtgagttgcacagctatgaaggctgtacactgcacgaatggaagaggcacctgtcccagaaaaagcatcatggctatctgtgggtagtatgatgggtgtttttagcaggtaggaggcaaatatcttgaaaggggttgtgaagaggtgttttttctaattggcatgaaggtgtcatacagatttgcaaagtttaatggtgccttcatttgggatgctactctagtattccagacctgaagaatcacaataattttctacctggtctctccttgttctgataatgaaaattatgataaggatgataaaagcacttacttcgtgtccgactcttctgagcacctacttacatgcattactgcatgcacttcttacaataattctatgagataggtactattatccccatttcttttttaaatgaagaaagtgaagtaggccgggcacggtggctcacgcctgtaatcccag

SEQ ID NO: 9 - DNA, 2656 bp, Homo sapiens
gcatttcccctcggctgccggcggctccgacatcatgctccggctcctccggccgctgctgctactgctgctgctgcctcccccggggtcccctgagccccccggcctgacccagctgtccccgggggcgcccccgcaggcccccgacttgctctacgctgacgggctgcgcgcctacgcggccggggcttgggcgccggccgtggcgctgctgcgggaggcgctgcggagccaggcggcgctgggccgggtgcggctggattgcggggcgagctgcgcggccgatccgggcgccgcgctccccgccgtgcttctcggggccccggagcccgactccgggccgggacccacgcaggggtcctgggagcgacagcttctccgtgcagcgctccgccgcgcagactgcctgacccagtgcgcagcacggaggctgggccccgggggcgcggcgcggcttcgcgtggggagcgcgctccgggacgccttccgccgtcgggagccctacaactacctgcagagggcctattaccagttgaagaagctggatctggcagctgcggcagcacacaccttctttgtagcaaaccccatgcacctgcagatgcgggaggacatggctaagtacagacgaatgtcgggagttcggccccagagcttccgggacctggagacgcccccacactgggcagcctatgacactggcctggagctactggggcgccaggaggcaggactggcactgcccaggctagaggaggctcttcaggggagcctggcccagatggagagctgccgtgctgactgtgaggggcctgaggagcagcagggggctgaagaagaggaggatggggctgcgagccaggggggcctctatgaggccattgcaggacactggattcaggtcctgcagtgccggcaacgctgtgtgggggaaacagccacacgccctggtcgcagcttccctgtcccagacttccttcccaaccagctgaggcggctacatgaggcccatgctcaggtgggcaatctgtcccaggctatagaaaatgtcctgagtgtcctgctcttctacccggaggatgaggctgccaagagggctctgaaccagtaccaggcccagctgggagagccgagacctggcctcggacccagagaggacatccagcgcttcatcctccgatccctgggggagaagaggcagctctactatgccatggagcacctggggaccagcttcaaggatcctgacccctggacccctgcagctctcatccctgaggcacttagagaaaagctcagagaggatcaagagaagaggccttgggaccatgagcccgtgaagccaaagcccttgacctactggaaggatgtccttctcctggagggtgtgaccttgacccaggattccaggcagctgaatgggtcggagcgggcggtgttggatgggctgctcaccccagccgagtgtggggtgctgctgcagctggctaaggatgcagctggggctggagccaggtctggctatcgtggtcgccgctcccctcacaccccccatgaacgcttcgaggggctcacggtgcttaaggctgcgcagctggcccgggctgggacagtgggcagtcagggtgctaagctgcttctggaggtgagcgagcgggtgcggaccttgacccaggcctacttctccccggaacggcccctgcatctgtccttcacccacctggtgtgccgcagcgccatagaaggagagcaagagcagcgcatggacctgagtcacccagtgcacgcagacaactgcgtcctggaccctgacacgggagagtgctggcgggagcccccagcctacacctatcgggactacagcggactcctctacctcaacgatgacttccagggtggggacctgttcttcacggagcccaacgccctcactgtcacggctcgggtgcgtcctcgctgtgggcgccttgtggccttcagctccggtgtcgagaatccccatggggtgtgggccgtgactcggggacggcgctgtgccctggcactgtggcacacgtgggcacctgagcacagggagcaggagtggatagaagccaaagaactgctgcaggagtcacaggaggaggaggaagaggaagaggaagaaatgcccagcaaagacccttccccagagccccctagccgcaggcaccagagggtccaagacaagactggaagggcacctcgggttcgggaggagctgtgagtggctgagccagctccttgaggatgtggccacttgacttgtggaaggccatcttgatgccaggacacacaggaagcccctgtgtgacatcaggagcagaacagcaagctctctgtccctgcacccccaccatcttggggacctacaagggcctggactcagaggacagtgcacaggctagcctggagctcaccaggcctggggagctgggacggggccccgctgccggacctgcagccctggacagatggggaacactgtgcctccctgaacagaaatggcaggggaggaggctgatgctttaaatgaagaggatggtggggttgggaggtataaccctgctcctctctcccagtctgtgcaataaaggtcgtgaagatctctcagccagggaaaaaaaaaaaaaaaaaa

SEQ ID NO: 10 - DNA, 3963 bp, Homo sapiens
cctgcgtgtccctctgcgctccgactggtgcgacttctccctgcgctagcgaggcagggttttggcctcgcctctcgcgagatcgcctcctgttgctgccgccgccgctcctggccactgactggcggcgcctgcgcagccgccatgttcggttgctatgctgcggcctaggagagggggtgtgcttgagggaggaggaagagatagaggaggaggagggggaggaagaggaggtggagaaggaggggggtgactgagctcctcttgcactctcacacacaaacgctgcccaggattacccgccagctcacgccgcgcagtgcgcttttccgctcctcgcgccccaccaccaacattgttctctcaggactcctgggtcccaggggtcggaattgggcctgagcgggagaggaaagagacttggctttggccgcggggtcggaggattggggccaggccccctcccccacgcacttttgggggtgtggattatctcatccctgcagggaggtaggagaggtcgccggctgcccgcctccctgccacctccccagcggcgccggcccgcggctgcccagcagcatgaggtggtgctggcggctccgggtcgtggcgcgaccgctgcggcggcggctgctcggggggcgctgaggtagccccccggagcggcacggaggacgcgcttctcctctgcgcgccggggcctcgaggctttttttctccagccgagaggacgcggctgtgatatacgaagactttgtgtggacagtaatgacctcacgtttccgattgcctgctggcagaacctacaatgtacgagcatcagagttggcccgagacagacagcatactgaagtggtttgcaacatccttcttctggataacactgtacaagctttcaaagtcaataaacatgatcaggggcaagtcttgttggatgtcgtcttcaagcatctagatttgactgagcaggactattttggtttacagttggctgatgattccacagataacccaaggtggctggatccaaacaaaccaataaggaagcagctaaagagaggatctccttacagtttgaactttagagtcaaattttttgtaagtgaccccaacaagttacaagaagaatatacaaggtaccagtattttttgcaaattaaacaagacattcttactggaagattaccctgtccttctaatactgctgcccttttagcttcatttgctgttcagtctgaacttggagactacgatcagtcagagaacttgtcaggctacctctcagattattctttcattcctaatcaacctcaagattttgaaaaagaaattgcaaaattacatcagcaacacataggcttatctcctgcagaagcagaatttaattacctaaacacagcacgtaccttagaactctatggagttgaattccactatgcaagggatcagagtaacaatgaaattatgattggagtgatgtcaggaggaattctgatttataagaacagggtacgaatgaatacctttccatggttgaagattgtaaaaatttcttttaagtgcaaacagttttttattcaacttagaaaagaattgcatgaatctagagaaacattattgggatttaatatggtgaattacagagcatgtaaaaatttgtggaaagcatgtgtagaacatcacacattcttccgtttggacagaccacttccacctcaaaagaatttttttgcacattattttacattaggttcaaaattccggtactgtgggagaactgaagtccaatcagttcagtatggcaaagaaaaggcaaataaagacagggtatttgcaagatccccaagtaagcccttggcacggaaattaatggattgggaagtagtaagcagaaattcaatatctgatgacaggttagaaacacaaagtcttccatcacgatctccaccgggaactcctaatcatcgaaattctacattcacgcaggaaggaacccggttacgaccatcttcagttggtcatttggtagaccatatggttcatacttccccaagcgaagtgtttgtaaatcagagatctccgtcatcaacacaagctaatagcattgttctggaatcatcaccatcacaagagacccctggagatgggaagcctccagctttaccacccaaacagtcaaagaaaaacagttggaaccaaattcattattcacattcgcaacaagatctagaaagtcatattaatgaaacatttgatattccatcttctcctgaaaaacccactcctaatggtggtattccacatgataatcttgtcctaatcagaatgaaacctgatgaaaatgggaggtttggattcaatgtaaagggaggatatgatcagaagatgcctgtgattgtgtctcgagtagcaccaggaacacctgctgacctctgtgtccctagactgaatgaaggggaccaagttgtactgatcaatggtcgggacattgcagaacacactcatgatcaggttgtgctgtttattaaagctagttgtgagagacattctggggaactcatgcttctagttcgacctaatgctgtatatgatgtagtggaagaaaagctagaaaatgagccagatttccagtatattcctgagaaagccccactagatagtgtgcatcaggatgaccattccctgcgggagtcaatgatccagctagctgaggggcttatcactggaacagtcctgacacagtttgatcaactgtatcggaaaaaacctggaatgacaatgtcctgtgccaaattacctcagaatatttccaaaaatagatacagagatatttcgccttatgatgccacacgggtcattttaaaaggtaatgaagactacatcaatgcgaactatataaatatggaaattccttcttccagcattataaatcagtacattgcttgtcaagggccattaccacacacttgtacagatttttggcagatgacttgggaacaaggctcctctatggttgtaatgttgaccacacaagttgaacgtggcagagttaaatgtcaccaatattggccagaacccacaggcagttcatcttatggatgctaccaagttacctgccactctgaagaaggaaacactgcctatatcttcaggaagatgaccctatttaaccaagagaaaaatgaaagtcgtccactcactcagatccagtacatagcctggcctgaccatggagtccctgatgattcgagtgactttctagattttgtttgtcatgtacgaaacaagagggctggcaaggaagaacccgttgttgtccattgcagtgctggaatcggaagaactggggttcttattactatggaaacagccatgtgtctcattgaatgcaatcagccagtttatccactagatattgtaagaacaatgagagatcagcgagccatgatgatccaaacacctagtcaatacagatttgtatgtgaagctattttgaaagtttatgaagaaggctttgttaaacccttaacaacatcaacaaataaataagaaagcaaaaagatctgggatatgtgttggaaaactgctttcccttatgttcactgtgccataatgctgctcgcaggaaatggcattttacaaaaaaaaaatgaagaactcaaaaaaactttgaaaacttcagcactgttgcactttatgttttaaaaaatgtcactctttcaaaatctataactcatgtatttgaagactgtttcatgctttgctccgaacaaatagtaaataactgagtatgttcagggtaatttatgaaattttgtggtggtgccatgcaatccccttttggtagaattgccacaaacaaggctcaaaattctcatcatctctgttatacacctgtatcatgaaagcaaaaagaagtaaacatcaggagtcagctctgaaaaaaaaaaaaaaaaaaaa

SEQ ID NO: 11 - DNA, 5820 bp, Homo sapiens
agccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacaggatccctgctgggcaccaacagctccaccatggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattgatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcacctgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttcctgaattatcatggagttttctaattctctcttttggaatgtagattttttttaaatgctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattctttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagatatctcagattgtgtagatatgctatttaaataatttatcaggaaatactgcctgtagagttagtatttctatttttatataatgtttgcacactgaattgaagaattgttggttttttcttttttttgttttgtttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatatttataaaaaaaaaaaaaaaaaaaaaaaaaaaaaaa

SEQ ID NO: 12 - DNA, 2330 bp, Homo sapiens
cttggtcccgccccgggcccctggctcctggccccgccccagcccaggcaggtcactgcgccatttcctgtccaaagctgggcgaatcagggattccggttcacaatggatgctgataaagagaaagatttgcagaaatttcttaaaaatgtggatgaaatctccaatttaattcaggagatgaattctgatgacccagttgtgcaacagaaagctgtcctggagacagaaaagagactactgcttatggaggaagaccaggaggaggatgaatgcaggaccaccttgaacaagactatgatcagtcctccacaaactgctatgaagagtgcagaagaaataaactcagaggccttcttggcatctgtggagaaggatgcaaaggaacgagccaagagaagaagggaaaacaaagtcttggcggatgccctaaaagaaaaagggaatgaagcatttgctgaaggcaattatgaaacagctatcctgcgctacagtgagggtttggagaagctgaaggacatgaaagtgctgtacaccaaccgagcccaggcttatatgaaacttgaggactatgagaaggcactggtggattgtgagtgggctctcaagtgtgatgaaaaatgcacaaaagcatattttcacatgggaaaagccaacctggccctgaagaactacagtgtgtctagagagtgttataagaagatcttagaaataaaccccaagctgcaaacccaggtgaaaggttacctgaatcaagtagatcttcaggaaaaagcagaccttcaagaaaaggaagcccacgaactgctggattcaggaaagaacacagccgtgaccaccaagaacctcctggagaccctttccaagcctgaccagatccccttgttctatgctggggggattgagatcctgactgaaatgataaatgagtgcacagaacaaactttattcagaatgcacaatggatttagtatcatcagtgacaacgaggtcataagaaggtgtttttccacagcaggaaatgatgcagttgaagaaatggtctgtgtgtctgttctcaagctctggcaagcagtgtgcagcaggaacgaggaaaaccagcgtgtgctagtgatacaccatgacagggccaggctgttggccgccctcttgtcctccaaggtcctggccatccggcagcagagctttgccctgctgctgcatctcgcccagactgagagcggacggagcctgatcatcaaccaccttgacctgaccagattattggaagcgctggtgtcatttcttgatttctcggataaggaggccaacactgctatgggactgttcacagacttggctctggaagaaagattccaagtctggttccaggccaaccttccaggtgttctccctgcactcacaggcgttctgaagacagatcccaaggtaagcagctcctcggctctgtgccagtgcattgccatcatgggaaacctcagtgctgagcccactacccgaagacacatggcggcctgtgaggaatttggggatggctgcttgagcctcctggccaggtgtgaggaggatgtggacctgttcagagaggttatctacacactcctgggactcatgatgaacctgtgtcttcaggctccctttgtctctgaggtttgggctgtggaggtgagcagaaggtgcctgtctttactaaacagccaggatggaggaatcctgacaagagctgctggtgttctgagccggaccctttcttcctctctgaaaattgttgaggaggccttgcgagcaggagtggtaaagaaaatgatgaaattcctgaagacaggaggtgagactgcatcacgttatgctataaagatactagctatctgcacgaatagttatcatgaagctcgggaagaagtaataagactggataaaaagttgagcgttatgatgaagctgctcagctcggaggatgaggttctggtgggcaacgctgccctctgccttggtaactgcatggaggtgcccaacgttgcgtcttccctgctaaagacggaccttttgcaggtcttgttaaagcttgcaggcagtgacacacagaagacggccgtgcaggtgaacgcaggcattgctctggggaagctgtgcacagctgagcccagatttgctgctcaactgagaaagcttcatggcctagaaattctcaactctacgatgaaatacatcagtgattcttgagagagacagggtttgtgtgcatttggggaacacacagatgcacaccgtgtgttgttcctatgctaataaagacctttgatgtatccacttcaaaaaaaaaaaaaaaa

SEQ ID NO: 13 - DNA, 2368 bp, Homo sapiens
gcgccgccaggcgtaggcggggtggcccttgcgtctcccgcttccttgaaaaacccggcgggcgagcgaggctgcgggccggccgctgcccttccccacactccccgccgagaagcctcgctcggcgcccaacatggcgggtgggcgctgcggcccgcagctaacggcgctcctggccgcctggatcgcggctgtggcggcgacggcaggccccgaggaggccgcgctgccgccggagcagagccgggtccagcccatgaccgcctccaactggacgctggtgatggagggcgagtggatgctgaaattttacgccccatggtgtccatcctgccagcagactgattcagaatgggaggcttttgcaaagaatggtgaaatacttcagatcagtgtggggaaggtagatgtcattcaagaaccaggtttgagtggccgcttctttgtcaccactctcccagcattttttcatgcaaaggatgggatattccgccgttatcgtggcccaggaatcttcgaagacctgcagaattatatcttagagaagaaatggcaatcagtcgagcctctgactggctggaaatccccagcttctctaacgatgtctggaatggctggtctttttagcatctctggcaagatatggcatcttcacaactatttcacagtgactcttggaattcctgcttggtgttcttatgtgtttttcgtcatagccaccttggtttttggcctttttatgggtctggtcttggtggtaatatcagaatgtttctatgtgccacttccaaggcatttatctgagcgttctgagcagaatcggagatcagaggaggctcatagagctgaacagttgcaggatgcggaggaggaaaaagatgattcaaatgaagaagaaaacaaagacagccttgtagatgatgaagaagagaaagaagatcttggcgatgaggatgaagcagaggaagaagaggaggaggacaacttggctgctggtgtggatgaggagagaagtgaggccaatgatcaggggcccccaggagaggacggtgtgacccgggaggaagtagagcctgaggaggctgaagaaggcatctctgagcaaccctgcccagctgacacagaggtggtggaagactccttgaggcagcgtaaaagtcagcatgctgacaagggactgtagatttaatgatgcgttttcaagaatacacaccaaaacaatatgtcagcttccctttggcctgcagtttgtaccaaatccttaatttttcctgaatgagcaagcttctcttaaaagatgctctctagtcatttggtctcatggcagtaagcctcatgtatactaaggagagtcttccaggtgtgacaatcaggatatagaaaaacaaacgtagtgttgggatctgtttggagactgggatgggaacaagttcatttacttaggggtcagagagtctcgaccagaggaggccattcccagtcctaatcagcaccttccagagacaaggctgcaggccctgtgaaatgaaagccaagcaggagccttggctcctgagcatccccaaagtgtaacgtagaagccttgcatccttttcttgtgtaaagtatttatttttgtcaaattgcaggaaacatcaggcaccacagtgcatgaaaaatctttcacagctagaaattgaaagggccttgggtatagagagcagctcagaagtcatcccagccctctgaatctcctgtgctatgttttatttcttacctttaatttttccagcatttccaccatgggcattcaggctctccacactcttcactattatctcttggtcagaggactccaataacagccaggtttacatgaactgtgtttgttcattctgacctaaggggtttagataatcagtaaccataacccctgaagctgtgactgccaaacatctcaaatgaaatgttgtggccatcagagactcaaaaggaagtaaggattttacaagacagattaaaaaaaaattgttttgtccaaaatatagttgttgttgatttttttttaagttttctaagcaatatttttcaagccagaagtcctctaagtcttgccagtacaaggtagtcttgtgaagaaaagttgaatactgttttgttttcatctcaaggggttccctgggtcttgaactactttaataataactaaaaaaccacttctgattttccttcagtgatgtgcttttggtgaaagaattaatgaactccagtacctgaaagtgaaagatttgattttgtttccatcttctgtaatcttccaaagaattatatctttgtaaatctctcaatactcaatctactgtaagtacccagggaggctaatttcttt

SEQ ID NO: 14 - DNA, 6462 bp, Homo sapiens
ggtcccgccccttcctaccttccagtagccggcggcggtgtctcaggcggcaatggaaggatccgagcctgtggccgcccatcagggggaagaggcgtcctgttcttcctgggggactggcagcacaaataaaaatttgcccattatgtcaacagcatctgtggaaatcgatgatgcattgtatagtcgacagaggtacgttcttggagacacagcaatgcagaagatggccaagtcccatgttttcttaagtgggatgggtggtcttggtttggaaattgcaaagaatcttgttcttgcagggattaaggcagttacaattcatgatacagaaaaatgccaagcatgggatctaggaaccaacttctttctcagtgaagatgatgttgttaataagagaaacagggctgaagctgtacttaaacatattgcagaactaaatccatacgttcatgtcacatcatcttctgttcctttcaatgagaccacagatctctcctttttagataaataccagtgtgtagtattgactgagatgaaacttccattgcagaagaagatcaatgacttttgccgttctcagtgccctccaattaagtttatcagtgcagatgtacatggaatttggtcaaggttattttgtgatttcggtgatgaatttgaagttttagatacaacaggagaagaaccaaaagaaattttcatttcaaacataacgcaagcaaatcctggcattgttacttgccttgaaaatcatcctcacaaactggagacaggacaattcctaacatttcgagaaattaatggaatgacaggtttaaatggatctatacaacaaataacggtgatatcgccattttcttttagtattggtgacaccacagaactggaaccatatttacatggaggcatagctgtccaagttaagactcctaaaacagttttttttgaatcactggagaggcagttaaaacatccaaagtgccttattgtggattttagcaaccctgaggcacctttagagattcacacagctatgcttgccttggaccagtttcaggagaaatacagtcgcaagccaaatgttggatgccaacaagattcagaagaactgttgaaactagcaacatctataagtgaaaccttggaagagaagcctgatgtaaatgctgacattgtgcattggctctcttggactgcccaaggctttttatctccacttgctgcagcagtaggaggtgttgccagccaagaagtattgaaagctgtaacaggaaaattttctcctttgtgccagtggttatatcttgaagcagcagatattgttgaatcactaggcaaacctgaatgtgaagaatttctcccacgaggagatagatatgatgccttaagagcttgcattggagacactttgtgtcagaaactgcaaaatttaaacatcttcttagtagggtgtggagccataggctgtgaaatgttgaaaaattttgctttacttggtgttggcacaagcaaagagaaaggaatgattacagttacagatcctgacttgatagagaaatccaacttaaatagacagttcctatttcgtcctcatcacatacagaaacctaaaagctacactgctgctgatgctactctgaaaataaattctcaaataaagatagatgcacacctgaacaaagtatgtccaaccactgagaccatttacaatgatgagttctatactaaacaagatgtaattattacagcattagataatgtggaagccaggagatacgtagacagtcgttgcttagcaaatctaaggcctcttttagattctggaacaatgggcactaagggacacactgaagttattgtaccgcatttgactgagtcttacaatagtcatcgggatcccccagaagaggaaataccattttgtactctaaaatcctttccagctgctattgaacataccatacagtgggcaagagataagtttgaaagttccttttcccacaaaccttcattgtttaacaaattttggcaaacctattcatctgcagaagaagtcttacagaagatacagagtggacacagtttagaaggctgttttcaagttataaagttacttagcagaagacctagaaattggtcccagtgtgtagaattagcaagattaaagtttgaaaaatattttaaccataaggctcttcagcttcttcactgtttccctctggacatacgattaaaagatggcagtttattttggcagtcaccaaagaggccaccctctccaataaaatttgatttaaatgagcctttgcacctcagtttccttcagaatgctgcaaaactatatgctacagtatattgtattccatttgcagaagaggacttatcagcagatgccctcttgaatattctttcagaagtaaagattcaggaattcaagccttccaataaggttgttcaaacagatgaaactgcaaggaaaccagaccatgttcctattagcagtgaagatgagaggaatgcaattttccaactagaaaaggctattttatctaatgaagccaccaaaagtgaccttcagatggcagtgctttcatttgaaaaagatgatgatcataatggacacatagatttcatcacagctgcatcaaatcttcgtgccaaaatgtacagcattgaaccagctgaccgtttcaaaacaaagcgcatagctggtaaaattatacctgctatagcaacaaccactgctacagtttctggcttggttgccttggagatgatcaaagtaactggtggctatccatttgaagcttacaaaaattgttttcttaacttagccattccaattgtagtatttacagagacaactgaagtaaggaaaactaaaatcagaaatggaatatcatttacaatttgggatcgatggaccgtacatggaaaagaagatttcaccctcttggatttcataaatgcagtcaaagagaagtatggaattgagccaacaatggtggtacagggagtcaaaatgctttatgttcctgtaatgcctggtcatgcaaaaagattgaagttaacaatgcataaacttgtaaaacctactactgaaaagaaatatgtggatcttactgtgtcatttgctccagacattgatggagatgaagatttgccgggacctccagtaagatactacttcagtcatgacactgattaatacaagttgtcttaacgttactccaggaccacttgattttggaaagagtgcacttaattcagaagctaaagaaaatcagttcataatactatggatttctctttcattaagccttaattttaagggaaacatcagtaagaaactgcactgaagaattataaaacattttggggcatagcatacacttgtctaacggttcacacgtggctatgatcacaagcaactttgaactggaatgctatttacaaaagttttgtgtattaatctgtgtattaatctctctggataaaaagaaggaaaaaatatgtatgaccagaacagatatggatgaagaaattgaaagcaacgaatgcaactattcaaaaagtttaattttatgaatttcttttttgtttagtcttgaagactgattttctatgcaaatagtgtttggcatcctgcacctctgatatgatttggctttgagaatttaataccactgggaagaagtatggtagtggtggatgaagggtggatattttaaattgtgcagttacagtttactgtcctattacctctgctcgtttaaccagtttgttatatcactgtgtccccaaaatcaggatttttgttgatagcatcagtgttgtaggagcaataggtcagatgagacatattaacttagactaaacgtgaacagtattatatggactctcacaacgctcttagagaatccgtgaatgtgaacagacaaatgtggctaaccatttgattcttcagtatgccttctaatgtggctattttatttatgtgagactctaaacctgattgtcctaatatataaaactaaaagattttgtaaagggagtgtctttagaaatagatgaaatgtagaatgttaaaaattattgctagggtagtcttttttttttccagaaacctaattagggtattaaattttgtgttttttttgttttttttttttaaacagaagcatgttatttcattcccattcccagaaagggagttaatgaagataaaaatttattttttaaggtctttattgagagaaactttgttttctgatatgaactattgcagatgtttttataaatactttcattaaaatgatgtaaacagtagtacccaacactgtaaactcagtgaaaatagtaaatgattcttttattactaagactgtcatgcattctgaagcagttggcttttttttaaccataggaagtaatttccctctagctcctttccttctactctcctgctcagaccattagtaggtactttgttaaataaaaaactagattaacatcaatattactccaatttggtatcttttacactatgtattatacctactttctttttatttcatttacaaatagtttaaattactttatcaaccagctgtattgtttccctcttgtaaaagtaccatcaagtggggaaaatgtatgtggaagtggagagtgaatttgtatgactaaaggataatctgtacatggggaagtgggcaaaagtggataggatgaatttaaagaaaatgactacctttggaaaaaagaaattaaattttgttcacatatcctaccctttcccattgtgcatatcccaagtgtcatatttaaaactaaggttacttaaaacagaatccaggaatatcaaggctctgtggcttggaattttagaggataggactaataaaaggacttttgcaaagaaggcttttttccacgttcactttgttttgtgttctttgaaagtaactgatacttttcgggtagttaattcagcagtccataaatatgatccagtaacttgcttatattttattgaagtctcgacagctcttcagaagtaaatttagaacgatgctgtcagttcatatttatagatattagtgttttagcagataaaacaaaatcaacaaaaattaagttcattttgtgattaaacctgcaaccatttttccattactttttttctatagttaatggttattgccatgatttcttctgtttggttctactaagctagaaagccagggtgaagttaatgataattcccattattttatttctgtaccatgagattgctgttgatgactgaaataccaggtgcaaaaattaatgatttgatttttgtacagtttcaatgagtattttttacttattaaaaataaattaagaaatgtaagaatatctttgtaaatcatgtttcataagttgactccagagattctgattttgctgtttattttgtgagtaatgttgctttggtgtttcctgattttcaagtttgcaatcatggagatacagcagttattaggtgtggaaggacattacctcaaatgtcctcagatggctccaggaaattcttttaaaacagttggagagaaatagtcgtgaccatgtaaaacctagaggatctggtaaatcccatactactggttagggatttgtaaagtccatttctttttagagttcaaagcagttctgttacttgtccataagttcctcataaattgtcccaaaggtaggacatggaaataaatgtatatctttattttttaatctactatgtcacactctggtgatattcatggaacattaaaaaagcaactatgtttattttataattagtaaaatgaagatagaaaattgttcttttgaaatttttagggaaaaaaaaaatctcgtgacctaaatctttccactataatgaatattcattgtgattgcaaatatctaattttatataatcactgtgtatggatttagcgattttacttttcatagattcaaaaacatataatctgaatgctttctctgaaaaaaaaaaaatcagagttgagctcttgaggatcagagttaagtatccattgataatatgtgttcattttgctctatgtcataactttgtttttttattgagaaatatatatttttcaaaaacacatatatattcaaaaatacataaaatataaatatgcagttcaaggaataattttaaagcaactatataactagcactcagcttagtagaatatattgacagcagttcagaaattacacatgtagttccttcccagtcacaaccagctccctgccacgtaccctttggtaattaccactatcctaccttgtatgataattgtctctttgtttttatagtgttaccacctatgtgtgtaaacttaaacaatacagtttgcttgtttttgagttttatttaaaatcataaaaaaaaaaaaaaaa

SEQ ID NO: 15 - DNA, 3632 bp, Homo sapiens
gtagtcttttttttgcagaatgggccaaggggacagagacttcactcgggagccagaacctgttttcaacatgggtattttaagaagagacggagaggctgtcactgcagtggtcactgtcctggaaaagtaaccgttgcacggcccagaagtggctctgtggcggccaatgggagccgaagcctgcgaggatgttgggaattgtagttctggcgtcggtcggtggccgagatcccgggcacgctggctctggtccaccttctccaatccctgcctgctgggagaggacgatctcttgagaaaggaaagacttctgtgctcccgagaacttcctatcaggtcctggctgcagggaaacaagctgggctttttataattaaggttggaagaagtcaccacaggcagcagaactccatcttgagatgaaataacatctacctggacctctggcagaatttcaaggcacacactgggctgactctggcgccatgatgttgccttatccttcagcactgggagatcaatactgggaagagattttgcttccaaagaatggggaaaatgtagagactatgaagaaattgacccaaaatcataaagcgaaaggcttgccttctaatgatactgactgcccccagaaaaaggagggaaaggcccaaatagtggtaccagttacattcagggatgtgactgtgatcttcacagaagcagaatggaagagactgagtccagagcagaggaatctatacaaagaagtgatgctggagaattacaggaatcttctctcattggcagaaccaaagccagaaatctacacttgttcctcctgccttctggccttctcctgtcagcagttcctcagtcaacatgtacttcagatcttcctgggcttatgtgcagaaaatcacttccatccagggaattctagcccagggcattggaaacagcaggggcagcagtattcccatgtaagctgttggtttgaaaatgcagaaggtcaggagagaggaggtggctccaaaccctggtctgcaaggacagaggagagagaaacctcaagggcattccccagcccactccaaagacagtcagcaagtcctagaaaaggcaacatggtggtagaaacagagcccagctcagcccaaagaccaaaccctgtgcagctagacaaaggcttgaaggaattagaaaccttgagatttggagcaatcaactgtagagagtatgaaccggaccataacctggaatcaaactttattacaaacccgaggaccctcttagggaagaagccctacatttgcagtgattgtgggcgaagctttaaagatagatcaaccctcatcagacaccatcgtatacactcgatggagaagccttatgtgtgcagtgagtgcgggcgaggttttagccagaagtccaacctcagcagacaccagagaacacattcagaagagaagccttatttgtgcagggagtgtgggcaaagctttagaagtaagtccatcctcaatagacatcagtggactcactcagaggagaagccctatgtttgcagcgagtgtgggcgaggctttagcgagaagtcatccttcatcagacaccagaggacacactccggtgagaaaccctatgtgtgcctggagtgtggacgaagcttttgtgataagtcaaccctcagaaaacaccagaggatacactcaggggagaagccttatgtttgcagggagtgtgggcgaggctttagccagaactcagatctcatcaaacaccagaggacacacttggatgagaagccttatgtttgcagggagtgtgggcgaggcttttgtgacaagtcaaccctcatcatacacgagcggacgcactctggagagaagccttatgtgtgtggtgagtgtggccgaggctttagtcggaaatcactcctccttgtccaccagaggacacactcaggggagaagcattatgtctgcagggagtgtaggcgaggttttagccagaagtcaaatctcatcagacaccagaggacgcactcaaatgagaagccttatatttgcagggaatgtgggcgaggcttttgtgacaagtcaaccctcattgtacatgagaggacacactcaggagagaagccttacgtgtgcagtgagtgtggccgaggctttagccggaaatcactcctccttgtccaccagaggacacactcaggggagaagcattatgtttgtagggagtgtgggcgaggctttagtcataagtcaaatctcatcagacaccagaggacacactgacgggagaaacctgtgtatgcaggggtcatgaacaagacctgagtgaccagtcaagcctcatgttaccccagagagacacatggggagtagaccctgtgtacacagattgtgagtgaagttccagagatgtgtcagcccttatcaggcatgggagggacacgttcaggagaggagccttatgagtatagagtacgggcaactgtagccatcagtcagccttgagcatgcacaaaaggacacacttaggagagaagtttatgtgtagggactgtgggaaggctttagcaataatcaacatttaccagacatccaatgacagcctcaggggaaagcacccttgtctggggagtgttggggagcatcagtaaaagaatggacactcaggcacagagtggccctcaggaaggaggtctttgtttgtaggatgtatgggcaaagcttttgtgatcacacaccacagggagaatctgcatgtggggacactgtggagctctgcccagatgaccttttcaggggtaacaccccagctgcttgagagaacagtgttgctgctggcagagatgcattccagagatgcactccgctctggaactcactctcagccacagggagctgcatgcaccacaggggcaatgcacctttgcaggggtaccttctggccccaacccttgactcaacggggacaactccagaaggtcattccagatccagagatccccatcgaactgaaggatcactgggttgcagacacattgcaggtcagcttcttcctctgcccagtcctgccctcactccccagtgaatcctcaattttctgtctcgttgtctgtccagataattgattctaagacatgttaggtatataaggagtgtagataaggcttcagccatgagtcaccccccagtaagccccagagtatattgaatagaaattctgcatgtgtggggagaatggacaaggacttaggaaaaagtcctcatcaaagaacagcttttttgggacaagctttacatgggtggggagggaaaatgtgaaacacattagcaataagttaaacctcatcttgtactaaaggaggacacactcagggagaagacctctgtgggcagggcttgtgggtggagcttcatcccgatgtcactcctcaactgacttaggaggacagtcttgctaccccaagtcactacctcactcacctctgagggattttcaggaaatgtcttgactcccccatgtactctgtatgtgagcgaagatggcagtaactgttaaataagcattcttttctacttcttggaatcagatgaaataaaaaagcaggctttatttaagtaatcaaaaaa

SEQ ID NO: 16 - DNA, 1688 bp, Homo sapiens
acttgacagcccgctgaggacgcagcgtcagctgacctggggagtcgcgattcgtgccggccggtcctggttctccggtcccgccgctcccgcagcagccatgtcgttcttcccggagctttactttaacgtggacaatggctacttggagggactggtgcgcggcctgaaggccggggtgctcagccaggccgactacctcaacctggtgcagtgcgagacgctagaggacttgaaactgcatctgcagagcactgattatggtaacttcctggccaacgaggcatcacctctgacggtgtcagtcatcgatgaccggctcaaggagaagatggtggtggagttccgccacatgaggaaccatgcctatgagccactcgccagcttcctagacttcattacttacagttacatgatcgacaacgtgatcctgctcatcacaggcacgctgcaccagcgctccatcgctgagctcgtgcccaagtgccacccactaggcagcttcgagcagatggaggccgtgaacattgctcagacacctgctgagctctacaatgccattctggtggacacgcctcttgcggcttttttccaggactgcatttcagagcaggaccttgacgagatgaacatcgagatcatccgcaacaccctctacaaggcctacctggagtccttctacaagttctgcaccctactgggcgggactacggctgatgccatgtgccccatcctggagtttgaagcagaccgccgcgccttcatcatcaccatcaattctttcggcacagagctgtccaaagaggaccgtgccaagctctttccacactgtgggcggctctaccctgagggcctggcgcagctggctcgggctgacgactatgaacaggtcaagaacgtggccgattactacccggagtacaagctgctcttcgagggtgcaggtagcaaccctggagacaagacgctggaggaccgattctttgagcacgaggtaaagctgaacaagttggccttcctgaaccagttccactttggtgtcttctatgccttcgtgaagctcaaggagcaggagtgtcgcaacatcgtgtggatcgctgaatgtatcgcccagcgccaccgcgccaaaatcgacaactacatccctatcttctagcgtcctggcccaaggctctcaattgcactctttgtgtgtgtgtgtgtgtgtgtgcgcgtgtgtgtgcgtgtgtgtgtatgtggtctgtgacaagcctgtggctcacctgcctgtccggggtgtagtacgctgtcctagcggctgcccagttctcctgaccctcttagagactgttcttaggcctgaaaaggggctgggcacccccccccaccaaggatggacgaagaccccctccagagcaaggaggccccctcagccctgtggttacagccgctgatgtatctaagaagcatgtcactttcatgttcctccctaactccctgacctgagaaccctggggcctgggggcagtttgagcctcctctcccttctgtgggtcgctcccagagccatggcccatgggaaggacagagtgtgtgtgtccttggggcctggggggatgttgctcctcagctccctccctcagccctgcccctctgagacaataaaactgccctctctaaggccaactgtcaaaaaaaaaaaaaaaaaa

SEQ ID NO: 17 - DNA, 2683 bp, Homo sapiens
acatgcgcagtagctgtaggagggggtgggcgctccattgggcgtttctcttggtttttcctttccgcgcggcttgggcggacgtctcgtgagacgtgggacttctcgcgggaactgcattcaaatatcccaggcgcttactcgagagctagctgagcgaatgggccggcgactgtggagttagcgtcctcaatgtggacgccctgagctcccattaggagccgctggctgcggcagcaggggactagcgtgagagttggctaaaaaaaagaaaagaacatggaggcagatataatcacaaatcttcgatgcaggctcaaagaggctgaagaagagcgactaaaagctgcacagtatggtttacaactagtagagagtcaaaatgaattacagaatcaattggataaatgtcgtaatgaaatgatgaccatgactgagagttatgaacaagaaaaatatacccttcaaagagaagttgaactcaagagtcgaatgttagaaagtttgagctgcgaatgtgaagctattaaacaacaacaaaaaatgcacctggagaaattggaagaacaactaagcagaagccatggacaggaagtgaatgaactaaaaactaagatagaaaaactgaaagtggaattagatgaagccaggcttagtgaaaagcagctgaagcaccaagtagatcatcagaaggaactcctctcttgtaaatcagaggaactgcgcgtaatgtctgaacgtgtgcaggaaagcatgtcttcagagatgctggctcttcaaattgagctgacagaaatggagagtatgaagaccaccctcaaagaagaagtgaatgaactacaatacagacaagaacagctagaacttcttattactaacctaatgcgccaggtagaccggcttaaagaggaaaaagaggagcgagagaaagaagcagtttcttactataatgccctagagaaagctcgtgtagcaaatcaagatcttcaggtacagttggaccaggcactccagcaagccttggatcccaatagtaaaggcaactctttgtttgcagaggtggaagatcgaagggcagcaatggaacgtcagctcatcagtatgaaagtcaagtatcagtcactaaagaagcaaaatgtatttaacagagaacagatgcagagaatgaagttacaaattgccacgttgctacagatgaaagggtctcaaactgaatttgagcagcaggaacggttgcttgccatgttggagcagaagaatggtgaaataaaacatcttttaggtgaaattagaaatctggagaaatttaagaatttatatgacagtatggaatctaagccttcagtcgactctggtactctggaagataacacctattatacagatttacttcagatgaagctggataacttaaacaaagaaattgaaagcactaaaggtgaattgtccatacagcgaatgaaagcattatttgagagccagcgggctctagatattgagcgaaaactttttgcaaatgaaagatgcctccagctttcagaaagtgaaaatatgaaactgagagctaaactagatgaattgaaactaaaatatgaacctgaagagacagttgaagtgcctgtactgaaaaagaggcgtgaggtgctccctgtggatataaccaccgctaaagatgcatgtgtcaacaacagtgctctcgggggagaagtttatcgattaccgcctcagaaagaggagacacagtcctgccctaacagtttagaagataacaacttgcaattagaaaaatcagtttctatatacacaccagtagtcagtctctctcctcacaaaaatctgcccgtggatatgcagctgaagaaggaaaagaaatgtgtgaaactcataggagttcccgctgacgctgaggccttaagtgaaagaagtggaaacacccctaactctcccaggttagctgctgaatcaaagcttcaaacagaagttaaagaaggaaaagaaacttcaagcaaattggaaaaagaaacttgtaagaaattacaccctattctatatgtgtcttctaaatctactccagagacccagtgccctcaacagtaaagacttgtctttaataagagtacggtgccacttgcctcaaaagttactatggtgcttaagattgtcttgatctgacatatatcaccttctgggttatttactcattgtgccaggacctggcattttcatgtgcctttgaccaagtgttcagaatttgcttgactctaacctggagagcttcttaagtgatgccccttcatggagcttctatgacagtgaataaactattaattgaaggaaaatgttataattaatgtatctatttgctgcattgtatatggattaaatgataaaaaacaagtaatctaccctcagagccatgtatttgagaatgcttcaatcatattttcctatgtacttttttttataaacttagttttagactatgttgtaaaaatgggaaggttgtaaactatgttgtaaaaataggaaatgtggcttaaaatatatacattatattgtttcaggattttgtcagtgtttaaagaaccaatgttcatctttgtatttatatacatgatttaaattttgtctaaaattttaaataaaactgcagtgatttatccttaaaaaaaaaaaaaaaaaa

SEQ ID NO: 18 - DNA, 2620 bp, Homo sapiens
gcacatgcgccctgacagcccaacaatggcggcgcccgcggagtcgctgaggaggcggaagactgggtactcggatccggagcctgagtcgccgcccgcgccggggcgtggccccgcaggctctccggcccatctccacacgggcaccttctggctgacccggatcgtgctcctgaaggccctagccttcgtgtacttcgtggcattcctggtggctttccatcagaacaagcagctcatcggtgacagggggctgcttccctgcagagtgttcctgaagaacttccagcagtacttccaggacaggacgagctgggaagtcttcagctacatgcccaccatcctctggctgatggactggtcagacatgaactccaacctggacttgctggctcttctcggactgggcatctcgtctttcgtactgatcacgggctgcgccaacatgcttctcatggctgccctgtggggcctctacatgtccctggttaatgtgggccatgtctggtactctttcggatgggagtcccagcttctggagacggggttcctggggatcttcctgtgccctctgtggacgctgtcaaggctgccccagcatacccccacatcccggattgtcctgtggggcttccggtggctgatcttcaggatcatgcttggagcaggcctgatcaagatccggggggaccggtgctggcgagacctcacctgcatggacttccactatgagacccagccgatgcccaatcctgtggcgtactacctgcaccactcaccctggtggttccatcgcttcgagacgctcagcaaccacttcatcgagctcctggtgcccttcttcctcttcctcggccggcgggcgtgcatcatccacggggtgctgcagatcctgttccaggccgtcctcatcgtcagcgggaacctcagcttcctgaactggctgactatggtgcccagcctggcctgctttgatgacgccaccctgggattcttgttcccctctgggccaggcagcctgaaggaccgagttctgcagatgcagagggacatccgaggggcccggcccgagcccagattcggctccgtggtgcggcgtgcagccaacgtctcgctgggcgtcctgctggcctggctcagcgtgcccgtggtcctcaacttgctgagctccaggcaggtcatgaacacccacttcaactctcttcacatcgtcaacacttacggggccttcggaagcatcaccaaggagcgggcggaggtgatcctgcagggcacagccagctccaacgccagcgcccccgatgccatgtgggaggactacgagttcaagtgcaagccaggtgaccccagcagacggccctgcctcatctccccgtaccactaccgcctggactggctgatgtggttcgcggccttccagacctacgagcacaacgactggatcatccacctggctggcaagctcctggccagcgacgccgaggccttgtccctgctggcacacaaccccttcgcgggcaggcccccgcccaggtgggtccgaggagagcactacaggtacaagttcagccgtcctgggggcaggcacgccgccgagggcaagtggtgggtgcggaagaggatcggagcctacttccctccgctcagcctggaggagctgaggccctacttcagggaccgtgggtggcctctgcccgggcccctctagacgtgcaccagaaataaaggcgaagacccagcccctcggcggctcagcaacgtttgcccttccctgcgcccagcccaagctgggcatcgccaagagagacgtggagaggagagcggtgggacccagcccccagcacgggggtccagggtggggtctgttgtcacatactgtggcggctcccaggccctgcccacctggggccccacatccaggccaacccttgtcccaggcgccaggggctctgatctcccatccatcccaccctcctcccagaggcccagcctggggctgtgccgcccacaggagttgagacaatggccatcctgacaccttcctccactacagccctgaccatagacccagccaggtagctcttggggtctctagcgtcccagggcctggtttctgttccctcttcaatggtgtgttcccagccaggtcctgaccctcagagccaagtccctgtcacgtctggggcagccaaaccctcgccccacagggacctggacacgcccggccaggatgtggggttggatgggccattttctgtcctatccctcatctccacccccgccacagcctacacgcatcccacacatgcaggcacacacagcctgtgcacacatgtgttcttggcccggtttcatccccccatgactggtgtctgtgaggtgcagatggacacagcgcacacccagaccctccaccaggctgtgacctcgctgcctctgaggccttgacaaggcccctcaatcggaggacagccggccgtgcacactttcatcatcgtcggacaaacagcgtctactgcacatttttcttattcctattcttgagccatagctatggcatattcttctactattcctattataccacttaccagcttactcgaaaaaaaaaaaaaaaaaa

SEQ ID NO: 19 - DNA, 7254 bp, Homo sapiens
gccgggggcggggggcggcgggcgacggggcgggcgcaggatgagggcggccattgctggggctccgcttcggggaggaggacgctgaggaggcgccgagccgcgcagcgctgcgggggaggcgcccgcgccgacgcggggcccatggccaggaccaccagccagctgtatgacgccgtgcccatccagtccagcgtggtgttatgttcctgcccatccccatcaatggtgaggacccagactgagtccagcacgccccctggcattcctggtggcagcaggcagggccccgccatggacggcactgcagccgagcctcggcccggcgccggctccctgcagcatgcccagcctccgccgcagcctcggaagaagcggcctgaggacttcaagtttgggaaaatccttggggaaggctctttttccacggttgtcctggctcgagaactggcaacctccagagaatatgcgattaaaattctggagaagcgacatatcataaaagagaacaaggtcccctatgtaaccagagagcgggatgtcatgtcgcgcctggatcaccccttctttgttaagctttacttcacatttcaggacgacgagaagctgtatttcggccttagttatgccaaaaatggagaactacttaaatatattcgcaaaatcggttcattcgatgagacctgtacccgattttacacggctgagattgtgtctgctttagagtacttgcacggcaagggcatcattcacagggaccttaaaccggaaaacattttgttaaatgaagatatgcacatccagatcacagattttggaacagcaaaagtcttatccccagagagcaaacaagccagggccaactcattcgtgggaacagcgcagtacgtttctccagagctgctcacggagaagtccgcctgtaagagttcagacctttgggctcttggatgcataatataccagcttgtggcaggactcccaccattccgagctggaaacgagtatcttatatttcagaagatcattaagttggaatatgactttccagaaaaattcttccctaaggcaagagacctcgtggagaaacttttggttttagatgccacaaagcggttaggctgtgaggaaatggaaggatacggacctcttaaagcacacccgttcttcgagtccgtcacgtgggagaacctgcaccagcagacgcctccgaagctcaccgcttacctgccggctatgtcggaagacgacgaggactgctatggcaattatgacaatctcctgagccagtttggctgcatgcaggtgtcttcgtcctcctcctcacactccctgtcagcctccgacacgggcctgccccagaggtcaggcagcaacatagagcagtacattcacgatctggactcgaactcctttgaactggacttacagttttccgaagatgagaagaggttgttgttggagaagcaggctggcggaaacccttggcaccagtttgtagaaaataatttaatactaaagatgggcccagtggataagcggaagggtttatttgcaagacgacgacagctgttgctcacagaaggaccacatttatattatgtggatcctgtcaacaaagttctgaaaggtgaaattccttggtcacaagaacttcgaccagaggccaagaattttaaaactttctttgtccacacgcctaacaggacgtattatctgatggaccccagcgggaacgcacacaagtggtgcaggaagatccaggaggtttggaggcagcgataccagagccacccggacgccgctgtgcagtgacgtggcctgcggccgggctgcccttcgctgccaggacacctgccccagcgcggcttggccgccatccgggacgcttccagaccacctgccagccatcacaaggggaacgcagaggcggaaaccttgcagcatttttatttaaaagaaaagaagaaaaaaaacacccaaccacacaaagaacaaaaccagtaacaaacacaaaggaattcagggtcgctttgcttgctctctgtgctccgtggaggcctccgtgtgccctcgttgccgtggggacccagctccatgcacgtcaacccagtcccgcccagactagtggacagacctggtgtcaccagtttttcctagcatcagtccgaaccatgcgcccgccctgccccaactgtgtgctggtcctgctgtggccgaggggaccgggtgtgtttggctctttatgcccctcccgctgtggtcctggaactcttcaccagggagggagccctgcgggggccgcagctttgtggagggagccgccgtgcttctgtcacctgctccctttcttgcgtctccctgtgatgggcccttaggcctggctgggcccattacatatccctgtggtggctctggtggcagctttctgtggcccctgctgtgttggcaggcaggtttgcgtggtgaggagcgggaggggttggagtggtgcgggagcaggctgccgagtggagggtgccatcgagggctccggatcccttatcctacttagcagtgttggtctctggggctggaagccgagcgcatgctgggagcggtactgtcagaagtgagcccagttagtaccccgctggctcactgcacgagagagtcctgccccgagccctaggtggggccaggaggtgccttggagaagccagccagagcagagagggctgctgacttccgtgtggagcagagaggcctgagggcctcctaaaaggtttaaatgtccacgcctctccagttgctgaagtagggtctgagagaaccctggcatcagcagacccagggtgcttctgtctcctgcagaccacgccagggagtgcagacaccaccgtcacacacgccccttttgtgttttggttcaagtttctcagagcccctcagagcttctacatctgtgcatcagaaatctcacagccttctcatgctgccggctcatctgggcccatagagtgggctttgccagttgctgttgcacaggaggcgagaacagcacacttcaaccccagcttgctggtcggctttcctctagagagagccggttttggggccatttccctttgatgctttggtggccttgccccgctctgcagcacagacaggccagatgcatttgtcctttgcctagctactccccaggtagagagtgctcctggtggcctggcaggtctgggcccttctctccctgcccaggttgtccctggagggcagccctcactccctttgggggagaggcagacattgctgcccacagacctgcctctgactcaactgtgtccaccctccctggtccctacccccaagtcacaggtgactcagcagtgaccctgtgtgccaggccagatccaaactgagagggaaggtgtcgtttttacactgctaatgacgagagtggctctttttagctaggcgagtacagacggggcctgggagggggcagagatgttccccaggccctgcctgtggttcctgcctgggccttggctgctgctgtgtgagagctgcatgtgagcctgtgaccgtgagctggggtgagctgggccgcacctaccctggggccccagggagcaggacgctccggggcccagcacgttgccctgggcctgtggccggagtcggagtcctctctcctcctcctggcttttggaaaggcttggctgtgttggggagtctctcttagccctttcaggaatttctgttcaggcttcctcctcctcatcagctattttacccatctcagaacgtcctgtgtctccatgtaggagagtggctctctcagatctctcagggcgtctggttatagggaaacaagtggagcagggacgtggctttaattggagcactcggctgggctgcttggggagactcttccgtgcgttcttcctctggatagaaccaccacctcctgggcgtcactgacaagctccatcttaacctccaaagccacagaactaggggctcagagccagagctggcagccgccagccaaaatgatgccattgcctgagctgacagccaagcccttctgtgggtcacctttctcctcacccagccccttgctcttcccttttgaaaggcccgtgtgttttctttccttaccctgtgcttgctcatgtctactccggttttctctaccacatccttagagccatcacctggcacgcaggcgccttacattctacggtagaacgtggggtactgtgtgtgcacatagacacacttacgtggaattacagttgtgggtttatccaagatgaggaagatttcacctgctgtttaatagacttggggccatgtgcctccccacacatgggcaaggacaggtggaatgtcgggaccacactgtgcggcttctcggcacaaagcggagggaggctgtggtcgctgccggcctaggtgtcccaggtgccccgcctttctctgggacacagttgggggctggcttctgagggattcctttctcccctctttgtgtggccccagccagggcggtgggcagtcctggtgtagagcacaagcctctccaccctagagaaatgcctctgtaccacggctaccatgtggaaccttaacttgcagaaggcttgttaacaattgttttgagagagatggctggtcatgccacagctgctggggactccgcctactccagccctcttgggacacactgtgggatttgtggcccttccccagaggaattgtggagactgtcccatggaacaaaccctcaggcaccagcacagggctctgggtgactcagtaaaactaacgtttgtctctgacaagatcagctgtaggctcaccggccagagaagaccactgtgagcattttgccgtatatcctgccctgccatttgttcactttttaaactaaaataggaacatccgacacacaccgtttgcatcgtcttctcccttgatattttaagcattttcccatgtcatgagtttctcagaaacatgtttttaacaattgtactatttagtcattgtccatttactataatttatctgaccatttccctactgtaaaatacttaagacggtttctgatttttccactatttaaataatgctgtgatgaatatctttaaaatcttctgatttcttacttttttcccccttagatgcctggaagtggtattttgaggtgaaagagtttgttcattttgaagatatttctgtctctctctcgacctgatgtgtagacgctcacttccagtagcagaaccaccttagttgtgtcttacagattctgaacaaatcggtttctgataagccatgtgttccaaagaatgtctgaataagaccgctctttatttaaatgctaagaggatgtcactactgcaatccatctgtggccgattttttccaagagccaatttccttgttttggttgcaagaacctggctctgcctgcatgtcagctctctgccctccctgctgccgtggctttcaagcgcttggcagaatcttgtacttcgtgtccacaatggtactgaatttgcatctgcacagtcagcagagataacaagtgttgaactgaccttgccacatgcttagtgagtgatttgtaattaagtttatagactcagaaggtatattaggacatttggaatcagtagcagagcaaagcctctttgaaaaaaaccacgtagctgattgggttttacaagagtgcatttgtctcccccttccacccgtggggccccaccttcaggtcttagtggttcacaagagcccagcagccaggctggctttttcattgtagggcgtggttgtcccagctggtgtagatttcaggccgccccccccaactccctgcccacagtgttgcagattgcctggctggcagcaagtccagaccacccaaatttggttggattcttcatttctccactgtagttggggtccattgattgtgcaggggaacgtgcaggaggtttttctaggcaccgtgttcagtgctgcttcactctaccagagattatggccaaattgcacggaatttggtttcttgccctctgaagcctgagggcccccccttgcctggctggttgacagacccggggtggtcactgctgagacttcagagatcgcagctgctgtgagaatacggtgaaggtactttgttctggaagatgttgtcatacacttttccccagttattttcaaacttgacatgagcctatgttgactcactgggtgggggtcccttcttacgcagcacacgtggcaagtgcctgaatcggggctggaggcacttcagagcctctgaggggccaccacttctggcccaaaattgcagggttgtagatgaggctgcctgtggagaactggtgtgaggaggaagctgtttccaacaaagagcactttcatctgttgagatggctgtggtgagcaactgaacgagcctacgtgtgtacctgaattttccccgtaactcatttcttccatatgaagaaacaccaaactatgtacagagaactttttacaaaaggcagaccttttttaagctgtgtaacccacatagcctaaccacctggcagaatgactacgaataggggtcattgtgctggtaaaagcctctattacgactgtaagtaagttggatgttggcaaaattaaattgttacagtatttagagctgctgtagctgttccttcacaacataaaataggataaatgactagtacgtctttcaggtgggtggcaagcagaacatgcgtaatattctctacctggtctgtagctgtaactgtgatgtacagacaaagcaaaaattaaaagaacttatgaaaacaaatgcaatgatactaggatatacacttttgtatttttattcttatataaggttatttgctggctattgttggcctctagttcagtctgtgttatttaaattctaatatatgaattatttgaattgaattcatgttcggggccacgttgttgtatgtattgatgtacagccttgaatgtgaataattattgtaaactatattttacaactttttttctggctttattatataaattttctattgggtcagtgatttaatcatataatttaatgaatctgtttatcctttttttttttccaaatacttgtgctttaggtgtagttaccagatgatgaattttcctcgtatggtcagtagtcttgtaataaaaagcatgtagagtgtaaaaaaaaaaaaaaaaaaa

SEQ ID NO: 20 - DNA, 3053 bp, Homo sapiens
cattccatggtcccgcagccccaggcccacactgaaagcatgtcgatccaggagaacatatcatccctgcagcttcggtcatgggtctctaagtcccaaagagacttagcaaagtccatcctgattggggctccaggagggccagcggggtatctgcggcgggccagtgtggcccaactgacccaggagctgggcactgccttcttccagcagcagcagctgccagctgctatggcagacaccttcctggaacacctctgcctactggacattgactccgagcccgtggctgctcgcagtaccagcatcattgccaccatcgggccagcatctcgctccgtggagcgcctcaaggagatgatcaaggccgggatgaacattgcgcgactcaacttctcccacggctcccacgagtaccatgctgagtccatcgccaacgtccgggaggcggtggagagctttgcaggttccccactcagctaccggcccgtggccatcgccctggacaccaagggaccggagatccgcactgggatcctgcaggggggtccagagtcggaagtggagctggtgaagggctcccaggtgctggtgactgtggaccccgcgttccggacgcgggggaacgcgaacaccgtgtgggtggactaccccaatattgtccgggtcgtgccggtggggggccgcatctacattgacgacgggctcatctccctagtggtccagaaaatcggcccagagggactggtgacccaagtggagaacggcggcgtcctgggcagccggaagggcgtgaacttgccaggggcccaggtggacttgcccgggctgtccgagcaggacgtccgagacctgcgcttcggggtggagcatggggtggacatcgtctttgcctcctttgtgcggaaagccagcgacgtggctgccgtcagggctgctctgggtccggaaggacacggcatcaagatcatcagcaaaattgagaaccacgaaggcgtgaagaggtttgatgaaatcctggaggtgagcgacggcatcatggtggcacggggggacctaggcatcgagatcccagcagagaaggttttcctggctcagaagatgatgattgggcgctgcaacttggcgggcaagcctgttgtctgtgccacacagatgctggagagcatgattaccaagccccggccaacgagggcagagacaagcgatgtcgccaatgctgtgctggatggggctgactgcatcatgctgtcaggggagactgccaagggcaacttccctgtggaagcggtgaagatgcagcatgcgattgcccgggaggcagaggccgcagtgtaccaccggcagctgtttgaggagctacgtcgggcagcgccactaagccgtgatcccactgaggtcaccgccattggtgctgtggaggctgccttcaagtgctgtgctgctgccatcattgtgctgaccacaactggccgctcagcccagcttctgtctcggtaccgacctcgggcagcagtcattgctgtcacccgctctgcccaggctgcccgccaggtccacttatgccgaggagtcttccccttgctttaccgtgaacctccagaagccatctgggcagatgatgtagatcgccgggtgcaatttggcattgaaagtggaaagctccgtggcttcctccgtgttggagacctggtgattgtggtgacaggctggcgacctggctccggctacaccaacatcatgcgggtgctaagcatatcctgagacgcccctccctcctctggcccagcctacccttgtaccccatcccttcctccccagtctacgttctccagcccacacccctccaaagccccacctttaagtcctctcttctctattcctgaccctccctacctgaggcctatctgagactataactgtcatctagccccttcgaggttgccccttccccatctccatttcacacaggtcctgaaagtctgtgtccaattatgcactggccacccaacagcaccaattgtacattccctgcatccaatctgctcagcaggccctaagatgccttgagtctttaatcccagtttggctggttaattccataaccccaggcatcccatcccttggggtgggggagaggggagacagggcaatcttgtccacagtctcccattctcatatgtagccctcatgataatctgggcatctcgtgccagggcaggctaccccttcatggtgactaacagttacatgaaagtccacgcttttgggaaaactgggtgggatggatgctggggagaagtgagggctgggcagctgattttgtcactgtcttcacaacctccgtgctgggcttgtagaccactgtcctggctgctctcatgcctgcctgataccctgcttggtcaaatccccggctgcttccttctgcacccagaaattccttcccactcatgttgttcccacacacaaaccaagagccaaaaatgagtgtgtctattttatatttaaacccagctgtttggaagcaattataaaacttctcccacacaccaagaaccccagaactccccacccagaaggaaaggagacttagggtcctggtcccacagtctttaatgctgtagagtaggagaggggacaggcagagtgagggtggtaaaggactactcttgtccctgagaaggcagaggtgctggctggcctgcccttccccaggcttggataccttgggcctttcccttattcctgcaccaacagcaaactcagaaggaaaaaacaaaacaaaactctaaaggtaaacgcggtccttctccctctctcatgtggctcctcctgcccctacctgggagaaggttcaagtattgcgctgatgggtttgggcaaggaccacagaggctggcatggagaggccctgcctcctcctactcctcctgtctccttacctgaaaaggaggggagggatggataaaggaatgggtaaacaaaattaattggtaacacataaagttagtaaatgaataaatacaattaaataaatgaaaaaaaaaaaa

SEQ ID NO: 21 - DNA, 2477 bp, Homo sapiens
ggcgctctgggcccgtagcgctccgcgggaaggaggctggatgcccggcagcagtggggcggggatggaggcggccgtggcgccggggagggatgcgccggcacccgcggcgagtcagcccagcggctgcgggaaacacaactcgccggagaggaaagtttatatggactataatgcaacgactcccctggagccagaagttatccaggccatgaccaaggccatgtgggaagcctggggaaatcccagcagcccgtattcagcaggaagaaaggccaaggatattataaatgcagctcgggaaagcctcgcgaagatgataggggggaaacctcaagatataatcttcacttccgggggcactgagtcaaataatttagtaatccattctgtggtgaaacatttccacgcaaaccagacctcaaagggacacacaggtgggcaccacagcccagtgaagggggccaagccccatttcattacttcctcggtggaacacgactccatccggctgcccctggagcacctggtggaagaacaagtggcagcggtcacctttgtcccggtgtccaaggtgagcgggcaggcagaggtggacgacatcctcgcggcagtccgcccgaccacacgcctcgtgaccatcatgctggccaacaatgagactggcattgtcatgcctgtccctgaaatcagtcagcgcattaaagccctgaaccaggaacgggtggcagctgggctacctcccatcctcgtgcacacggatgctgcacaggccttggggaagcagcgcgtggatgtggaggacctgggcgtggacttccttacaatcgtggggcacaagttttatggtcccaggattggcgcactttatatacgaggacttggtgaatttacccctctctaccctatgctatttggaggtggacaagaacggaatttcaggccagggacagagaacaccccaatgattgctggccttgggaaggccgcggagctggtgacccagaactgcgaggcttatgaggcccacatgagggacgtccgcgactacctggaagagaggctggaagctgaattcggtcagaagagaatccatctgaatagccagtttccaggcacccagcggcttcccaatacctgtaacttttccatccggggaccccggcttcaaggccacgtggtgcttgcgcagtgccgagtgctgatggccagtgtgggggccgcgtgccactcggaccacggggaccagccgtccccagtgctgctgagctacggtgtccccttcgacgtggccaggaacgcgctccggctcagcgtgggccgcagcaccaccagggccgaggtggacctcgtcgtgcaggacctgaagcaggccgtggcgcagctggaggaccaggcctagcactggggccgccttccccaccccgcttctgggaagcccgtggcagggcacagggttgtccctccagttccctcctgagggctgtgccaggatgactgtctcatgccccctctgcattttgtcctggagtgccagcgagtgtgcacccccagtttccttccctggacccctgcagagctcacagggcccaggacaccaacgccgcataggactgcccacatgggaccgcccacataggaccgcccacataggaccgcccacatgggaccgcccacatgggaccgcccacatgggaccgcccacatagaaccgtcctccagtggtgaagcggaaacacttagctttatccaccctccccactgggaactgggcacgcctgttgtgagtgccctttcctggaaggtgtttttatctggaagatagaatccaagtatttataactcattgtcagccaaatgctatcatgaacgtaggaaacttgatttttttgttttgatcatggcctctacatgcacctttccagagtggtctcttctcaggccctttttagtccctttccaaagctgcccccaccaccctgctcctctggcctcagtgcacagtggcccccagcctcggccaggcctgctctgctcagcgcccaccgcccaccacccctcctgcttccccgagcctgaccctgttccgcccactggcaatcagggctgcgcacttccctgtccacggtccccaggccttcctgtcttgtcccttttgatcattattaactcagggtttcagctccaacctcgctgagttggtgcagctccaggtcattcctggggtgggaatcggatcatccctgactcagcttttaccttaattttatttgcagaggattcttttctcaaaatgctctggcatttggacacacatcacatgtcgatatttgcataggagtcattttcagtggaataacatttttaatgtgtggttttacggttcaaggaactacttgatgattttgaggaaacacttgccagaaactaaattaacgaataaaagatttcagtgcccgaaaaaaaaaaaaaaaaaa

SEQ ID NO: 22 - DNA, 2473 bp, Homo sapiens
atcagaggaagaggaaggggcggagctgctttgcggccggccgcggagcagtcagccgactacagagaagggtaatcgggtgtccccggcgccgcccggggccctgagggctggctagggtccaggccgggggggacgggacagacgaaccagccccgtgtaggaagcgcgacaatgccccgctacggagcgtcactccgccagagctgccccaggtccggccgggagcagggacaagacgggaccgccggagcccccggactcctttggatgggcctggtgctggcgctggcgctggcgctggcgctggcgctggctctgtctgactctcgggttctctgggctccggcagaggctcaccctctttctccccaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccccccagccccaggtgcccctgtcagccgcatcctcttcctcactgacctgcactgggatcatgactacctggagggcacggaccctgactgtgcagacccactgtgctgccgccggggttctggcctgccgcccgcatcccggccaggtgccggatactggggcgaatacagcaagtgtgacctgcccctgaggaccctggagagcctgttgagtgggctgggcccagccggcccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgccgtctggctactctttgtgcccagctctctgcccgtgctgacagccctgctctgtgccgccacctgatgccagatgggagcctcccagaggcccagagcctgtggccaaggccactgttttgctagggccccagggcccacatttgggaaagttcttgatgtaggaaagggtgaaaaagcccaaatgctgctgtggttcaaccaggcaagatcatccggtgaaagaaccagtccctgggccccaaggatgccggggaaacaggaccttctcctttcctggagctggtttagctggatatgggagggggtttggctgcctgtgcccaggagctagactgccttgaggctgctgtcctttcacagccatggagtagaggcctaagttgacactgccctgggcagacaagacaggagctgtcgccccaggcctgtgctgcccagccaggaaccctgtactgctgctgcgacctgatgctgccagtctgttaaaataaagataagagacttggactccaaaaaaaaaaaaaaa

SEQ ID NO: 23 - DNA, 4624 bp, Homo sapiens
tcggcgctcgcgggctcggcgggctgtgcgcgcccactccggctccagcggccagcgcgcgcgggcccaggccgcccggctccagcccagcagtagcggcagcagcggcggcggcggcagtgcgcgcgaggccctgcgcccccagcagctcctccctggcgccgtgcatggagacgcggcccgccacccgccgctgagcccccgccgcccggccgggacccgccagggctggggtggcctcgggctccggccggccccgccgcccgagggctgcgcgcggcccgcgggcctcgccgccccgcgcggatcgtcgcggcccggccgtcccgtcccaggaagtggccgtcctgagcgccatggctcactccccggtgcagtcgggcctgcccggcatgcagaacctaaaggcagacccagaagagctttttacaaaactagagaaaattgggaagggctcctttggagaggtgttcaaaggcattgacaatcggactcagaaagtggttgccataaagatcattgatctggaagaagctgaagatgagatagaggacattcaacaagaaatcacagtgctgagtcagtgtgacagtccatatgtaaccaaatattatggatcctatctgaaggatacaaaattatggataataatggaatatcttggtggaggctccgcactagatctattagaacctggcccattagatgaaacccagatcgctactatattaagagaaatactgaaaggactcgattatctccattcggagaagaaaatccacagagacattaaagcggccaacgtcctgctgtctgagcatggcgaggtgaagctggcggactttggcgtggctggccagctgacagacacccagatcaaaaggaacaccttcgtgggcaccccattctggatggcacccgaggtcatcaaacagtcggcctatgactcgaaggcagacatctggtccctgggcataacagctattgaacttgcaagaggggaaccacctcattccgagctgcaccccatgaaagttttattcctcattccaaagaacaacccaccgacgttggaaggaaactacagtaaacccctcaaggagtttgtggaggcctgtttgaataaggagccgagctttagacccactgctaaggagttattgaagcacaagtttatactacgcaatgcaaagaaaacttcctacttgaccgagctcatcgacaggtacaagagatggaaggccgagcagagccatgacgactcgagctccgaggattccgacgcggaaacagatggccaagcctcggggggcagtgattctggggactggatcttcacaatccgagaaaaagatcccaagaatctcgagaatggagctcttcagccatcggacttggacagaaataagatgaaagacatcccaaagaggcctttctctcagtgtttatctacaattatttctcctctgtttgcagagttgaaggagaagagccaggcgtgcggagggaacttggggtccattgaagagctgcgaggggccatctacctagcggaggaggcgtgccctggcatctccgacaccatggtggcccagctcgtgcagcggctccagagatactctctaagtggtggaggaacttcatcccactgaaattcctttggcatttggggttttgtttttccttttttccttcttcatcctcctccttttttaaaagtcaacgagagccttcgctgactccaccgaagaggtgcgccactgggagccaccccagcgccaggcgcccgtccagggacacacacagtcttcactgtgctgcagccagatgaagtctctcagatgggtggggagggtcagctccttccagcgatcattttattttattttattacttttgtttttaattttaaccatagtgcacatattccaggaaagtgtctttaaaaacaaaaacaaaccctgaaatgtatatttgggattatgataaggcaactaaagacatgaaacctcaggtatcctgctttaagttgataactccctctgggagctggagaatcgctctggtggatgggtgtacagatttgtatataatgtcatttttacggaaaccctttcggcgtgcataaggaatcactgtgtacaaactggccaagtgcttctgtagataacgtcagtggagtaaatattcgacaggccataacttgagtctattgccttgcctttattacatgtacattttgaattctgtgaccagtgatttgggttttattttgtatttgcagggtttgtcattaataattaatgcccctctcttacagaacactcctatttgtacctcaacaaatgcaaattttccccgtttgccctacgccccttttggtacacctagaggttgatttcctttttcatcgatggtactatttcttagtgttttaaattggaacatatcttgcctcatgaagctttaaattataattttcagtttctccccatgaagcgctctcgtctgacatttgtttggaatcgtgccactgctggtctgcgccagatgtaccgtcctttccaatacgattttctgttgcaccttgtagtggattctgcatatcatctttcccacctaaaaatgtctgaatgcttacacaaataaattttataacacgcttattttgcatactccttgaaatgtgactcttcagaggacagggcacctgctgtgtatgtgtggccgtgcgtgtgtactcgtggctgtgtgtgtgtgatgagacactttggaagactccagggagaagtccccaggcctggagctgccgagtgcccaggtcagcgccctggactgcttgcgcacttgctcaccgagatgatgcagttggaggttgctgatctgtgcgattgctgtagcggttgccggggaccttaagagttattttgcttctctggaaggggcctatgcttgctaggcaggcagccagtgtgtctgtttttcttggtttgctgtgggaccttgcttggcgagggggaaaatctctgggtttctggagtgggagggttcgtgcagcagctgttgactggtacatgaagcattcttttatgtttgttgaagctgatgattgacatctcccgtgggtgtgccagttcttgtggagttaagacaggatttttggaagcaaggaagttagtgggtgagcttggggatgtagctcagctatctgctggtctagtggcctctaagctatagggaggggacagagccctgagctacagatgcttgagtgggttattgtgtcggtttgctagtgcagtctggtttttaagctctaaaattgaggtattttattagaagtggatttgggttgaactcttaatttgtataagggatatattttggttggggaaatagaactgagttgctaattcttattgtactcattactccatacaagaatgttatgttgaataataaaattggagaagatttcattttgtgtttccagggagtattctgtgtggggaactgtttccttacgtgaggccggcggcataagtcaaagatgagttttgtccttgcgaatcacacagattgagtctgtgttccccagggtgtgccgttacctgatttttaagtgagccagggcggacagcagcttttctgatttacagagttcttcagatttacaaatggacaatgacatcacagtttttagcactgaagccagtctcatgctagtaacagtgggtgagccgctcgagggactgggttctaatgaatactggtatgaacggggagtctctgcagtcgccagacaaatcatactcagccccttcccccgtagagcaacaagtggttcttttagagttgactggcagcatttcctgtcgggggaggtggggtttgatggagttagaaagctcgcctctgtgtacattctctcctgggctgttactttctgtagacgcacaaaatcagccccaatgtttttaagggcatcttagccaaggaagctggcttttgtgtcgccacttccaggcctgcattaagagagagcccaggcaccagggctaccactggaacctgcctcagcgtcaactgctgctggtctgtagccaggcccagcctttgagacgggtttactgtcaccagtagcctctcagtgccagccctgagctgctcctggctcagctgcccagagcctgcagcctggggaggtactcagcctctgggagacgagggccgtggactgggtggctggtagctcctgcgtttttgagctgtgtcctggctggctgctgccaatgaggtggacaccagtgtggtttggggtgcactggccacttcttgctgggttctgattttcttggaagtgcatctgccttccttatccaatagttttatccctgcattgctcttgtgaagtggctggtttggttctgtatgtagcattttgtacctttcctctggcaaaacactgtcagtttataaacattttttatatttccctcctttaaaaacagcttgtgtatttctgctataaaatgtgtcagcaaaggcagagtgacctaatagggcatgttcttaagcacagggactgtatcatgcaggggccaataaagctcaagaaaacgag

SEQ ID NO: 24 - DNA, 4830 bp, Homo sapiens
caaccggttccgagtttgaggcactaggaggagggggagaagcggctgcagcggccgcggcaggagcagcgggagctacagcatcagcaagagcaacagtagctacagccccggcggcggtgcctgttccagtctttgctgctgcagtccgtgcaaccacccagagggggaggggggaaccaccagtcgctgaggaacaagagaaggggggaaagtttaggcgagccttgggggggggggggccagcgccggagccgcgtgagagagggagccgtgttttggtaggggggagtcggactgcaactggcagcagagcgtctccccggccgtgtggactctacaccccctactcctgccgcttctgctgctgcctgtggctggagggtccccctggggctgaatctttgggacttgaccccgttccctcccccttccctcactccccagccgggcgggagcatttattccccagattaattccccttttgggggggggcgggtgtgtgtgtgtgtgtgtgtgtgtgtgtgtgttgggggaagcgtccctgaaatagtaaatattattgagctctttttgcccttttcctgtccgtttttttaatttccttttttgaggtgggaaaactgaaacccaccttgattcgtcccctctcccccctccccaccttccctcgccctaatcccccaacgaggaaggaaggagcagttggttcaatctctggtaatctatgccagcaattatgacaatgttagcagaccatgcagctcgtcagctgcttgatttcagccaaaaactggatatcaacttattagataatgtggtgaattgcttataccatggagaaggagcccagcaaagaatggctcaagaagtactgacacatttaaaggagcatcctgatgcttggacaagagtcgacacaattttggaattttctcagaatatgaatacgaaatactatggactacaaattttggaaaatgtgataaaaacaaggtggaagattcttccaaggaaccagtgcgaaggaataaaaaaatacgttgttggcctcattatcaagacgtcatctgacccaacttgtgtagagaaagaaaaggtgtatatcggaaaattaaatatgatccttgttcagatactgaaacaagaatggcccaaacattggccaacttttatcagtgatattgttggagcaagtaggaccagcgaaagtctctgtcaaaataatatggtgattcttaaactcttgagtgaagaagtatttgatttctctagtggacagataacccaagtcaaatctaagcatttaaaagacagcatgtgcaatgaattctcacagatatttcaactgtgtcagtttgtaatggaaaattctcaaaatgctccacttgtacatgcaaccttggaaacattgctcagatttctgaactggattcccctgggatatatttttgagaccaaattaatcagcacattgatttataagttcctgaatgttccaatgtttcgaaatgtctctctgaagtgcctcactgagattgctggtgtgagtgtaagccaatatgaagaacaatttgtaacactatttactctgacaatgatgcaactaaagcagatgcttcctttaaataccaatattcgacttgcgtactcaaatggaaaagatgatgaacagaacttcattcaaaatctcagtttgtttctctgcacctttcttaaggaacatgatcaacttatagaaaaaagattaaatctcagggaaactcttatggaggcccttcattatatgttgttggtatctgaagtagaagaaactgaaatctttaaaatttgtcttgaatactggaatcatttggctgctgaactctatagagagagtccattctctacatctgcctctccgttgctttctggaagtcaacattttgatgttcctcccaggagacagctatatttgcccatgttattcaaggtccgtttattaatggttagtcgaatggctaaaccagaggaagtattggttgtagagaatgatcaaggagaagttgtgagagaattcatgaaggatacagattccataaatttgtataagaatatgagggaaacattggtttatcttactcatctggattatgtagatacagaaagaataatgacagagaagcttcacaatcaagtgaatggtacagagtggtcatggaaaaatttgaatacattgtgttgggcaataggctccattagtggagcaatgcatgaagaggacgaaaaacgatttcttgttactgttataaaggatctattaggattatgtgaacagaaaagaggcaaagataataaagctattattgcatcaaatatcatgtacatagtaggtcaatacccacgttttttgagagctcactggaaatttctgaagactgtagttaacaagctgttcgaattcatgcatgagacccatgatggagtccaggatatggcttgtgatactttcattaaaatagcccaaaaatgccgcaggcatttcgttcaggttcaggttggagaagtgatgccatttattgatgaaattttgaacaacattaacactattatttgtgatcttcagcctcaacaggttcatacgttttatgaagctgtggggtacatgattggtgcacaaacagatcaaacagtacaagaacacttgatagaaaagtacatgttactccctaatcaagtgtgggatagtataatccagcaggcaaccaaaaatgtggatatactgaaagatcctgaaacagtcaagcagcttggtagcattttgaaaacaaatgtgagagcctgcaaagctgttggacacccctttgtaattcagcttggaagaatttatttagatatgcttaatgtatacaagtgcctcagtgaaaatatttctgcagctatccaagctaatggtgaaatggttacaaagcaaccattgattagaagtatgcgaactgtaaaaagggaaactttaaagttaatatctggttgggtgagccgatccaatgatccacagatggtcgctgaaaattttgttccccctctgttggatgcagttctcattgattatcagagaaatgtcccagctgctagagaaccagaagtgcttagtactatggccataattgtcaacaagttagggggacatataacagctgaaatacctcaaatatttgatgctgtttttgaatgcacattgaatatgataaataaggactttgaagaatatcctgaacatagaacgaactttttcttactacttcaggctgtcaattctcattgtttcccagcattccttgctattccacctacacagtttaaacttgttttggattccatcatttgggctttcaaacatactatgaggaatgtcgcagatacgggcttacagatactttttacactcttacaaaatgttgcacaagaagaagctgcagctcagagtttttatcaaacttatttttgtgatattctccagcatatcttttctgttgtgacagacacttcacatactgctggtttaacaatgcatgcatcaattcttgcatatatgtttaatttggttgaagaaggaaaaataagtacatcattaaatcctggaaatccagttaacaaccaaatctttcttcaggaatatgtggctaatctccttaagtcggccttccctcacctacaagatgctcaagtaaagctctttgtgacagggcttttcagcttaaatcaagatattcctgctttcaaggaacatttaagagatttcctagttcaaataaaggaatttgcaggtgaagacacttctgatttgtttttggaagagagagaaatagccctacggcaggctgatgaagagaaacataaacgtcaaatgtctgtccctggcatctttaatccacatgagattccagaagaaatgtgtgattaaaatccaaattcatgctgttttttttctctgcaactcgttagcagaggaaaacagcatgtgggtatttgtcgaccaaaatgatgccaatttgtaaattaaaatgtcacctagtggccctttttcttatgtgtttttttgtataagaaattttctgtgaaatatccttccattgtttaagcttttgttttggtcatctttatttagtttgcatgaagttgaaaattaaggcatttttaaaaattttacttcatgcccatttttgtggctgggctggggggaggaggcaaattcgatttgaacatatacttgtaattctaatgcaaaattatacaatttttcctgtaaacaataccaatttttaattagggagcattttccttctagtctatttcagcctagaagaaaagataatgagtaaaacaaattgcgttgtttaaaggattatagtgctgcattgtctgaagttagcacctcttggactgaatcgtttgtctagactacatgtattacaaagtctctttggcaagattgcagcaagatcatgtgcatatcatcccattgtaaagcgacttcaaaaatatgggaacacagttagttatttttacacagttctttttgtttttgtgtgtgtgtgctgtcgcttgtcgacaacagctttttgttttcctcaatgaggagtgttgctcatttgtgagccttcattaactcgaagtgaaatggttaaaaatatttatcctgttagaataggctgcatctttttaacaactcattaaaaaacaaaacaactctggcttttgagatgacttatactaatttacattgtttaccaagctgtagtgctttaagaacactacttaaaaagcaaaataaacttggtttacattta

SEQ ID NO: 25 - DNA, 3867 bp, Homo sapiens
actgtcttcctcattggcgccgtgcagagaggcggaatgttcaactcctaactgcggcggaaacgtgggagccgcgcgggccgctgtcgtcccaacccccgccgccctcgtcgcgcgcggggcctccgcgcccccggctgctgctcacgccccgcccgggagccagattttgtggaagtataatactttgtcattatgagatgtcgtctctcggtgcctcctttgtgcaaattaaatttgatgacttgcagttttttgaaaactgcggtggaggaagttttgggagtgtttatcgagccaaatggatatcacaggacaaggaggtggctgtaaagaagctcctcaaaatagagaaagaggcagaaatactcagtgtcctcagtcacagaaacatcatccagttttatggagtaattcttgaacctcccaactatggcattgtcacagaatatgcttctctgggatcactctatgattacattaacagtaacagaagtgaggagatggatatggatcacattatgacctgggccactgatgtagccaaaggaatgcattatttacatatggaggctcctgtcaaggtgattcacagagacctcaagtcaagaaacgttgttatagctgctgatggagtattgaagatctgtgactttggtgcctctcggttccataaccatacaacacacatgtccttggttggaactttcccatggatggctccagaagttatccagagtctccctgtgtcagaaacttgtgacacatattcctatggtgtggttctctgggagatgctaacaagggaggtcccctttaaaggtttggaaggattacaagtagcttggcttgtagtggaaaaaaacgagagattaaccattccaagcagttgccccagaagttttgctgaactgttacatcagtgttgggaagctgatgccaagaaacggccatcattcaagcaaatcatttcaatcctggagtccatgtcaaatgacacgagccttcctgacaagtgtaactcattcctacacaacaaggcggagtggaggtgcgaaattgaggcaactcttgagaggctaaagaaactagagcgtgatctcagctttaaggagcaggagcttaaagaacgagaaagacgtttaaagatgtgggagcaaaagctgacagagcagtccaacaccccgctgctgccttcctttgagattggtgcatggacggaagacgatgtgtattgttgggttcagcagctcgtcagaaaaggtgactcttcagcagagatgagtgtatatgcaagcttgtttaaagaaaacaacattacagggaagcggctgctgctgctggaggaagaagacctgaaagacatgggcattgtctccaaggggcatatcattcacttcaagtcagccattgagaaattaacccatgattacataaatttgtttcacttcccaccactaattaaggactcaggaggtgaacctgaagaaaatgaggaaaaaatagtgaacctggaactggtttttggttttcacttgaaaccaggaactggcccacaggattgtaagtggaaaatgtatatggagatggatggggatgaaattgcaataacctacataaaagatgtgacattcaacactaacctacctgatgcggagattttaaagatgacaaagccaccatttgtaatggagaagtggattgtaggaatagcaaaaagtcagactgtggagtgcactgtcacatatgagagtgatgttagaactccaaaaagcactaaacatgtccattcgattcagtggagtagaacaaaacctcaggatgaagtgaaagcagtccaacttgccattcagacattattcaccaattcagatggcaaccctggaagcaggtccgactcaagtgctgattgccagtggttagatactctgaggatgcggcagattgcatccaacacttctttacagcgttcccagagcaatcctattctggggtcaccgttcttctcacactttgatggccaggattcctacgctgctgctgtgagacggccccaggtgcccattaagtatcaacagattacacctgtgaaccagtccagaagctcgtctcctactcagtatggactgaccaaaaacttctcttccctacatctcaactctagggacagtggcttttccagtggcaatactgacacctcttcagagaggggtcgatactcagacagaagcaggaacaaatatggacgtggtagtatatcactcaattcttctcctagaggaagatacagtggaaagagtcagcattccactccttcaagaggaagataccctggaaagttctacagggtttctcagtcagcactcaatcctcaccagtcgcctgacttcaagagaagccccagggacctccaccaacccaacaccataccagggatgcctttgcaccctgagactgactcaagagccagtgaagaggacagcaaagtcagcgaagggggctggacaaaagtggaataccggaaaaagccccacaggccatctcccgccaaaaccaataaagagagagccagaggggaccaccgtggatggagaaacttttgatgaattgaactacatagcttttctaagcaggttaaaaaaaaaaaaaaaaagaaatgtaatggtttttgataatatgatcccttcagattgaattaacgaaaagacaacacttccagtttttggattgggaaataccttctaattgagactatagccaaaccagggccaaaattatggatattggtcacccagtgatcataactaggcttgaaaatcactacacatattttctgccttgagtgaacatttttagaggaaaggttatgccatctttttaccctaaccactgatattctggttagcagggccaggacaaggggaaggaaaatgaggtcaacaaaaaaatcaaatttttaggaaaagataagatgaatgttactgatttttccttttggctgaggctgcaatatggcctggcaaggcactgttactgatcttgtctttaacattttgatattttgttcatcataatttttgcatttatttttttaaatattgcattaaaatatcatttagcttgattatcgagttttttggtttgaggttttttgttgcttcttttttcttttctttctttccccctcttttttttggatgtccccttaaattttgtgcccaaggcaggtacctcactcatctcatccttggctcagccctgctggttagtatttagtatttattttagtaagatatttgtgtctgtatgatggtcagagttgaactgatctggcttgtcatttttcagtaataaaaaaagttactgaatttaatgttgaatatgatgcatatctcattcattacgatttatcagaaaccaaagatttaaattgcctagatttgtggttctttctcttcctaagttcccagcgactgctttcaaatactattttctaaatttcaccaaaggagcaaagaggataaaacaacactccataaaggcctcttgggatgtcagaaatctaaaatctaaaagaaaacagacacagagcaagacaataacatcacaagctaaaagccagagaaatttaaaattaccaacatccttgttggagtaagacagtaaatatcagccttgcagcaagacagctctgagcagctgtgggcaaagaggtaaaccagtgggggtgcaaggagactgtctgcagcttagggcagaaatggtgggatccaacttgtgaaatgcttcatgttttacaaaccaaaaagtcaggtagcaacaaacttattgtatgtcaaatcaataaatgttactttcaataaaaaa

(19) United States
(12) Patent Application Publication
(10) Pub. No.: US 2011/0256545 A1
(43) Pub. Date: Oct. 20, 2011

(54) mRNA expression-based prognostic gene signature for non-small cell lung cancer

(76) Inventors: Nancy Lan Guo, Morgantown, WV (US); Ying-Wooi Wan, Morgantown, WV (US)

(21) Appl. No.: 13/065,705

(22) Filed: Mar. 28, 2011

Related U.S. Application Data
(60) Provisional application No. 61/342,458, filed on Apr. 14, 2010.

(51) Int. Cl. C12Q 1/68; C07H 21/02

(52) U.S. Cl. 435/6.13; 536/23.1

(57) ABSTRACT

A non-small cell lung cancer postoperative survival prognosticator comprising a detection mechanism consisting of 15-gene, 12-gene, and 16-gene signature and methods of use. Also provided are the identification of various subsets from the 25 prognostic signature genes with potential of operative survival prognosticator for non-small cell lung cancer patients in all tumor stage and early stage and potential for chemoresponse with a method of use.

### CROSS-REFERENCE TO RELATED APPLICATIONS

This application claims priority from provisional application No. 61/342,458 and filed on Apr. 14, 2010.

### STATEMENT REGARDING FEDERALLY SPONSORED RESEARCH OR DEVELOPMENT

This invention was made with government support under Grant No. R01LM009500 awarded by the NIH. The United States government has certain rights in the invention.

### REFERENCE TO SEQUENCE LISTING, A TABLE, OR A COMPUTER PROGRAM LISTING COMPACT DISC APPENDIX

This application contains a Sequence Listing submitted on compact disk containing file name Seq. 482. The sequence listing on the compact disc is incorporated by reference herein in its entirety.

### BRIEF DESCRIPTION OF THE SEVERAL VIEWS OF THE DRAWINGS

The following figures are not drawn to scale and are for illustrative purposes only.

FIG. 1 is a Kaplan-Meier analysis of the 15-gene prognostic classifier on overall survival prediction.

FIG. 2 is a Kaplan-Meier analysis of the 16-gene prognostic classifier on overall survival prediction.

FIG. 3 is a Kaplan-Meier analysis of the 12-gene prognostic classifier on overall survival prediction.

FIG. 4 is a Kaplan-Meier analysis of the 15-gene prognostic model in early stages patients.

FIG. 5 is a Kaplan-Meier analysis of the 12-gene prognostic model in early stages patients.

FIG. 6 is a Kaplan-Meier analysis of the 16-gene prognostic model in early stages patients.

FIG. 7 is the comparison of prognostic performance of the 15-gene, 12-gene, and 16-gene prognostic models and molecular prognostic models.

FIG. 8 is a Gene Set Enrichment Analysis (GSEA) of the 15-gene and 12-gene along with 14 published gene signatures (listed in Table 5) in lung cancer.

FIG. 9 is the functional pathway analysis of the 12-gene signature using Ingenuity Pathway Analysis (IPA) core analysis.

FIG. 10 is the functional pathway analysis of the 15-gene signature using Ingenuity Pathway Analysis (IPA) core analysis.

FIG. 11 is the curated interactions among the 25 signature genes and 10 prominent lung cancer hallmarks using Pathway Studio.

### DETAILED DESCRIPTION OF THE INVENTION

A first embodiment can be an expression profile-defined prognostic model able to predict an individual patient's risk for recurrence across independent cohorts with non-small cell lung cancer. Additionally, the expression profile-defined prognostic model may be used to place a patient into one of two groups in order to properly treat and manage a patient. The expression based profile-defined prognostic model has been developed and is a highly accurate predictor of overall survival in individual patients. The expression based profile-defined prognostic model can be a gene signature such as the 15-, 12-, and 16-gene signatures comprised of the genes in Table 1, Table 2, and Table 3, respectively.

TABLE 1
The identified 15 prognostic signature genes for non-small cell lung cancer

| Probe Set | Name | Gene Symbol | Function | Sequence ID |
|---|---|---|---|---|
| 208772_at | SeqID No1 | ANKHD1 | Unknown | NM_017747 |
| 206150_at | SeqID No2 | CD27 | B-cell activation and | NM_001242 |
| | | | immunoglobulin synthesis; | |
| | | | signaling transduction | |
| 214717_at | SeqID No3 | DKFZp434H1419 | Unknown | |
| 210762_s_at | SeqID No4 | DLC1 | A candidate tumor suppressor | NM_182643.2 |
| | | | gene | |
| 213779_at | SeqID No5 | EMID1 | Unknown | NM_133455 |
| 211603_s_at | Seq ID No6 | ETV4 | Cellular movement | NM_001079675 |
| 205308_at | Seq ID No7 | FAM164A | Unknown | NM_016010 |
| 211327_x_at | Seq ID No8 | HFE | Iron absorption | NM_000410 |
| 204854_at | Seq ID No9 | LEPREL2 | Collagen biosynthesis, folding, | NM_014262 |
| | | (GPR162) | and assembly | |
| 205171_at | Seq ID No10 | PTPN4 | Cell growth, differentiation, | NM_002830 |
| | | | mitotic cycle, and oncogenic | |
| | | | transformation | |
| 201107_s_at | Seq ID No11 | THBS1 | Cell-to-cell and cell-to-matrix | NM_003246 |
| | | | interactions. | |
| 215598_at | Seq ID No12 | TTC12 | Binding | NM_017868 |
| 201581_at | Seq ID No13 | TXNDC13 | Cell redox homeostasis, electron | NM_021156 |
| | | (TMX4) | transport chain | |
| 218340_s_at | Seq ID No14 | UBA6 | Ubiquitin-activating protein | NM_018227 |
| 207296_at | Seq ID No15 | ZNF343 | Unknown | NM_024325 |

TABLE 2
The identified 12 prognostic signature genes for non-small cell lung cancer

| | | Gene | | |
|---|---|---|---|---|
| Probe Set | Name | Symbol | Function | Sequence ID |
| 212041_at | Seq ID No16 | ATP6V0D1 | Atpase | NM_004691 |
| 221685_s_at | Seq ID No17 | CCDC99 | Unknown | NM_017785 |
| 210762_s_at | Seq Id No4 | DLC1 | A candidate tumor suppressor gene | NM_182643.2 |
| 205308_at | Seq ID No7 | FAM164A | Unknown | NM_016010 |
| 46142_at | Seq ID No18 | LMF1 | Maturation of specific proteins in the | NM_022773 |
| | | | endoplasmic reticulum | |
| 204524_at | Seq ID No19 | PDPK1 | Cell signal protein | NM_002613 |
| 222078_at | Seq ID No20 | PKLR | Pyruvate kinase | NM_000298 |
| | | | | NM_181871 |
| 219808_at | Seq ID No21 | SCLY | Catalyzes the decomposition of L- | NM_016510 |
| | | | selenocysteine to L-alanine and | |
| | | | elemental selenium | |
| 209420_s_at | Seq ID No22 | SMPD1 | Converts sphingomyelin to ceramide | NM_000543 |
| 208855_s_at | Seq ID No23 | STK24 | Protein kinase | NM_001032296 |
| 208775_at | Seq ID No24 | XPO1 | Nuclear protein transport | NM_003400 |
| 218833_at | Seq ID No25 | ZAK | Cell signal protein | NM_016653 |

TABLE 3
The identified 16 prognostic signature genes for non-small cell lung cancer

| | | Gene | | |
|---|---|---|---|---|
| Probe Set | Name | Symbol | Function | Sequence ID |
| 212041_at | Seq ID No16 | ATP6V0D1 | Atpase | NM_004691 |
| 206150_at | Seq ID No2 | CD27 | B-cell activation and immunoglobulin | NM_001242 |
| | | | synthesis; signaling transduction | |
| 210762_s_at | Seq ID No4 | DLC1 | A candidate tumor suppressor gene | NM_182643.2 |
| 211603_s_at | Seq ID No6 | ETV4 | Cellular movement | NM_001079675 |
| 211327_x_at | Seq ID No8 | HFE | Iron absorption | NM_000410 |
| 46142_at | Seq ID No18 | LMF1 | Maturation of specific proteins in the | NM_022773 |
| | | | endoplasmic reticulum | |
| 204524_at | Seq ID No19 | PDPK1 | Cell signal protein | NM_002613 |
| 222078_at | Seq ID No20 | PKLR | Pyruvate kinase | NM_000298 |
| | | | | NM_181871 |
| 205171_at | Seq ID No10 | PTPN4 | Cell growth, differentiation, mitotic | NM_002830 |
| | | | cycle, and oncogenic transformation | |
| 219808_at | Seq ID No21 | SCLY | Catalyzes the decomposition of L- | NM_016510 |
| | | | selenocysteine to L-alanine and | |
| | | | elemental selenium | |
| 209420_s_at | Seq ID No22 | SMPD1 | Converts sphingomyelin to ceramide | NM_000543 |
| 208855_s_at | Seq ID No23 | STK24 | Protein kinase | NM_001032296 |
| 201107_s_at | Seq ID No11 | THBS1 | Cell-to-cell and cell-to-matrix | NM_003246 |
| | | | interactions. | |
| 201581_at | Seq ID No13 | TXNDC13 | Cell redox homeostasis, electron | NM_021156 |
| | | (TMX4) | transport chain | |
| 208775_at | Seq ID No24 | XPO1 | Nuclear protein transport | NM_003400 |
| 218833_at | Seq ID No25 | ZAK | Cell signal protein | NM_016653 |

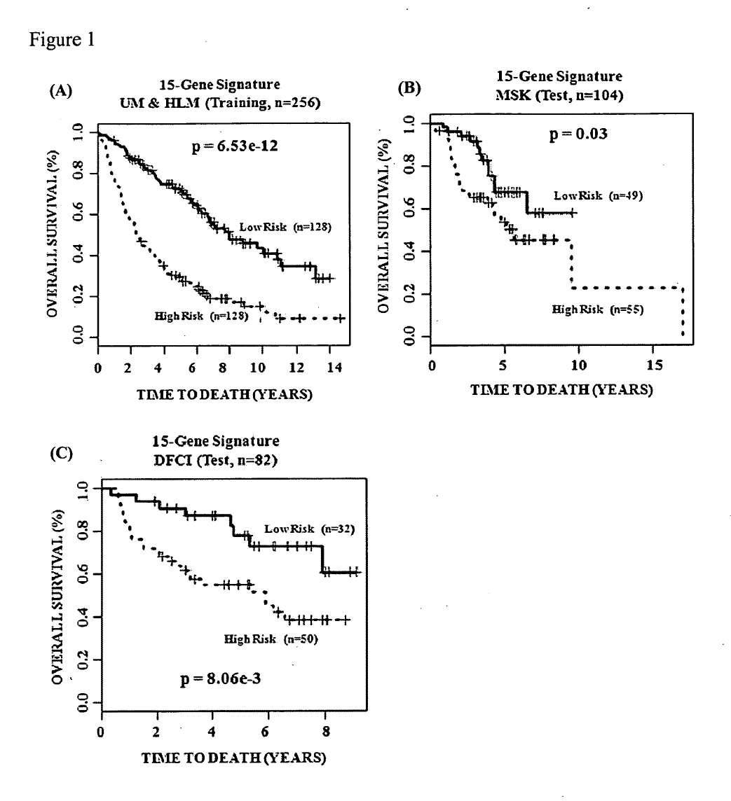

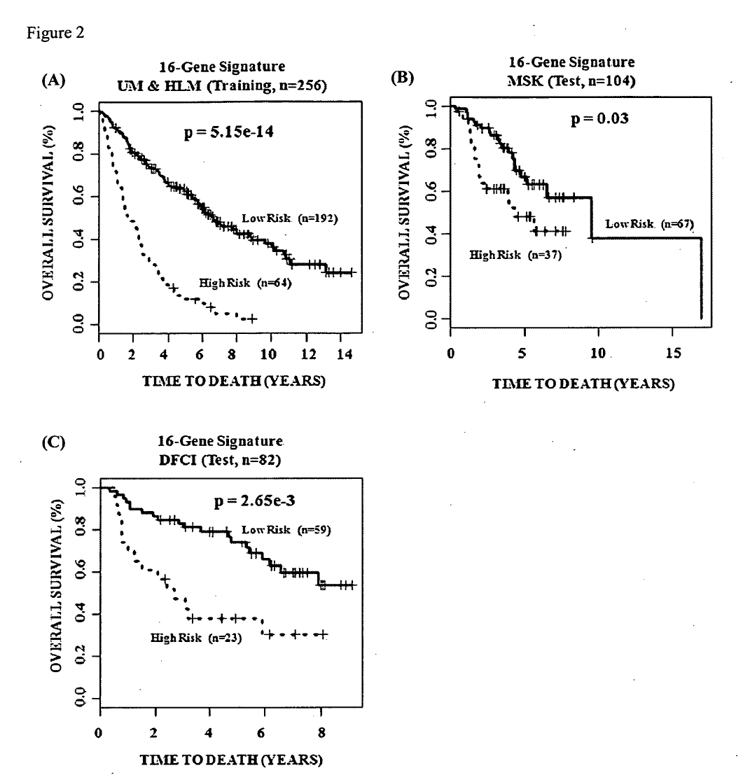

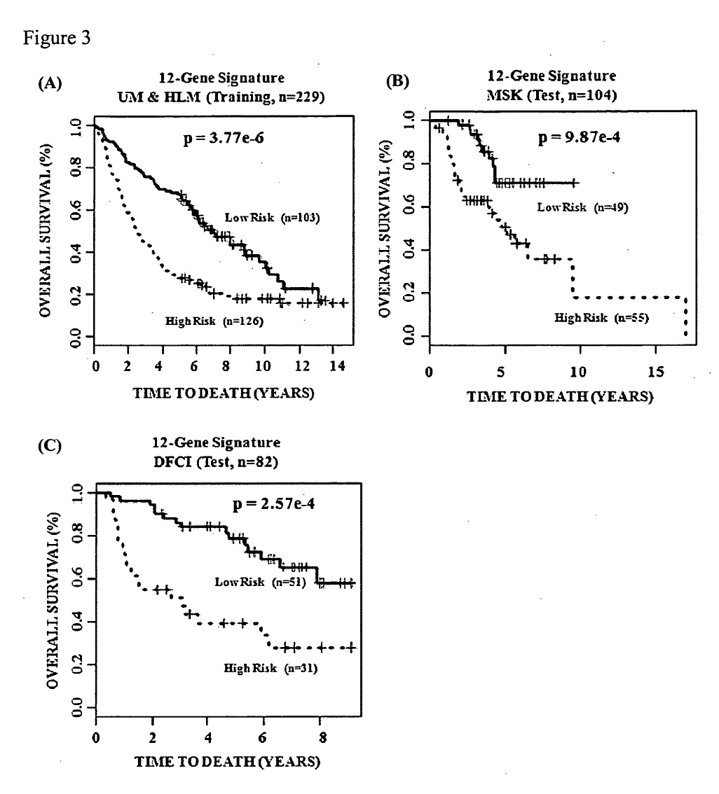

To evaluate overall survival prediction, classifier was constructed on training cohort (n=256) and validated in two independent test sets (n=104, n=84) from Shedden et al. (1). The expression profiles of the 15-gene signature on the training cohort were fitted into a Cox proportional hazard model as covariates. Then, using median risk score (−1.79) from training patients as the cutoff, patients with risk scores less than the cutoff value would be classified into low-risk group; otherwise, patients would be classified into high risk groups. Risk scores of patients in both test sets would be computed using regression coefficient of each signature gene from the Cox model fitted with training data. Same classification scheme would be applied to stratify patients in test sets into low- or high-risk groups. The prediction model accurately stratified patients into two distinct risk groups (log-rank P<0.03, Kaplan-Meier analysis) (FIG. 1) with significantly distinct post-operative survival (log-rank P<6.53e−12) in training set (A) with respectable tumor stages. The model also stratified patients with all tumor stages into two significantly distinct prognostic groups (log-rank P<0.03) in both test sets (B, C) independently. With similar approach, another prediction model was constructed using Cox proportional hazard model with the 16-gene signature as covariates. In the 16-gene prognostic model, 75^{th} percentile of the risk score from training cohort (1.57) was used as the cutoff to stratify patients. The 16-gene prognostic model also correctly stratified patients in training and test sets into two distinct risk groups (log-rank P<0.03, Kaplan-Meier analysis) (FIG. 2). The model correctly stratified patients into two prognostic groups with significantly distinct post-operative survival (log-rank P<5.15e−14) in training set (A) with respectable tumor stages. The model also stratified patients with all stages into two significantly distinct prognostic groups (log-rank P<0.03) in both test sets (B, C) independently. With the 12-gene signature, Naïve Bayes classifier was used to construct the model to predict overall survival in lung cancer patients. In training cohort, survival status for each patient was defined based on 5-year survival status: patients who survived 5 years or longer were defined as low-risk patients (n=104); patients who died in less than 5-year time were defined as high-risk patients (n=125); all other cases (n=27) were considered censored cases and excluded from training cohort. 10-fold cross validation was used in evaluating the performance of the model in training cohort. The trained Naïve Bayes classifier computed posterior probability of both low- and high-risk groups for each patient and classified the patient into the group with greater posterior probability. In other words, based on posterior probability of high-risk group alone, patients would be classified into high-risk group if the value is greater than 0.5; or low-risk group otherwise. Using the trained Naïve Bayes classifier, high-risk posteriors for each patient in two test sets was computed and used to classify patients into high- or low-risk group at the 0.5 cutoff. After obtaining the predicted outcomes, Kaplan-Meier analysis was carried out to study the strength of prediction produced by the model with respect to the survival data of patients. The model showed accurate prediction as it stratified patients into two significantly different survival groups (log-rank P<0.001, Kaplan-Meier analysis) (FIG. 3) with distinct post-operative survival (log-rank P<3.77e−6) in training set (A) with all stages of 5-year survival using 10-fold cross validation. The model also stratified patients with all stages into two significantly distinct survival groups (log-rank P<0.001) in both test sets (B, C) independently.

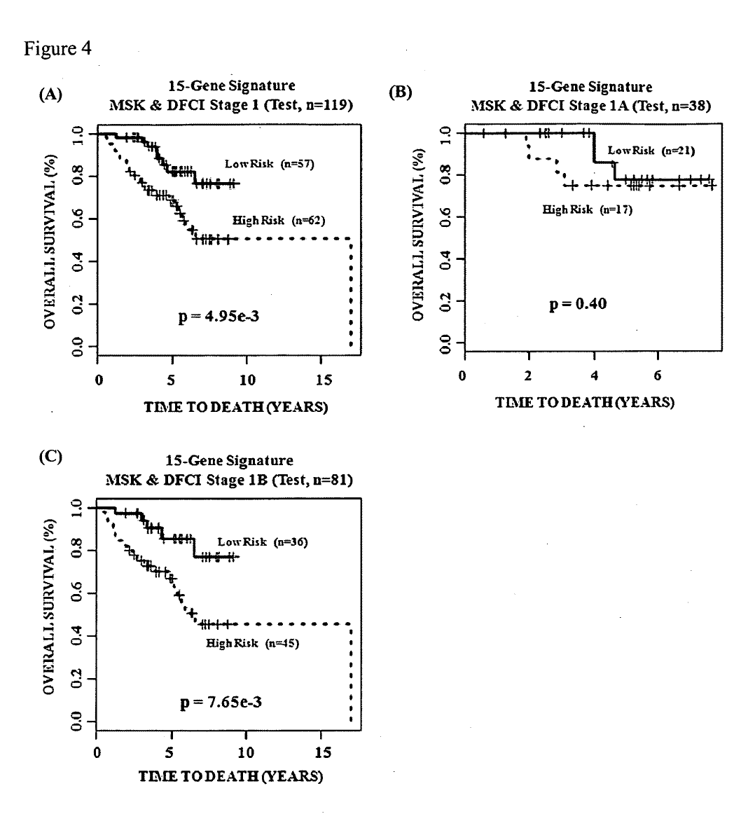

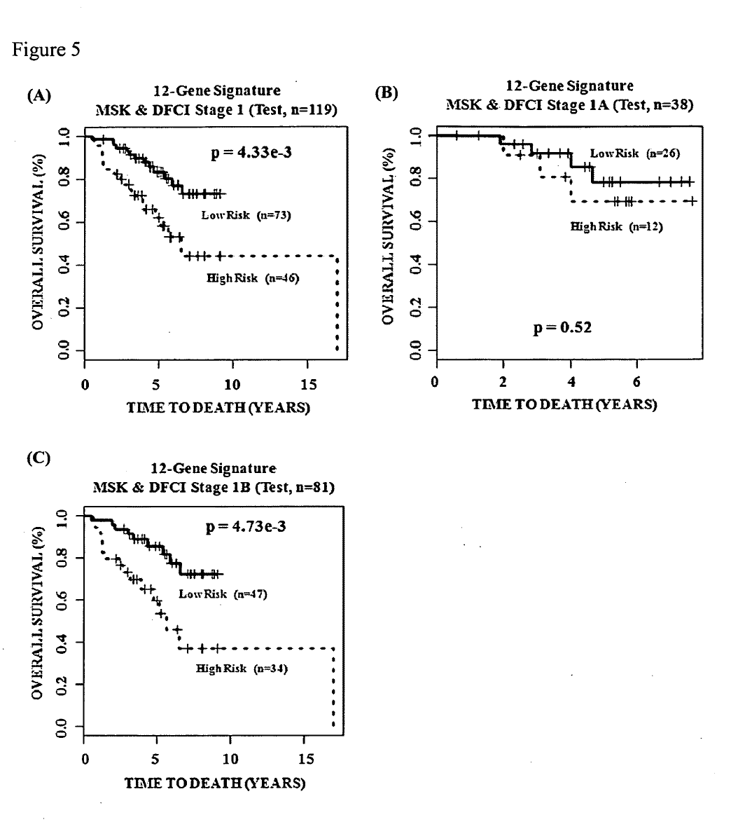

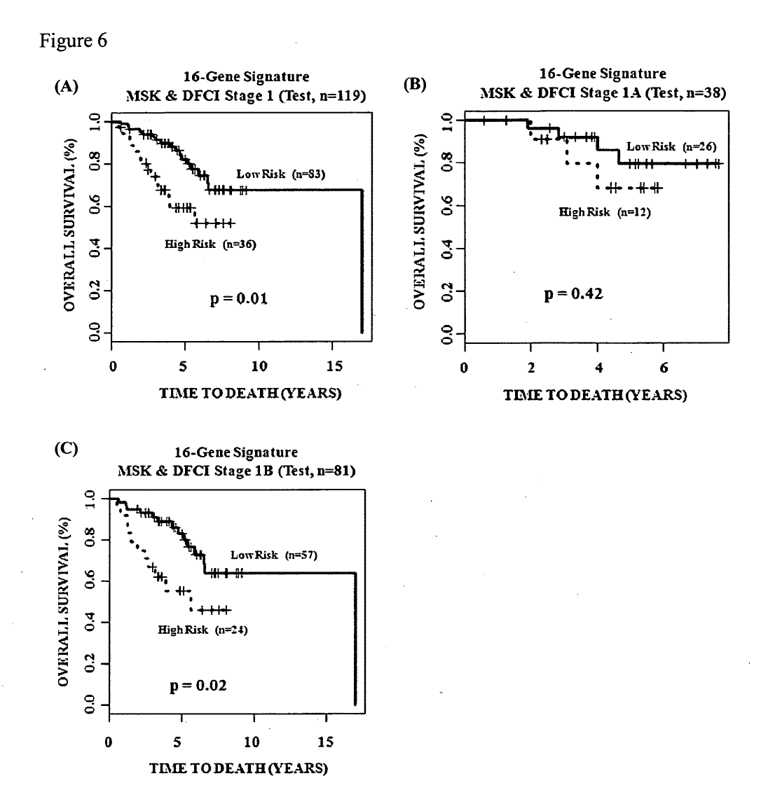

Previous studies (1;2) showed that current lung cancer prognosis based on AJCC tumor stage was not accurate enough; especially in early stages. The model's prediction performance on early stage patients was needed. With models constructed using all patient samples in training cohort as discussed in section previously, predictions on stage 1, stage 1A, and stage 1B patients in test sets were evaluated independently using Kaplan-Meier analysis. Due to small sample size samples in both test sets for each stage were combined. The constructed 15-, 12-, and 16-gene models gave accurate prediction (log-rank P<0.02) on stage 1 patients and stage 1B patients (FIG. 4A, 4C, 5A, 5C, 6A, 6C) but not on stage 1A patients (FIG. 4B, 5B, 6B). The model stratified stage 1 patients (A) and stage 1B patients (C) into two significantly different survival risk-groups (log-rank P<0.005). The model in FIG. 6 stratified stage 1 patients (A) and stage 1B patients (C) into two significantly different survival risk-groups (log-rank P<0.02).

In order to confirm the prognostic power of the model on overall survival of lung cancer, the relationships of the model's predictions and various clinical covariates to the patients' survival outcome using multivariate Cox analysis was studied. In the assessment, predicted risk scores were used in the 15- and 16-gene model and the predicted high-risk posterior probabilities were used in the 12-gene model. Two multivariate Cox analyses were carried out. The first analysis compared the model's performance with major clinical covariates known of their strong associations with lung cancer patients' overall survival (Table 4). The second multivariate Cox analysis included all clinical covariates available in the dataset used (Table 5). In both analyses, 15-, 12-, and 16-gene showed that they could accurately predict the risk-level in lung cancer patients (HR>=1.9, P-value <0.01). Lymph node metastasis status appeared to be the best covariates associated with lung cancer.

TABLE 4
Multivariate Cox proportional analysis of major clinical covariates
Gender, Age, Lymph node metastasis, Tumor size, and 15-gene,
12-gene, 16-gene predictions in relation to the likelihood of high risk.*

| | | |
|---|---|---|
| Variable | P value | Hazard Ratio (95% CI)^{ψ} |

Analysis with clinical covariates only

| | | | |
|---|---|---|---|
| Gender (Male) | 0.06 | 1.29 | (0.99-1.67) |
| Age at diagnosis (>60) | 8.00E−04 | 1.69 | (1.24-2.30) |
| Lymph node metastasis | 6.20E−14 | 2.72 | (2.09-3.53) |
| Tumor size (>3 cm) | 3.50E−03 | 1.54 | (1.15-2.05) |

Analysis with predicted high-risk posteriors (12-gene model)

| | | | |
|---|---|---|---|
| Gender (Male) | 0.16 | 1.21 | (0.93-1.57) |
| Age at diagnosis (>60) | 6.15E−03 | 1.54 | (1.13-2.10) |
| Lymph node metastasis | 3.88E−11 | 2.43 | (1.87-3.16) |
| Tumor size (>3 cm) | 0.25 | 1.19 | (0.88-1.61) |
| Probability to be high-risk | 1.66E−11 | 3.86 | (2.60-5.72) |

Analysis with predicted risk scores (15-gene model)

| | | | |
|---|---|---|---|
| Gender (Male) | 0.03 | 1.33 | (1.02-1.72) |
| Age at diagnosis (>60) | 6.66E−04 | 1.71 | (1.26-2.33) |
| Lymph node metastasis | 4.05E−11 | 2.44 | (1.87-3.18) |
| Tumor size (>3 cm) | 0.16 | 1.24 | (0.92-1.67) |
| 15-gene predicted risk scores | 3.60E−14 | 2.01 | (1.68-2.40) |

Analysis with predicted risk scores (16-gene model)

| | | | |
|---|---|---|---|
| Gender (Male) | 0.02 | 1.36 | (1.04-1.77) |
| Age at diagnosis (>60) | 0.00 | 1.57 | (1.15-2.14) |
| Lymph node metastasis | 1.86E−11 | 2.45 | (1.89-3.18) |
| Tumor size (>3 cm) | 0.22 | 1.20 | (0.90-1.62) |
| 16-gene predicted risk scores | 3.77E−15 | 1.90 | (1.62-2.22) |

*Age at diagnosis was a binary variable (0 for <60 years old and 1 otherwise); lymph node metastasis was a binary variable (0 for N0 stage and 1 for all other N-stages or unknown); tumor size was a binary variable (0 for <3 m in greatest dimension and 1 for all other sizes or unknown).
^{ψ}denotes confidence interval.

TABLE 5
Multivariate Cox proportional analysis of all available clinical
covariates and 15-gene, 12-gene, 16-gene predictions to death
in relation to the likelihood of high risk.*

| | | |
|---|---|---|
| Variable | P value | Hazard Ratio (95% CI)^{ψ} |

Analysis with clinical covariates only

| | | | |
|---|---|---|---|
| Gender (Male) | 0.06 | 1.31 | (0.99-1.74) |
| Age at diagnosis (>60) | 0.00 | 1.71 | (1.25-2.32) |
| Lymph node metastasis | 0.00 | 2.79 | (2.14-3.64) |
| Tumor size (>3 cm) | 0.00 | 1.57 | (1.17-2.10) |
| Race | | | |
| Others/Unknown | 0.76 | 0.88 | (0.38-2.05) |
| White | 0.72 | 1.16 | (0.51-2.63) |
| Tumor Grade | | | |
| Moderately differentiate | 0.38 | 0.83 | (0.54-1.27) |
| Poorly differentiate | 0.80 | 0.95 | (0.61-1.47) |
| Smoking History | | | |
| Smokers | 0.40 | 1.23 | (0.76-1.99) |
| Unknown | 0.25 | 1.39 | (0.80-2.41) |

Analysis with predicted high-risk posteriors (12-gene model)

| | | | |
|---|---|---|---|
| Gender (Male) | 0.15 | 1.23 | (0.93-1.63) |
| Age at diagnosis (>60) | 0.01 | 1.51 | (1.11-2.07) |
| Lymph node metastasis | 1.53E−11 | 2.50 | (1.92-3.27) |
| Tumor size (>3 cm) | 0.19 | 1.22 | (0.90-1.66) |
| Race | | | |
| Others/Unknown | 0.90 | 1.05 | (0.45-2.47) |
| White | 0.62 | 1.23 | (0.54-2.79) |
| Tumor differentiation | | | |
| Moderately differentiate | 0.24 | 0.78 | (0.51-1.19) |
| Poorly differentiate | 0.14 | 0.71 | (0.45-1.12) |
| Smoking History | | | |
| Smokers | 0.42 | 1.22 | (0.76-1.96) |
| Unknown | 0.55 | 1.19 | (0.68-2.08) |
| Probability to be high-risk | 2.38E−11 | 4.02 | (2.67-6.04) |

Analysis with predicted risk scores (15-gene model)

| | | | |
|---|---|---|---|
| Gender (Male) | 0.08 | 1.28 | (0.97-1.69) |
| Age at diagnosis (>60) | 9.04E−04 | 1.69 | (1.24-2.31) |
| Lymph node metastasis | 1.54E−11 | 2.51 | (1.92-3.28) |
| Tumor size (>3 cm) | 0.08 | 1.31 | (0.97-1.77) |
| Race | | | |
| Others/Unknown | 0.60 | 0.80 | (0.34-1.86) |
| White | 0.97 | 1.01 | (0.45-2.31) |
| Tumor differentiation | | | |
| Moderately differentiate | 0.30 | 0.80 | (0.52-1.22) |
| Poorly differentiate | 0.23 | 0.76 | (0.49-1.19) |
| Smoking History | | | |
| Smokers | 0.23 | 1.34 | (0.83-2.15) |
| Unknown | 0.06 | 1.69 | (0.97-2.94) |
| 15-gene predicted risk scores | 3.18E−14 | 2.06 | (1.71-2.48) |

Analysis with predicted risk scores (16-gene model)

| | | | |
|---|---|---|---|
| Gender (Male) | 0.05 | 1.33 | (1.01-1.76) |
| Age at diagnosis (>60) | 0.01 | 1.55 | (1.14-2.12) |
| Lymph node metastasis | 6.93E−12 | 2.52 | (1.94-3.29) |
| Tumor size (>3 cm) | 0.15 | 1.25 | (0.92-1.68) |
| Race | | | |
| Others/Unknown | 0.32 | 0.65 | (0.28-1.52) |
| White | 0.66 | 0.83 | (0.36-1.89) |
| Tumor differentiation | | | |
| Moderately differentiate | 0.29 | 0.79 | (0.52-1.22) |
| Poorly differentiate | 0.32 | 0.80 | (0.51-1.25) |
| Smoking History | | | |
| Smokers | 0.34 | 1.26 | (0.78-2.03) |
| Unknown | 0.10 | 1.59 | (0.91-2.78) |
| 16-gene predicted risk scores | 5.22E−15 | 1.94 | (1.64-2.29) |

*Age at diagnosis was a binary variable (0 for <60 years old and 1 otherwise); lymph node metastasis was a binary variable (0 for N0 stage and 1 for all other N-stages or unknown); tumor size was a binary variable (0 for <3 m in greatest dimension and 1 for all other sizes or unknown); race was a categorical variable of 3 categories (African American [as the reference group], White, and Others [composed of Asian (5), Hawaiian or Pacific Islander (1), and unknown]); tumor grade was categorical variable of 3 categories (Well [as the reference group], Moderately, and Poorly differentiate); Smoking history was a categorical variable of 3 categories (Non-smokers, Smokers, and Unknown).
^{ψ}denotes confidence interval.

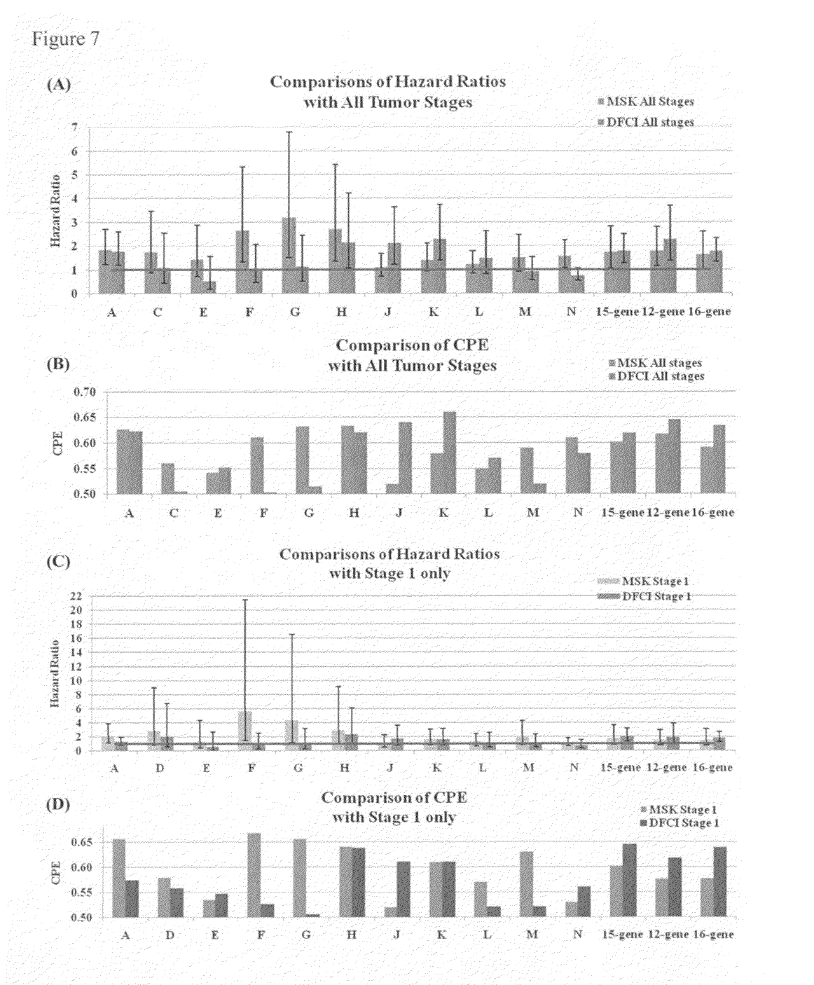

The study was carried out using published data from Shedden et al (1). They had modeled multiple molecular classifiers and the best model was “method A”. Estimated hazard ratio and concordance probability estimate (CPE) for the risk scores produce by the models were used as assessment metrics. The hazard ratio and CPE from their models with the 15-gene, 12-gene, and 16-gene model were compared. For the 12-gene model, instead of predicted risk scores from the model, predicted posterior probability to high-risk group were used in the assessment. Table 6 presents a summary of various gene selections and classification methods of molecular classifiers compared. Comparison results showed that all three models were as good as the best model and other models presented by Shedden et al in patient samples with all tumor stages (FIG. 7A, 7B) and patient samples with stage 1 tumor only (FIG. 7C, 7D). The models identified using dataset from Shedden (Shedden et al, 2008) in terms of hazard ratio (A, C) and concordance probability estimate (CPE) (B, D) on patients in all stages (A, B) and stage 1 (C, D) of lung cancer. The error bars in (A) and (C) represent 95% confidence interval of the hazard ratio.

TABLE 6
Summary of gene selection and classification methods of molecular classifiers
compared in FIG. 7. Gene signatures A-N were evaluated in (Shedden et al, 2008).

| Molecular | Number of | | |
|---|---|---|---|
| Classifier* | signature genes | Gene selection method(s) | Classification method(s) |
| Shedden A | ~9591 Genes | Clustering analysis | Ridged Cox proportional |
| | | | hazard model |
| Shedden C | 23 Genes | SAM, Maximizing Chi-Square | Binary Tree-Structured |
| | | analysis (MCA, univariate Cox | Vector Quantization |
| | | model and k-mean clustering) | (BTSVQ) |
| Shedden D | 37 Genes | SAM, Maximizing Chi-Square | Binary Tree-Structured |
| | | analysis (MCA, univariate Cox | Vector Quantization |
| | | model and k-mean clustering) | (BTSVQ) |
| Shedden E | 1 Gene | Gene Expression Fold Change | Post-hoc split of expression |
| | | | of one gene |
| Shedden F | 42 Genes | Univariate Cox Model | Principle Components and |
| | | | Cox Model |
| Shedden G | 38 Genes | Univariate Cox Model | Principle Components and |
| | | | Cox Model |
| Shedden H | 252 Genes | Scoring and filtering on set of | Majority vote |
| | | mitosis genes | |
| Shedden J | 5 Genes | Univariate Cox model (Chen et | Ridged Cox proportional |
| | | al, NEJM 07) | hazard model |
| Shedden K | 16 Genes | Univariate Cox model (Chen et | Ridged Cox proportional |
| | | al, NEJM 07) | hazard model |
| Shedden L | 9 Genes | Principal Components (Potti et | Ridged Cox proportional |
| | (from 80 Genes) | al, NEJM 06) | hazard model |
| Shedden M | 45 Genes | Principal Components (Potti et | Ridged Cox proportional |
| | (from 80 Genes) | al, NEJM 06) | hazard model |
| Shedden N | 80 Genes | Principal Components (Potti et | Ridged Cox proportional |
| | | al, NEJM 06) | hazard model |
| 15-gene | 15 Genes | t-test, RELIEFF, | Cox proportional hazard |
| | | | model |
| 12-gene | 12 Genes | t-test, SAM, RELIEFF | Naïve Bayes |
| 16-gene | 16 Genes | t-test, SAM, RELIEFF, | Cox proportional hazard |
| | | biological functions | model |

*Gene signatures A-H were identified in (Shedden et al, 2008). Gene signatures J and K were identified in (Chen et al, 2007). Gene signatures L, M, and N were identified in (Potti et al, 2006).

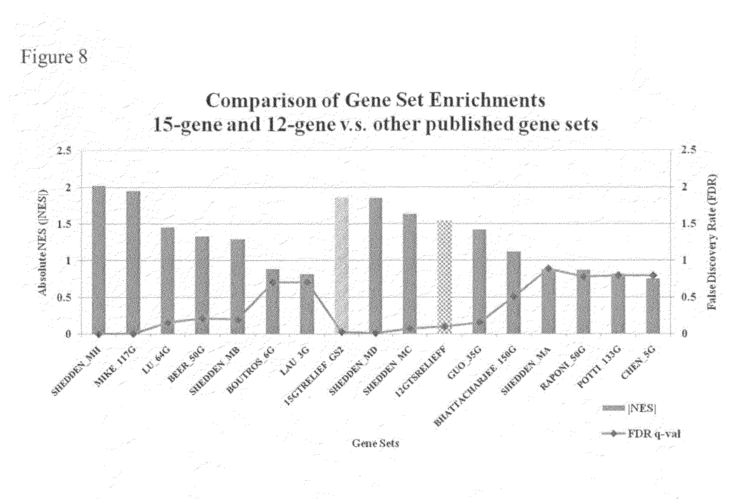

In order to compare these signatures to various prognostic gene signature proposed in the literature over the years (1-10) Gene Set Enrichment Analysis (GSEA) was used to assess the associations of expression levels of these genes to 5-year postoperative survival. On all 442 samples that were used in the study, normalized enrichment score (NES) and its corresponding false discovery rate (FDR) were obtained from GSEA and evaluated. In general, gene set with extreme NES and relatively low FDR is desired as it indicates that the gene set expresses diversely with respect to the survival outcome and the finding is of relatively low possibility that the phenomenon occurs by chance. In comparison to 14 published gene signatures (Table 7), 15-gene and 12-gene signatures exhibited high associations to patient-group whose survival is longer than 5 years with significantly low FDR (NES>=1.5; FDR<0.10). False discovery rate (FDR q-value) and the absolute of normalized enrichment score (|NESJ|) computed for each signatures from the GSEA are compared in FIG. 8.

TABLE 7
14 published lung cancer molecular biomarkers included in GSEA study (FIG. 8).

| | | | No. of | No. of Genes |
|---|---|---|---|---|
| Signature Name | | Publication | Signature | matched in GSEA |
| (GSEA) | First Author | PubMed ID | Genes/Probes | (By gene symbol) |
| Beer_50 g | Beer, DG | PMID: 12118244 | 50 | 45 |
| Bhattachaijee_150 g | Bhattacharjee, A | PMID: 11707567 | 150 | 130 |
| Boutros_6 g | Boutros, PC | PMID: 19196983 | 6 | 6 |
| Chen_5 g | Chen, HY | PMID: 17202451 | 5 | 5 |
| Guo_35 g | Guo, L | PMID: 16740756 | 35 | 34 |
| Lau_3 g | Lau, SK | PMID: 18065728 | 3 | 3 |
| Lu_64 g | Lu, Y | PMID: 17194181 | 64 | 62 |
| Potti_133 g | Potti, A | PMID: 16899777 | 133 | 129 |
| Raponi_50 g | Raponi, M | PMID: 16885343 | 50 | 44 |
| Shedden_MA | Shedden, K | PMID: 18641660 | 13830 | 8319 |
| Shedden_MB | Shedden, K | PMID: 18641660 | 52 | 50 |
| Shedden_MC | Shedden, K | PMID: 18641660 | 26 | 23 |
| Shedden_MD | Shedden, K | PMID: 18641660 | 42 | 34 |
| Shedden_MH | Shedden, K | PMID: 18641660 | 313 | 244 |

Biological aspect of the gene signatures to lung cancer based on curated molecular interactions to other genes were studied using Ingenuity Pathway analysis (IPA). Core analysis on IPA was performed to reveal in which regulatory networks the set of signature genes are highly involved. The 12-gene signature was shown to have interactions to major cancer signaling pathways such as TNF and AKT (FIG. 9). The 15-gene also involved in cancer signaling pathways ERBB2 (FIG. 10).

Curated relationships among the signature genes and 13 prominent lung cancer hallmarks (EGF, EGFR, KRAS, MET, RB1, TP53, E2F1, E2F2, E2F3, E2F4, E2F5, AKT1, TNF) were retried using Pathway Studio. Most of the signature genes are directly or indirectly related to the lung cancer hallmarks in various processes, ranging from regulations to molecular transport (FIG. 11). Interactions among the hallmarks were removed to simplify the figure and have a clearer view on interactions of signature genes to hallmarks.

Biological functions from curated database between 15- and 12-gene signatures were studied using IPA. In addition to sharing two common genes between the two signatures, they shared most biological functions, especially functions related to diseases and disorders (Table 8).

TABLE 8
Comparison of biological functions from curated database between 12-gene
signature and 15-gene signature

| Category | Category | 12-gene | 15-gene | Common |
|---|---|---|---|---|
| Diseases and | Cancer | | | ✓ |
| Disorders | Cardiovascular Disease | | ✓ | |
| | Connective Tissue Disorders | | ✓ | |
| | Dermatological Diseases and Conditions | | ✓ | |
| | Genetic Disorder | | | ✓ |
| | Hematological Disease | | | ✓ |
| | Hepatic System Disease | | | ✓ |
| | Immunological Disease | | | ✓ |
| | Infection Mechanism | ✓ | | |
| | Inflammatory Disease | | ✓ | |
| | Inflammatory Response | | ✓ | |
| | Metabolic Disease | | | ✓ |
| | Neurological Disease | | | ✓ |
| | Reproductive System Disease | | | ✓ |
| | Respiratory Disease | | | ✓ |
| | Skeletal and Muscular Disorders | | ✓ | |
| Molecular and | Amino Acid Metabolism | | | ✓ |
| Cellular | Antigen Presentation | | ✓ | |
| Functions | Carbohydrate Metabolism | | ✓ | |
| | Cell Cycle | | ✓ | |
| | Cell Death | | | ✓ |
| | Cell Morphology | | ✓ | |
| | Cell Signaling | | | ✓ |
| | Cell-To-Cell Signaling and Interaction | | ✓ | |
| | Cellular Assembly and Organization | | | ✓ |
| | Cellular Compromise | | ✓ | |
| | Cellular Development | | | ✓ |
| | Cellular Function and Maintenance | | | ✓ |
| | Cellular Growth and Proliferation | | | ✓ |
| | Cellular Movement | | | ✓ |
| | DNA Replication, Recombination, and | ✓ | | |
| | Repair | | | |
| | Drug Metabolism | | ✓ | |
| | Gene Expression | ✓ | | |
| | Lipid Metabolism | | | ✓ |
| | Molecular Transport | | | ✓ |
| | Nucleic Acid Metabolism | | ✓ | |
| | Post-Translational Modification | | | ✓ |
| | Protein Synthesis | | ✓ | |
| | Protein Trafficking | | ✓ | |
| | RNA Trafficking | ✓ | | |
| | Small Molecule Biochemistry | | | ✓ |
| Physiological | Cardiovascular System Development and | | ✓ | |
| System | Function | | | |
| Development | Cell-mediated Immune Response | | ✓ | |
| and Function | Hematological System Development and | | ✓ | |
| | Function | | | |
| | Immune Cell Trafficking | | ✓ | |
| | Nervous System Development and | | ✓ | |
| | Function | | | |
| | Organ Development | | ✓ | |
| | Skeletal and Muscular System | | | ✓ |
| | Development and Function | | | |
| | Tissue Development | | ✓ | |
| | Tumor Morphology | | ✓ | |
| | Visual System Development and | | ✓ | |
| | Function | | | |

Various subsets of the prognostic signature genes from the 15-, 12-, and 16-gene signatures predict overall survival of lung cancer patients with all tumor stages or stage 1 tumors only. By fitting the expressions profiles of the genes into Cox proportional hazard model as covariates, classifiers are constructed to predict overall survival in lung cancer patients in training data from Shedden et al (1). The constructed models were then validated in test sets from Shedden et al (1).

There are 5 genes (Table 9) predicted overall survival of lung cancer patients in all stages, patients in stage 1, and patients in stage 1B from Shedden et al (1).

TABLE 9
5 of the 25 prognostic signature genes predict overall survival
in lung cancer patients from Shedden et al (1) with all tumor stages,
stage 1 tumors, and stage 1B tumors.

| Gene Symbol | Sequence ID |
|---|---|
| DKFZp434H1419 | |
| FAM164A | NM_016010 |
| HFE | NM_000410 |
| PKLR | NM_000298 |
| UBA6 | NM_018227 |

There are 6 genes (Table 10) predicted overall survival of lung cancer patients in all stages, patients in stage 1, and patients in stage 1B from Shedden et al (1).

TABLE 10
6 of the 25 prognostic signature genes predict overall survival
in lung cancer patients from Shedden et al (1) with all tumor stages,
stage 1 tumors, and stage 1B tumors.

| Gene Symbol | Sequence ID |
|---|---|
| DKFZp434H1419 | |
| DLC1 | NM_182643.2 |
| FAM164A | NM_016010 |
| HFE | NM_000410 |
| PKLR | NM_000298 |
| UBA6 | NM_018227 |

There are 7 genes (Table 11) predicted overall survival of lung cancer patients in all stages, patients in stage 1, and patients in stage 1B from Shedden et al (1).

TABLE 11
7 of the 25 prognostic signature genes predict overall survival
in lung cancer patients from Shedden et al (1) with all tumor stages,
stage 1 tumors, and stage 1B tumors.

| Gene Symbol | Sequence ID |
|---|---|
| DKFZp434H1419 | |
| DLC1 | NM_182643.2 |
| FAM164A | NM_016010 |
| HFE | NM_000410 |
| PKLR | NM_000298 |
| THBS1 | NM_003246 |
| UBA6 | NM_018227 |

There are 8 genes (Table 12) predicted overall survival of lung cancer patients in all stages, patients in stage 1, and patients in stage 1B from Shedden et al (1).

TABLE 12
8 of the 25 prognostic signature genes predict overall survival
in lung cancer patients from Shedden et al (1) with all tumor stages,
stage 1 tumors, and stage 1B tumors.

| Gene Symbol | Sequence ID |
|---|---|
| CD27 | NM_001242 |
| DKFZp434H1419 | |
| DLC1 | NM_182643.2 |
| FAM164A | NM_016010 |
| HFE | NM_000410 |
| PKLR | NM_000298 |
| THBS1 | NM_003246 |
| UBA6 | NM_018227 |

There are 9 genes (Table 13) predicted overall survival of lung cancer patients in all stages, patients in stage 1, and patients in stage 1B from Shedden et al (1).

TABLE 13
9 of the 25 prognostic signature genes predict overall survival
in lung cancer patients from Shedden et al (1) with all tumor stages,
stage 1 tumors, and stage 1B tumors.

| Gene Symbol | Sequence ID |
|---|---|
| CD27 | NM_001242 |
| DKFZp434H1419 | |
| DLC1 | NM_182643.2 |
| ETV4 | NM_001079675 |
| FAM164A | NM_016010 |
| HFE | NM_000410 |
| PKLR | NM_000298 |
| THBS1 | NM_003246 |
| UBA6 | NM_018227 |

There are 10 genes (Table 14) predicted overall survival of lung cancer patients in all stages, patients in stage 1, and patients in stage 1B from Shedden et al (1).

TABLE 14
10 of the 25 prognostic signature genes predict overall survival
in lung cancer patients from Shedden et al (1) with all tumor stages,
stage 1 tumors, and stage 1B tumors.

| Gene Symbol | Sequence ID |
|---|---|
| CD27 | NM_001242 |
| DKFZp434H1419 | |
| DLC1 | NM_182643.2 |
| ETV4 | NM_001079675 |
| FAM164A | NM_016010 |
| HFE | NM_000410 |
| PKLR | NM_000298 |
| THBS1 | NM_003246 |
| UBA6 | NM_018227 |
| ZAK | NM_016653 |

There are 11 genes (Table 15) predicted overall survival of lung cancer patients in all stages, patients in stage 1, and patients in stage 1B from Shedden et al (1).

TABLE 15
11 of the 25 prognostic signature genes predict overall survival
in lung cancer patients from Shedden et al (1) with all tumor stages,
stage 1 tumors, and stage 1B tumors.

| Gene Symbol | Sequence ID |
|---|---|
| ANKHD1 | NM_017747 |
| CD27 | NM_001242 |
| DKFZp434H1419 | |
| DLC1 | NM_182643.2 |
| ETV4 | NM_001079675 |
| FAM164A | NM_016010 |
| HFE | NM_000410 |
| PKLR | NM_000298 |
| THBS1 | NM_003246 |
| UBA6 | NM_018227 |
| ZAK | NM_016653 |

There are 12 genes (Table 16) predicted overall survival of lung cancer patients in all stages, patients in stage 1, and patients in stage 1B from Shedden et al (1).

TABLE 16
12 of the 25 prognostic signature genes predict overall survival in
lung cancer patients from Shedden et al (1) with all tumor stages,
stage 1 tumors, and stage 1B tumors.

| Gene Symbol | Sequence ID |
|---|---|
| ANKHD1 | NM_017747 |
| CCDC99 | NM_017785 |
| CD27 | NM_001242 |
| DKFZp434H1419 | |
| DLC1 | NM_182643.2 |
| ETV4 | NM_001079675 |
| FAM164A | NM_016010 |
| HFE | NM_000410 |
| PKLR | NM_000298 |
| THBS1 | NM_003246 |
| UBA6 | NM_018227 |
| ZAK | NM_016653 |

There are 13 genes (Table 17) predicted overall survival of lung cancer patients in all stages, patients in stage 1, and patients in stage 1B from Shedden et al (1).

TABLE 17
13 of the 25 prognostic signature genes predict overall survival in
lung cancer patients from Shedden et al (1) with all tumor stages,
stage 1 tumors, and stage 1B tumors.

| Gene Symbol | Sequence ID |
|---|---|
| ANKHD1 | NM_017747 |
| ATP6V0D1 | NM_004691 |
| CCDC99 | NM_017785 |
| CD27 | NM_001242 |
| DKFZp434H1419 | |
| DLC1 | NM_182643.2 |
| ETV4 | NM_001079675 |
| FAM164A | NM_016010 |
| HFE | NM_000410 |
| PKLR | NM_000298 |
| THBS1 | NM_003246 |
| UBA6 | NM_018227 |
| ZAK | NM_016653 |

There are 14 genes (Table 18) predicted overall survival of lung cancer patients in all stages, patients in stage 1, and patients in stage 1B from Shedden et al (1).

TABLE 18
14 of the 25 prognostic signature genes predict overall survival in
lung cancer patients from Shedden et al (1) with all tumor stages,
stage 1 tumors, and stage 1B tumors.

| Gene Symbol | Sequence ID |
|---|---|
| ANKHD1 | NM_017747 |
| ATP6V0D1 | NM_004691 |
| CCDC99 | NM_017785 |
| CD27 | NM_001242 |
| DKFZp434H1419 | |
| DLC1 | NM_182643.2 |
| ETV4 | NM_001079675 |
| FAM164A | NM_016010 |
| HFE | NM_000410 |
| PKLR | NM_000298 |
| SMPD1 | NM_000543 |
| THBS1 | NM_003246 |
| UBA6 | NM_018227 |
| ZAK | NM_016653 |

There are 15 genes (Table 19) predicted overall survival of lung cancer patients in all stages, patients in stage 1, and patients in stage 1B from Shedden et al (1).

TABLE 19
15 of the 25 prognostic signature genes predict overall survival in
lung cancer patients from Shedden et al (1) with all tumor stages,
stage 1 tumors, and stage 1B tumors.

| Gene Symbol | Sequence ID |
|---|---|
| ANKHD1 | NM_017747 |
| ATP6V0D1 | NM_004691 |
| CCDC99 | NM_017785 |
| CD27 | NM_001242 |
| DKFZp434H1419 | |
| DLC1 | NM_182643.2 |
| ETV4 | NM_001079675 |
| FAM164A | NM_016010 |
| HFE | NM_000410 |
| PKLR | NM_000298 |
| SCLY | NM_016510 |
| SMPD1 | NM_000543 |
| THBS1 | NM_003246 |
| UBA6 | NM_018227 |
| ZAK | NM_016653 |

There are 16 genes (Table 20) predicted overall survival of lung cancer patients in all stages, patients in stage 1, and patients in stage 1B from Shedden et al (1).

TABLE 20
16 of the 25 prognostic signature genes predict overall survival in
lung cancer patients from Shedden et al (1) with all tumor stages,
stage 1 tumors, and stage 1B tumors.

| Gene Symbol | Sequence ID |
|---|---|
| ANKHD1 | NM_017747 |
| ATP6V0D1 | NM_004691 |
| CCDC99 | NM_017785 |
| CD27 | NM_001242 |
| DKFZp434H1419 | |
| DLC1 | NM_182643.2 |
| ETV4 | NM_001079675 |
| FAM164A | NM_016010 |
| HFE | NM_000410 |
| PDPK1 | NM_002613 |
| PKLR | NM_000298 |
| SCLY | NM_016510 |
| SMPD1 | NM_000543 |
| THBS1 | NM_003246 |
| UBA6 | NM_018227 |
| ZAK | NM_016653 |

There are 17 genes (Table 21) predicted overall survival of lung cancer patients in all stages, patients in stage 1, and patients in stage 1B from Shedden et al (1).

TABLE 21
17 of the 25 prognostic signature genes predict overall survival in
lung cancer patients from Shedden et al (1) with all tumor stages,
stage 1 tumors, and stage 1B tumors.

| Gene Symbol | Sequence ID |
|---|---|
| ANKHD1 | NM_017747 |
| ATP6V0D1 | NM_004691 |
| CCDC99 | NM_017785 |
| CD27 | NM_001242 |
| DKFZp434H1419 | |
| DLC1 | NM_182643.2 |
| ETV4 | NM_001079675 |
| FAM164A | NM_016010 |
| HFE | NM_000410 |
| PDPK1 | NM_002613 |
| PKLR | NM_000298 |
| SCLY | NM_016510 |
| SMPD1 | NM_000543 |
| STK24 | NM_001032296 |
| THBS1 | NM_003246 |
| UBA6 | NM_018227 |
| ZAK | NM_016653 |

There are 18 genes (Table 22) predicted overall survival of lung cancer patients in all stages, patients in stage 1, and patients in stage 1B from Shedden et al (1).

TABLE 22
18 of the 25 prognostic signature genes predict overall survival in
lung cancer patients from Shedden et al (1) with all tumor stages,
stage 1 tumors, and stage 1B tumors.

| Gene Symbol | Sequence ID |
|---|---|
| ANKHD1 | NM_017747 |
| ATP6V0D1 | NM_004691 |
| CCDC99 | NM_017785 |
| CD27 | NM_001242 |
| DKFZp434H1419 | |
| DLC1 | NM_182643.2 |
| ETV4 | NM_001079675 |
| FAM164A | NM_016010 |
| HFE | NM_000410 |
| PDPK1 | NM_002613 |
| PKLR | NM_000298 |
| SCLY | NM_016510 |
| SMPD1 | NM_000543 |
| STK24 | NM_001032296 |
| THBS1 | NM_003246 |
| UBA6 | NM_018227 |
| XPO1 | NM_003400 |
| ZAK | NM_016653 |

There are 19 genes (Table 23) predicted overall survival of lung cancer patients in all stages, patients in stage 1, and patients in stage 1B from Shedden et al (1).

TABLE 23
19 of the 25 prognostic signature genes predict overall survival in
lung cancer patients from Shedden et al (1) with all tumor stages,
stage 1 tumors, and stage 1B tumors.

| Gene Symbol | Sequence ID |
|---|---|
| ANKHD1 | NM_017747 |
| ATP6V0D1 | NM_004691 |
| CCDC99 | NM_017785 |
| CD27 | NM_001242 |
| DKFZp434H1419 | |
| DLC1 | NM_182643.2 |
| EMID1 | NM_133455 |
| ETV4 | NM_001079675 |
| FAM164A | NM_016010 |
| HFE | NM_000410 |
| PDPK1 | NM_002613 |
| PKLR | NM_000298 |
| SCLY | NM_016510 |
| SMPD1 | NM_000543 |
| STK24 | NM_001032296 |
| THBS1 | NM_003246 |
| UBA6 | NM_018227 |
| XPO1 | NM_003400 |
| ZAK | NM_016653 |

There are 20 genes (Table 24) predicted overall survival of lung cancer patients in all stages, patients in stage 1, and patients in stage 1B from Shedden et al (1).

TABLE 24
20 of the 25 prognostic signature genes predict overall survival in
lung cancer patients from Shedden et al (1) with all tumor stages,
stage 1 tumors, and stage 1B tumors.

| Gene Symbol | Sequence ID |
|---|---|
| ANKHD1 | NM_017747 |
| ATP6V0D1 | NM_004691 |
| CCDC99 | NM_017785 |
| CD27 | NM_001242 |
| DKFZp434H1419 | |
| DLC1 | NM_182643.2 |
| EMID1 | NM_133455 |
| ETV4 | NM_001079675 |
| FAM164A | NM_016010 |
| HFE | NM_000410 |
| PDPK1 | NM_002613 |
| PKLR | NM_000298 |
| SCLY | NM_016510 |
| SMPD1 | NM_000543 |
| STK24 | NM_001032296 |
| THBS1 | NM_003246 |
| UBA6 | NM_018227 |
| XPO1 | NM_003400 |
| ZAK | NM_016653 |
| ZNF343 | NM_024325 |

There are 22 genes (Table 25) predicted overall survival of lung cancer patients in all stages, patients in stage 1, and patients in stage 1B from Shedden et al (1).

TABLE 25
22 of the 25 prognostic signature genes predict overall survival in
lung cancer patients from Shedden et al (1) with all tumor stages,
stage 1 tumors, and stage 1B tumors.

| Gene Symbol | Sequence ID |
|---|---|
| ANKHD1 | NM_017747 |
| ATP6V0D1 | NM_004691 |
| CCDC99 | NM_017785 |
| CD27 | NM_001242 |
| DKFZp434H1419 | |
| DLC1 | NM_182643.2 |
| EMID1 | NM_133455 |
| ETV4 | NM_001079675 |
| FAM164A | NM_016010 |
| HFE | NM_000410 |
| LMF1 | NM_022773 |
| PDPK1 | NM_002613 |
| PKLR | NM_000298 |
| PTPN4 | NM_002830 |
| SCLY | NM_016510 |
| SMPD1 | NM_000543 |
| STK24 | NM_001032296 |
| THBS1 | NM_003246 |
| UBA6 | NM_018227 |
| XPO1 | NM_003400 |
| ZAK | NM_016653 |
| ZNF343 | NM_024325 |

There are 23 genes (Table 26) predicted overall survival of lung cancer patients in all stages, patients in stage 1, and patients in stage 1B from Shedden et al (1).

TABLE 26
23 of the 25 prognostic signature genes predict overall survival in
lung cancer patients from Shedden et al (1) with all tumor stages,
stage 1 tumors, and stage 1B tumors.

| Gene Symbol | Sequence ID |
|---|---|
| ANKHD1 | NM_017747 |
| ATP6V0D1 | NM_004691 |
| CCDC99 | NM_017785 |
| CD27 | NM_001242 |
| DKFZp434H1419 | |
| DLC1 | NM_182643.2 |
| EMID1 | NM_133455 |
| ETV4 | NM_001079675 |
| FAM164A | NM_016010 |
| HFE | NM_000410 |
| LMF1 | NM_022773 |
| PDPK1 | NM_002613 |
| PKLR | NM_000298 |
| PTPN4 | NM_002830 |
| SCLY | NM_016510 |
| SMPD1 | NM_000543 |
| STK24 | NM_001032296 |
| THBS1 | NM_003246 |
| TXNDC13 (TMX4) | NM_021156 |
| UBA6 | NM_018227 |
| XPO1 | NM_003400 |
| ZAK | NM_016653 |
| ZNF343 | NM_024325 |

There are 24 genes (Table 27) predicted overall survival of lung cancer patients in all stages, patients in stage 1, and patients in stage 1B from Shedden et al (1).

TABLE 27
24 of the 25 prognostic signature genes predict overall survival in
lung cancer patients from Shedden et al (1) with all tumor stages,
stage 1 tumors, and stage 1B tumors.

| Gene Symbol | Sequence ID |
|---|---|
| ANKHD1 | NM_017747 |
| ATP6V0D1 | NM_004691 |
| CCDC99 | NM_017785 |
| CD27 | NM_001242 |
| DKFZp434H1419 | |
| DLC1 | NM_182643.2 |
| EMID1 | NM_133455 |
| ETV4 | NM_001079675 |
| FAM164A | NM_016010 |
| HFE | NM_000410 |
| LMF1 | NM_022773 |
| PDPK1 | NM_002613 |
| PKLR | NM_000298 |
| PTPN4 | NM_002830 |
| SCLY | NM_016510 |
| SMPD1 | NM_000543 |
| STK24 | NM_001032296 |
| THBS1 | NM_003246 |
| TTC12 | NM_017868 |
| TXNDC13 (TMX4) | NM_021156 |
| UBA6 | NM_018227 |
| XPO1 | NM_003400 |
| ZAK | NM_016653 |
| ZNF343 | NM_024325 |

All 25 genes (Table 28) predicted overall survival of lung cancer patients in all stages, patients in stage 1, and patients in stage 1B from Shedden et al (1).

TABLE 28
25 prognostic signature genes predict overall survival in lung cancer
patients from Shedden et al (1) with all tumor stages stage 1 tumors,
and stage 1B tumors.

| Gene Symbol | Sequence ID |
|---|---|
| ANKHD1 | NM_017747 |
| ATP6V0D1 | NM_004691 |
| CCDC99 | NM_017785 |
| CD27 | NM_001242 |
| DKFZp434H1419 | |
| DLC1 | NM_182643.2 |
| EMID1 | NM_133455 |
| ETV4 | NM_001079675 |
| FAM164A | NM_016010 |
| HFE | NM_000410 |
| LEPREL2 (GPR162) | NM_014262 |
| LMF1 | NM_022773 |
| PDPK1 | NM_002613 |
| PKLR | NM_000298 |
| PTPN4 | NM_002830 |
| SCLY | NM_016510 |
| SMPD1 | NM_000543 |
| STK24 | NM_001032296 |
| THBS1 | NM_003246 |
| TTC12 | NM_017868 |
| TXNDC13 (TMX4) | NM_021156 |
| UBA6 | NM_018227 |
| XPO1 | NM_003400 |
| ZAK | NM_016653 |
| ZNF343 | NM_024325 |

It was investigated if the 12-gene signature could predict response (resistant or sensitive) to four anti-cancer drug agents for treating lung cancer. Gene expression profiles of NCI-60 cell lines quantified by Affy HG-U133A platform (normalized with GCRMA method) was used in the study. The data was available from a NCI website (http://discover.nci.nih.gov/cellminer/loadDownload.do). Machine learning algorithms from WEKA 3.6 were used to build the classifiers. First, the 12-genes were ranked using RELIEFF feature selection. Then, forward selection was used to select top genes to construct the classifier to predict drug response. Results showed that the 12-gene could be used to predict the four major drug agents used in chemotherapy (Table 29). Total RNA can be extracted from the Trizol dissolved patient tumor samples. The Trizol purified RNA can be further purified using the RNeasy columns and the manufacturer's cleanup procedure (Qiagen Inc., Valencia, Calif.). The reverse transcriptase polymerase chain reaction can used to convert the high-quality single-stranded RNA samples to double-stranded cDNA, which can then be amplified and labeled with biotin. The gene expression profiles can then be quantified with Affymetrix U133A microarray plates with standard array hybridization and scanning procedures. For chemoresponse prediction, the gene expression profiles in cell cultures can be derived from patient tumors to predict drug response. Alternatively, one could also use gene expression profiles of these 12 genes in tumor resections to predict chemoresponse. A probability of chemosensitivity of greater than 0.5 is classified as sensitive, otherwise it is classified as resistant.

TABLE 29
Prediction accuracy of chemoresponse in NCI-60 cell ines using 12-gene signature.

| | Sensitivity | Specificity | | |
|---|---|---|---|---|
| Drug | (chemoresistance) | (chemosensitivity) | Overall accuracy | P-value* |
| Carboplatin | 76% (19/25) | 80% (16/20) | 78% (35/45) | 0.003 |
| Paclitaxel (Taxol) | 87% (13/15) | 72% (8/11) | 81% (21/26) | 0.009 |
| Cisplatin | 85% (22/26) | 74% (14/19) | 80% (36/45) | 0.001 |
| Etoposide | 80% (16/20) | 67% (14/21) | 73% (30/41) | 0.016 |

*P-value < 0.05 represents the overall accuracy is significantly higher than that of random prediction (one-tailed Z-test).

Since feature selections were used to select a refined set of genes from the 12-gene prognostic signature to predict response to the drugs, different gene subsets were selected to construct the classifiers with performance listed in Table 29. In addition, different machine learning algorithms were used to construct response prediction classifiers for different drugs. A normalized Gaussian radial basis function network (RBF Network) was used to model the classifier to predict response to Carboplatin. K-nearest neighbor (k=3) algorithm was used to construct the classifier to predict response to Paclitaxel. Meta-learning algorithms DECORATE with PART as the base learner was used to construct the classifier to predict response to Cisplatin. DECORATE constructs the classifier based on ensembles of base learners and use a set of artificial training examples to create diversity in ensembles of classifiers. PART is a rule-based algorithm that uses partial decision tress to obtain rules. Adaboost M1 boosting method with Random Tree as the base learner was used to construct the classifier to predict response to Etoposide. Results were summarized in Table 30.

TABLE 30
Machine learning algorithm and genes used in predicting the chemoresponse using
12-gene signature.

| Anti-cancer | Machine learning | | Resistant lung | Sensitive lung |
|---|---|---|---|---|
| Agent | algorithm | Genes Selected | cancer cell lines | cancer cell lines |
| Carboplatin | RBF Network (seed = | ATP6V0D1 | LC: EKVX | LC: NCI_H460 |
| | 2) | CCDC99 | LC: NCI_H322M | LC: NCI_H522 |
| | | FAM164A | | (LC: NCI_H23 not |
| | | LMF1 | | included due to |
| | | PDPK1 | | missing values) |
| | | PKLR | | |
| | | SCLY | | |
| | | SMPD1 | | |
| | | STK24 | | |
| | | XPO1 | | |
| Paclitaxel | IBK (k = 3) | CCDC99 | LC: HOP_92 | LC: NCI_H460 |
| | | DLC1 | LC_EKVX | LC: NCI_H522 |
| | | LMF1 | | |
| | | PKLR | | |
| | | SMPD1 | | |
| | | XPO1 | | |
| | | ZAK | | |
| Cisplatin | Decorate (PART as | ATP6V0D1 | LC: NCI_H226 | LC: HOP_62 |
| | base learner) | CCDC99 | LC: EKVX | LC: NCI_H460 |
| | | FAM164A | LC: NCI_H322M | (LC: NCI_H23 not |
| | | LMF1 | | included due to |
| | | | | missing values) |
| Etoposide | AdaBoostM1 (seed = | CCDC99 | LC: EKVX | LC: HOP_62 |
| | 2, Random Tree as | LMF1 | LC: NCI_H322M | LC: NIC_H460 |
| | base learner) | SCLY | | |
| | | STK24 | | |
| | | XPO1 | | |

Target polynucleotide molecules can be extracted from a sample taken from an individual afflicted with non-small cell lung cancer. The sample may be collected in any clinically acceptable manner, but must be collected such that marker-derived polynucleotides (i.e., RNA) are preserved. mRNA or nucleic acids derived there from (i.e., cDNA or amplified DNA) can be labeled distinguishably from standard or control polynucleotide molecules, and both are simultaneously or independently hybridized to a detection mechanism. A detection mechanism can be any standard comparison mechanism such as a microarray or an assay of reverse transcription polymerase chain reaction (RT-PCR) comprising some or all of the markers or marker sets or subsets described above. This process identifies positive matches. Alternatively, mRNA or nucleic acids derived therefrom may be labeled with the same label as the standard or control polynucleotide molecules to identify positive matches, wherein the intensity of hybridization of each at a particular probe or primer is compared for such an identification. A sample may include any clinically relevant tissue sample, such as a tumor biopsy or fine needle aspiration, or a sample of bodily fluid, such as blood, plasma, serum, lymph, ascetic fluid, cystic fluid, or urine. The sample may be taken from a human, or from non-human animals such as horses, mice, ruminants, swine or sheep. Patients' gene expression levels may be quantified by any means known in the art based on the marker sets defined above. Patients may be classified based on the quantitative expression profiles using any means of classification known in the art. A means of classification can be, for example, the risk scores of a patient cohort may be generated using a Cox proportional hazard model. Patients with a risk score greater than the median is defined as high risk, whereas patients with a risk score less than the median is classified as low risk. Alternatively, a patient may be classified as high risk if this patient's gene expression profile is correlated with the high risk signature, or classified as low risk if this patient's gene expression profile is correlated with the low risk signature. A patient's prognostic categorization can also be determined by using a statistical model or a machine learning algorithm, which computes the probability of recurrence based on this patient's gene expression profiles. Cutoffs can be defined for patient stratification based on specific clinical setting. In addition, patients may be defined into three risk groups in the prognostic categorization based on the marker sets defined above.

Methods for preparing total and poly(A)+RNA are well known and are described in (11). RNA may be isolated from eukaryotic cells by procedures that involve cell lysis and denaturation of the proteins contained therein. Cells of interest include wide-type cells (i.e., no mutation), drug-treated wild-type cells, tumor- or tumor-derived cells, modified cells, normal or tumor cell lines cells, and drug-treated modified cells. Total RNA may also be extracted from samples using commercially available kits such as the RNeasy mini kit according the manufacturer's protocol (Qiagen, USA).

Additional steps may be performed to remove DNA (11). If desired, RNase inhibitors may be added to the lysis buffer. Likewise, a protein denaturation/digestion step may be added to the protocol. mRNA may be purified by means such as magnetic separation using Dynabeads (Dynal) or the Invitrogen FastTrack 2.0 kit (12).

For many applications, it is desirable to preferentially enrich mRNA with respect to other cellular RNAs, such as transfer RNA (tRNA) and ribosomal RNA (rRNA). Total RNA may also be linearly amplified using the original or modified Eberwine method (13) and be used as a reference for cDNA analysis (14).

The sample of RNA can comprise a plurality of different mRNA molecules, each different mRNA molecular having a different nucleotide sequence. In a specific embodiment, the RNA sample has not been functionally annotated.

A set of biomarkers for the identification of conditions of indications associated with lung cancer may be used. Generally, the markers sets were identified by determining which of ˜22,000 human genes had expression patterns that correlated with the conditions or indications.

In one embodiment, the expression of all markers in a sample can be compared to the expression of all markers in the gene signatures as described above. The comparison may be accomplished by any means known in the art. For example, the expression level may be determined by isolating and determining the level (i.e., the abundance) of nucleic acid transcribed from each marker gene. Alternatively, or additionally, the level of specific proteins translated from mRNA transcribed from a marker gene may be determined. For example, expression levels of various markers may be measured by separation of target nucleotide molecules (e.g., RNA or cDNA) derived from the markers in agarose or polyacrylamide gels, followed by hybridization with, marker-specific oligonucleotide probes. Alternatively, the comparison may be accomplished by the labeling of target polynucleotide molecules followed by separation on a sequence gel. The comparison may also be accomplished by measuring the gene expression level using real-time reverse transcription polymerase chain reaction with marker-specific primers/probes. Patients may be classified based on the quantitative expression profiles using any means known in the art. For example, the risk scores of a patient cohort may be generated using a Cox proportional hazard model. Patients with a risk score greater than the median is defined as high risk, whereas patients with a risk score less than the median is classified as low risk. Alternatively, a patient may be classified as high risk if this patient's gene expression profile is correlated with the high risk signature, or classified as low risk if this patient's gene expression profile is correlated with the low risk signature. A patient's prognostic categorization can also be determined by using a statistical model or a machine learning algorithm, which computes the probability of recurrence based on this patient's gene expression profiles. Cutoffs can be defined for patient stratification based on specific clinical setting. In addition, patients may be defined into three risk groups in the prognostic categorization based on the marker sets defined above. Similarly, tumor stage and tumor differentiation can be determined with the marker subsets as described above with any means known in the art.

A 12-gene survival marker was selected based on its predictive power of postoperative survival outcome. A combination of t-test, significance analysis of microarrays (SAM), and RELIEFF feature selection was used to identify this gene signature. Different-variance t-test was first used to identify 718 genes from 22,283 genes; As an alternative, SAM method implemented in software MultiExperiment Viewer (MeV) identified a set of 1,431 genes. 583 genes common in these two sets of genes were identified and this common gene list was further refined using RELEFF with software WEKA. By applying forward selection from the top of the list based on the ranking from RELIEFF, 12 genes (Table 1) were selected as the set of signature gene for predicting lung cancer postoperative survival outcome.

A 15-gene survival marker was selected based on its predictive power of postoperative survival outcome. A combination oft-test and RELIEFF feature selection was used to identify this gene signature. First, equal-variance t-test was used to identify 689 genes from 22,283 genes. Then, RELEFF was used to further refine the gene signature with software WEKA. By applying forward selection from the top of the list based on the ranking from RELIEFF, 15 genes (Table 1) were selected as the set of signature gene for predicting lung cancer postoperative survival outcome.

A 16-gene survival marker was selected based on its predictive power of postoperative survival outcome. A combination oft-test, significance analysis of microarrays (SAM), RELIEFF feature selection, and biological function study was used to identify this gene signature. First, a combination oft-test, SAM, and RELIEFF was used to identify a set of 12-gene and a set of 15-gene signature (section [0026], [0027]). Then, biological function study was done on these two gene sets using software Ingenuity Pathway Analysis (IPA). The 16 genes sharing common biological functions revealed from the study were selected as the set of signature gene for predicting lung cancer postoperative survival outcome.

Marker selection algorithms include statistics methods and machine learning algorithms. Statistics methods, t-test in software package R (found at found at http://www.r-project.org) and significance analysis of microarray (SAM) of software MultiExperiment Viewer (MeV, found at www.tm4.org/mev/) are used. Feature selection algorithm, RELIEFF used is implemented in software package WEKA 3.4, (found at http://www.cs.waikato.ac.nz/ml/weka/).

Significance analysis of microarrays (SAM) measures the differentiation of genes based on the ratio change in gene expression relative to standard deviation in the data for each gene. The standard deviation is measure based on repeated expression measurements. Furthermore, SAM computes false discovery rate (FDR) based on permutation to adjust for multiple hypothesis testing problems in selecting significant genes among huge number of genes (15).

RELIEFF is an algorithm proposed by Kononenko et al. (16) that ranks attributes based on their differences between two classes. It is an extension to the RELIEF algorithm proposed by Kira and Rendell (17). In the RELLIEF algorithm, each sample is randomly selected and weight of features is computed based on the values of features of its nearest sample of the same class (hit) and values of features of its nearest sample of different class (miss). Specifically, function cliff (Attribute, InstanceA, InstanceB) calculates the difference between the values of Attribute for two instances. The difference between the selected sample and its nearest miss would be added to the current weight; where the different between the selected sample and its nearest hit would be subtracted from the current weight. Thus, when the algorithm stops after repeating the process a specific number of times, features that differentiated between samples of different classes will have higher weights awarded. Instead of the nearest miss and nearest hits, k-nearest hits and k-nearest misses of the randomly selected sample are used in RELIEFF. In addition, a more reliable probabilities estimation method is implemented in RELIEFF.

Prediction methods used in the study includes a supervised machine learning algorithms in software package WEKA 3.4 and a statistics model in software package R. Specifically, Naïve Bayes was used to construct survival prediction models with the 12-gene signature; Cox proportional hazard model was used to develop models to predict survival outcome with the 15 genes or the 16 genes as covariates.

Naïve Bayes classifier is a machine learning method based on Bayes theorem and with the assumption that attributes are conditionally independent given the target class. A new sample with attribute values <a₁, a₂, . . . , aᵢ> would be classified into the most probable class based on posterior probability from the Bayes theorem (18). In other words, the new sample would be classified into the class with the highest posterior probability, based on the following expression:

C_{predicted} =argmax_{cj∈C}P(a₁, a₂, . . . , aᵢ|cⱼ)P(cⱼ)

where C is the set containing all the classes for the problem and cⱼ is a specific class. Based on the conditional independence assumption, it holds true for the situation that given a class of the instance, the probability of observing the conjunction of attributes a₁, a₂, . . . , aᵢ would be the product of the probability of the individual attributes:

P(a₁, a₂, . . . , aᵢ|cⱼ)=ΠᵢP(aᵢ|cⱼ)

Therefore, a simpler form of equation (1) to be deployed in Naïve Bayes classifier is expressed as:

$${c}_{predicted} = {argmax}_{{c}_{j} ∈ C} P ( {c}_{j} ) {\prod }_{i} P ( {a}_{i} \vert {c}_{j} )$$

Cox proportional hazard model, or usually know as Cox model, is a common statistical technique used in survival analysis to study the relationships between independent variables (or covariates) and the survival outcome of patients. It estimates the degree of effect of independent variables on survival outcome. It's a semi-parametric regression model because it integrates two parts: a non-parametric hazard function and a parametric multi-regression model.

The hazard function is non-parametric because it makes no assumption on distribution of the survival time. The hazard function, denoted by h(t), gives the probability that a patient will experience an event (such as death) within a small time interval, given that the individual has survived up to the beginning of the interval (which is at time t). It's the risk of the event from happening (such as dying) at time t (19). This can be expressed by the following formula:

$$h ( t ) = \frac{\begin{matrix}number of patients experiencing \\ an event in interval beginning at t\end{matrix}}{( number of patients surviving at time t ) \times ( interval width )}$$

The parametric multi-regression part implemented in Cox model is used to estimate the effects of multiple independent variables on the hazard of the event. It is similar to multiple regression technique, but it allows multiple independent variables to be taken into account at once at any time t. Therefore, the hazard of an event at time t could be expressed by formula:

h(t)=h₀(t)xexp(β₁·x₁+β₂·x₂+ . . . +βₙ−xₙ)

Or the natural logarithmic form:

ln h(t)=ln h₀(t)+β₁·x₁+β₂·x₂+ . . . +βₙ·xₙ

where x₁ to xₙ are n independent variables, and β₁ to βₙ are regression coefficients of each independent variable. In Cox model, these regression coefficients are estimated using maximum likelihood estimation.
h₀(t) is known as baseline hazard function. It is the probability that patients will experience the event when all other independent variables are zero.
From these two equations, h(t) and ln h(t), we could notice that each regression coefficients represents the proportional change that can be expected in the hazard. In addition, these effects of independent variables act additively on the hazard and remain constant over time. Since there's a constant relationship between independent variables and the survival outcome, Cox model is considered a proportional hazard model.

To use Cox proportional hazard model to construct a prognostic classifier, a model is first constructed by fitting signature genes as covariates into the Cox model on training data. Then, regression coefficients estimated from the fitted model are used to compute risk score for all patients. By defining a cutoff value based on risk scores, classification could be made. For example, a cutoff value is defined to be the median value of risk scores from patients samples in training data; the classification scheme would be classifying samples with risk score less than the cutoff value as low-risk patients and samples with risk score greater than or equal to the cutoff value as high-risk patients.

Validation methods used include statistical metrics and bioinformatics methods. Statistical metric concordance probability estimate (CPE) in software R and multivariate analysis were used to evaluate the prediction performance with respect to true survival outcome of patients. Bioinformatics tools Gene Set Enrichment Analysis (GSEA) (found at http://www.broadinstitute.org/gsea/) was used to assess the association of the gene signature to the survival status

In general, concordance probability is used to evaluate how the predicted outcomes of a nonlinear statistical model agreed with the actual outcomes. The estimation of concordance probability proposed by Gonen and Heller (20), which is an estimation of concordance probability within the framework of the Cox model can be used. Since the concordance probability estimation proposed focused on Cox model, the concordance probability is thus defined as:

K(β)=P(T₂>T₁|β_{T}x₁≧β^{T}x₂)

where T is the response variable (the actual survival outcomes of patient samples) and βₓ^{T} corresponds to risk scores obtained from the Cox model. In the estimation, partial likelihood estimator \hat{β} is used to substitute β and the empirical distribution of βₓ^{T} is used to represent the distribution of risk scores. To resolve the asymptotic nature of the Cox partial likelihood estimator, a kernel function is used for smoothing. The final estimator used in obtaining the concordance probability of the model obtained would be purely based on the regression coefficients and covariates from Cox model, without patients' survival time and outcomes. Therefore, this estimation is not sensitive to the censoring cases in the patient samples. If the concordance probability estimate (CPE) obtained is close to 0.5, it indicates that model has poor predictive on the actual survival outcome (it's as good as the random chance). The model showed better predictive performance when the CPE is approaching closer to 1.

GSEA allows assessment of gene sets in the genome-wide expression profiles (21). Based on the genome-wide gene expression profiles of a set of patients and their respective phenotype (i.e. survival outcome), GSEA would determine how the members in the gene set correlated to the phenotypes. In GSEA, according to the differential expression between the classes found in the provided input, it maintained a ranked list of genes (L). Then, a measurement called enrichment score (ES) would be computed for each gene set using running-sum statistics with weighted correlation of the genes with the phenotype. ES reflects the degree to which a gene set is overrepresented to both ends of L. A statistical significance (nominal P value) would also be estimated using phenotype-based permutation test. If a gene set is significantly overrepresented with respect to the phenotypes (either one or both), then it would have extreme ES at both ends of the ranked list L. GSEA also allows comparisons of multiple gene sets. In assessment of multiple gene sets, permutation test is implemented in the algorithm to account for multiple hypothesis testing. Thus, the ES would be normalized by the mean of scores from permutations, resulting normalized enrichment score (NES). Similarly, instead of nominal P value, false discovery rate (FDR) corresponding to the NES of each gene set is calculated based on permutations. FDR estimates the probability that the gene set with the given NES represents a false positive finding.

Functional Pathway Analysis. Interactions among signature genes with recognized lung cancer hallmark genes in functional pathways are studied using Ingenuity Pathway Analysis (IPA) software (found at http://www.ingenuity.com/) and Pathway Studio 7 (found at http://www.ariadnegenomics.com/products/pathway-studio/).

IPA enables analysis of biological functions of a set of genes based on its proprietary comprehensive knowledge database, which was curated by experts. These functions include functions related to diseases, molecular functions, or cellular processes. In addition, it revealed the significant pathways in which the set of genes involved. In addition, it revealed the significant pathways in which the set of genes involved.

Pathway Studio is pathway analysis software with a proprietary database ResNet with curated interactions. It allows users to explore interactions among a set of genes based on the database. ResNet database gathers data from publications available through PubMed using Ariadne's MedScan tecnnology. In addition, Pathway Studio allows users to extend their own databases by importing additional publications.

The prediction of patient outcome may be accomplished with any means known in the art. For example, to estimate a patient's recurrent and metastatic potential, risk scores are generated by fitting the identified gene predictors in a Cox proportional hazard model as covariates. A higher risk score represents a higher probability of tumor recurrence. The distribution of the risk scores can be used to classify the patients into three groups: high-risk, low-risk, and intermediate-risk. Alternatively, patients may be stratified into two groups: high- or low-risk. Kaplan-Meier analysis may be used to assess the disease-free survival probability of three risk groups in the studied patient cohorts. Similarly, a Cox proportional hazard model may be developed to estimate a patient's overall survival probability. A higher survival risk score represents a higher risk for death from lung cancer. Alternatively, machine learning algorithms such as Random Committee, Bayesian belief networks, and artificial neural networks may be used to determine group membership for diagnostic and prognostic categorization, including tumor stage, differentiation, and risk for recurrence.

For prognostic predictions in clinic, the expression levels of the markers can be measured with any means known in the art such as cDNA microarrays (12;14;22), various generations of Affymetrix gene chips (Affymetrix, Santa Clara, Calif.), and real-time reverse transcription polymerase chain reactions. Kits comprising the marker sets above can be utilized. The analytical methods described above can be implemented by use of following computer systems. For example, a computer system can be an Intel 8086-, 80386-, 80486-, or Pentium-based process with preferably 64 MB or more of main memory. The computer system can be linked to an external component, including mass storage. This mass storage can be one or more hard disks, preferably of 1GB or more storage capacity. Other external components include regular accessories for a computer such as a monitor, a mouse, or a printer.

The software program described in above sections can be implemented with software packages R and WEKA. The software to be included in the kit comprises the data analysis methods as disclosed herein. In particular, the software algorithms may include mathematical procedures for biomarker discovery, including the computation of the conditional probability with clinical categories (i.e., relapse status) and marker expression. The software may also include mathematical procedures for computing the regression coefficients between the marker expression and patient survival.

Alternative computer systems and software for implementing the analytical methods will be apparent to one of skill in the art and are intended to be comprehended within the accompanying claims.

These terms and specifications, including the examples, serve to describe the invention by example and not to limit the invention. It is expected that others will perceive differences, which, while differing from the forgoing, do not depart from the scope of the invention herein described and claimed. In particular, any of the function elements described herein may be replaced by any other known element having an equivalent function.

### REFERENCE LIST

1. Shedden K, Taylor J M, Enkemann S A et al. Gene expression-based survival prediction in lung adenocarcinoma: a multi-site, blinded validation study. Nat Med 2008;14:822-7.
2. Lu Y, Lemon W, Liu P Y et al. A gene expression signature predicts survival of patients with stage I non-small cell lung cancer. PLoS Med 2006;3:e467.
3. Beer D G, Kardia S L, Huang C C et al. Gene-expression profiles predict survival of patients with lung adenocarcinoma. Nat Med 2002;8:816-24.
4. Bhattacharjee A, Richards W G, Staunton J et al. Classification of human lung carcinomas by mRNA expression profiling reveals distinct adenocarcinoma subclasses. Proc Natl Acad Sci USA 2001;98:13790-5.
5. Chen H Y, Yu S L, Chen C H et al. A five-gene signature and clinical outcome in non-small-cell lung cancer. N Engl J Med 2007;356:11-20.
6. Boutros P C, Lau S K, Pintilie M et al. Prognostic gene signatures for non-small-cell lung cancer. Proc Natl Acad Sci USA 2009;106:2824-8.
7. Guo L, Ma Y, Ward R et al. Constructing molecular classifiers for the accurate prognosis of lung adenocarcinoma. Clin Cancer Res 2006;12:3344-54.
8. Lau S K, Boutros P C, Pintilie M et al. Three-gene prognostic classifier for early-stage non small-cell lung cancer. J Clin Oncol 2007;25:5562-9.
9. Potti A, Mukherjee S, Petersen R et al. A genomic strategy to refine prognosis in early-stage non-small-cell lung cancer. N Engl J Med 2006;355:570-80.
10. Raponi M, Zhang Y, Yu J et al. Gene expression signatures for predicting prognosis of squamous cell and adenocarcinomas of the lung. Cancer Res 2006;66:7466-72.
11. Sambrook J, Russell D W. Molecular Cloning: A Laboratory Manual. Cold Spring Harbor Laboratory Press, 2001.
12. Sorlie T, Perou C M, Tibshirani R et al. Gene expression patterns of breast carcinomas distinguish tumor subclasses with clinical implications. Proc Natl Acad Sci USA 2001;98:10869-74.
13. Eberwine J, Yeh H, Miyashiro K et al. Analysis of Gene Expression in Single Live Neurons. PNAS 1992;89:3010-4.
14. Sotiriou C, Neo S Y, McShane L M et al. Breast cancer classification and prognosis based on gene expression profiles from a population-based study. Proc Natl Acad Sci USA 2003;100:10393-8.
15. Tusher V G, Tibshirani R, Chu G. Significance analysis of microarrays applied to the ionizing radiation response. Proc Nall Acad Sci USA 2001;98:5116-21.
16. Kononenko I, Simec E, Robnik-Sikonja M. Overcoming the Myopia of Inductive Learning Algorithms with RELIEFF. Applied Intelligence 1997;7:39-55.
17. Kira K, Rendell L. A Practical Approach to Feature Selection. Proceedings of the Ninth International Workshop on Machine Learning (Aberdeen, Scotland, UK) 1992;249-56.
18. Mitchell T M. Machine Learning. McGraw-Hill International Editions. Bayesian Learning. 1997:154-99.
19. Stephen J. Walters. What is a Cox model. What is ? series 2007;1.
20. Gonen M, Heller G. Concordance probability and discriminatory power in proportional hazards regression. Biometrika 2005;92:965-70.
21. Subramanian A, Tamayo P, Mootha V K et al. Gene set enrichment analysis: A knowledge-based approach for interpreting genome-wide expression profiles. Proceedings of the National Academy of Sciences of the United States of America 2005;102:15545-50.
22. van 't Veer L J, Dai H, van de Vijver M J et al. Gene expression profiling predicts clinical outcome of breast cancer. Nature 2002;415:530-6.

What is claimed is:

1. A method comprising creating a sample by extracting target polynucleotide molecules from an individual afflected with non-small cell lung cancer so that the RNA is preserved, deriving the mRNA from the mRNA of the individual, labeling the mRNA and hybridizing to a detection mechanism containing 12 or more of Seq ID No. 1, Seq. ID No. 2, Seq ID No. 3, Seq ID No. 4, Seq ID No. 5, Seq ID No. 6, Seq ID No. 7, Seq ID No. 8, Seq ID No. 9, Seq ID No. 10, Seq ID No. 11, Seq ID No. 12, Seq ID No. 13, Seq ID No. 14, Seq ID No. 15, Seq ID No. 16, Seq ID No. 17, Seq ID No. 18, Seq ID No. 19, Seq ID No. 20, Seq ID No. 21, Seq ID No. 22, Seq ID No. 23, Seq ID No. 24, Seq ID No. 25 wherein the individual is classified based upon a quantitative expression profile compared to a control.

2. The method of claim 1 wherein the control is distinguishably labeled from the sample.

3. The method of claim 1 wherein the control is labeled the same as the sample.

4. The method of claim 1 wherein the detection mechanism is comprised of Seq ID No. 1, Seq. ID No. 2, Seq ID No. 3, Seq ID No. 4, Seq ID No. 5, Seq ID No. 6, Seq ID No. 7, Seq. ID No. 8, Seq ID No. 9, Seq ID No. 10, Seq ID No. 11, Seq ID No. 12, Seq ID No. 13, Seq ID No. 14, and Seq ID No. 15.

5. The method of claim 1 wherein the detection mechanism is comprised of Seq ID No. 4, Seq ID No. 7, Seq ID No. 16, Seq ID No. 17, Seq ID No. 18, Seq ID No. 19, Seq ID No. 20, Seq ID No. 21, Seq ID No. 22, Seq ID No. 23, Seq ID No. 24, and Seq ID No. 25.

6. The method of claim 1 wherein the detection mechanism is comprised of Seq ID No. 16, Seq ID No. 2, Seq ID No. 4, Seq ID No. 6, Seq ID No. 8, Seq ID No. 18, Seq ID No. 19, Seq ID No. 20, Seq ID No. 10, Seq ID No. 21, Seq ID No. 22, Seq ID No. 23, Seq ID No. 11, Seq ID No. 13, Seq ID No. 24 and Seq ID No. 25.

7. The method of claim 1 wherein the detection mechanism is comprised of Seq ID No. 1, Seq. ID No. 2, Seq ID No. 3, Seq ID No. 4, Seq ID No. 5, Seq ID No. 6, Seq ID No. 7, Seq ID No. 8, Seq ID No. 9, Seq ID No. 10, Seq ID No. 11, Seq ID No. 12, Seq ID No. 13, Seq ID No. 14, Seq ID No. 15, Seq ID No. 16, Seq ID No. 17, Seq ID No. 18, Seq ID No. 19, Seq ID No. 20, Seq ID No. 21, Seq ID No. 22, Seq ID No. 23, Seq ID No. 24, Seq ID No. 25.

8. The method of claim 5 further comprising the step of predicting a chemoresponse to cisplatin, Carboplatin, Etoposide, and paclitxel based on gene expression profiles between the drug and the detection mechanism wherein a score of greater than 0.5 on one or more of the algorithms RBF Network, IBK, Decorate, and AdaBoostMl predicts chemosensitivity.

9. The method of claim 5 further comprising the step of predicting a chemoresponse to cisplatin, Carboplatin, Etoposide, and paclitxel based on gene expression profiles of tumor resections between the drug and the detection mechanism wherein a score of greater than 0.5 on one or more of the algorithms RBF Network, IBK, Decorate, and AdaBoostMl predicts chemosensitivity.

10. A method comprising creating a sample by extracting target polynucleotide molecules from an individual afflected with non-small cell lung cancer so that the RNA is preserved, deriving the nucleic acids from the mRNA of the individual, labeling the nucleic acids and hybridizing to a detection mechanism containing 12 or more of Seq ID No. 1, Seq. ID No. 2, Seq ID No. 3, Seq ID No. 4, Seq ID No. 5, Seq ID No. 6, Seq ID No. 7, Seq ID No. 8, Seq ID No. 9, Seq ID No. 10, Seq ID No. 11, Seq ID No. 12, Seq ID No. 13, Seq ID No. 14, Seq ID No. 15, Seq ID No. 16, Seq ID No. 17, Seq ID No. 18, Seq ID No. 19, Seq ID No. 20, Seq ID No. 21, Seq ID No. 22, Seq ID No. 23, Seq ID No. 24, Seq ID No. 25 wherein the individual is classified based upon a quantitative expression profile compared to a control.

11. The method of claim 10 wherein the control is distinguishably labeled from the sample.

12. The method of claim 10 wherein the control is labeled the same as the sample.

13. The method of claim 10 wherein the detection mechanism is comprised of Seq ID No. 1, Seq. ID No. 2, Seq ID No. 3, Seq ID No. 4, Seq ID No. 5, Seq ID No. 6, Seq ID No. 7, Seq ID No. 8, Seq ID No. 9, Seq ID No. 10, Seq ID No. 11, Seq ID No. 12, Seq ID No. 13, Seq ID No. 14, and Seq ID No. 15.

14. The method of claim 10 wherein the detection mechanism is comprised of Seq ID No. 4, Seq ID No. 7, Seq ID No. 16, Seq ID No. 17, Seq ID No. 18, Seq ID No. 19, Seq ID No. 20, Seq ID No. 21, Seq ID No. 22, Seq ID No. 23, Seq ID No. 24, and Seq ID No. 25.

15. The method of claim 10 wherein the detection mechanism is comprised of Seq ID No. 16, Seq ID No. 2, Seq ID No. 4, Seq ID No. 6, Seq ID No. 8, Seq ID No. 18, Seq ID No. 19, Seq ID No. 20, Seq ID No. 10, Seq ID No. 21, Seq ID No. 22, Seq ID No. 23, Seq ID No. 11, Seq ID No. 13, Seq ID No. 24 and Seq ID No. 25.

16. The method of claim 10 wherein the detection mechanism is comprised of Seq ID No. 1, Seq. ID No. 2, Seq ID No. 3, Seq ID No. 4, Seq ID No. 5, Seq ID No. 6, Seq ID No. 7, Seq ID No. 8, Seq ID No. 9, Seq ID No. 10, Seq ID No. 11, Seq ID No. 12, Seq ID No. 13, Seq ID No. 14, Seq ID No. 15, Seq ID No. 16, Seq ID No. 17, Seq ID No. 18, Seq ID No. 19, Seq ID No. 20, Seq ID No. 21, Seq ID No. 22, Seq ID No. 23, Seq ID No. 24, Seq ID No. 25.

17. The method of claim 14 further comprising the step of predicting a chemoresponse to cisplatin, Carboplatin, Etoposide, and paclitxel based on gene expression profiles between the drug and the detection mechanism wherein a score of greater than 0.5 on one or more of the algorithms RBF Network, IBK, Decorate, and AdaBoostMl predicts chemosensitivity.

18. The method of claim 14 further comprising the step of predicting a chemoresponse to cisplatin, Carboplatin, Etoposide, and paclitxel based on gene expression profiles of tumor resections between the drug and the detection mechanism wherein a score of greater than 0.5 on one or more of the algorithms RBF Network, IBK, Decorate, and AdaBoostMl predicts chemosensitivity.
